(19) United States
(12) Patent Application Publication
(10) Pub. No.: US 2026/0014174 A1
(43) Pub. Date: Jan. 15, 2026

(54) DUAL INHIBITORS FOR HDAC3 AND HIV-1 PR

(71) Applicant: Purdue Research Foundation, West Lafayette, IN (US)

(72) Inventors: Mark A. Lipton, Lafayette, IN (US); Jean Anne Chmielewski, Lafayette, IN (US)

(73) Assignee: Purdue Research Foundation, West Lafayette, IN (US)

(21) Appl. No.: 19/327,833

(22) Filed: Sep. 12, 2025

Related U.S. Application Data
(63) Continuation-in-part of application No. PCT/US2025/036749, filed on Jul. 8, 2025.
(60) Provisional application No. 63/669,808, filed on Jul. 11, 2024.

(51) Int. Cl. A61K 31/63; A61K 31/341; A61K 31/635; C07C 317/34; C07D 307/77

(52) U.S. Cl. CPC A61K 31/63; A61K 31/341; A61K 31/635; C07C 317/34; C07D 307/77; C07B 2200/05

(57) ABSTRACT

Compounds that are dual inhibitors of histone deacetylase 3 (HDAC3) and human immunodeficiency virus type-1 (HIV-1) protease (PR); compositions comprising same; and methods of use.

### CROSS-REFERENCE TO RELATED APPLICATIONS

This application is a continuation-in-part application of international patent application no. PCT/US2025/36749, which was filed Jul. 8, 2025, and which claims priority to U.S. provisional patent application No. 63/669,808, which was filed Jul. 11, 2024, both of which are hereby incorporated by reference in their entireties.

### TECHNICAL FIELD

The present disclosure relates to compounds that are dual inhibitors of histone deacetylase 3 (HDAC3) and human immunodeficiency virus type-1 (HIV-1) protease (PR) enzymes, compositions comprising the same, and their use for inhibiting, treating, or eradicating HIV-1 infection.

### BACKGROUND

This section introduces aspects that may help facilitate a better understanding of the disclosure. Accordingly, these statements are to be read in this light and are not to be construed as admissions about what is or is not prior art.

The eradication of human immunodeficiency virus type 1 (HIV-1) from infected individuals remains a major medical challenge. Combined antiretroviral therapy (cART) leads to a decline in HIV-1 to imperceptible levels and has reduced HIV-1-related morbidity and mortality. However, discontinuing this treatment causes rapid viral rebound, requiring those infected with HIV-1 to continue cART throughout their lives to keep HIV-1 replication in check. Most people living with HIV-1 on cART still have residual and persistent viremia that mainly arises from reservoirs of replication-competent proviruses in long-lived latently infected cells. These latent reservoirs, which represent a major obstacle for HIV-1 eradication, are found in a number of cell types and anatomical sites, including resting memory CD4⁺ T-cells, macrophages, and the central nervous system.

Efforts to purge latently infected cells have focused on reactivation of the proviruses, known as a “shock and kill” approach. Upon activation of latent HIV-1, the infected cells could then be killed by host immune responses and/or virus-induced cytopathic effects (CPEs). This approach has used latency-reversing agents (LRAs), such as histone deacetylase (HDAC) inhibitors, to force the activation of proviruses in latently infected cells. The “shock and kill” approach, however, must also be implemented in concert with cART, such as HIV-1 protease (PR) inhibitors, to prevent new rounds of infections by virions released from the LRA-stimulated cells. Since the usual cART regimen combines three or more different drugs, including nucleoside and non-nucleoside reverse transcriptase inhibitors (NRTIs and NNRTIs) and PR inhibitors, the addition of LRAs (HDAC inhibitors) to cART may cause unwanted drug-drug interactions. In addition, the reservoirs of HIV-1 occur in sites where cART access is often limited, and this points to the essential need for effective co-entry of LRAs and cART into these sites as well.

There are currently no dual HIV-1 PR and HDAC3 inhibitors that have been reported. The approach currently used in the field is to treat with an HDAC3 inhibitor to stimulate the production of HIV-1 from latent cells. However, this strategy must also be implemented in concert with a separate treatment of antiretroviral (cART) agents, such as HIV-1 PR inhibitors, to prevent new rounds of infections by virions released from the stimulated cells. If HDAC inhibitors and cART drugs are unable to reach the same anatomical sites or cells, new rounds of infections by virions released from provirus-activated cells, unfortunately, occur.

Therefore, there is a need to develop a compound that is a dual inhibitor of HDAC3 and HIV-1 PR for inhibiting, treating, or eradicating HIV-1 infection by simultaneously activating HIV-1 latency within cells and preventing new infection. It is an object of the present disclosure to provide such compounds. This and other objects and advantages, as well as inventive features, will be apparent from the detailed description.

### SUMMARY

Provided is a compound of formula (T):

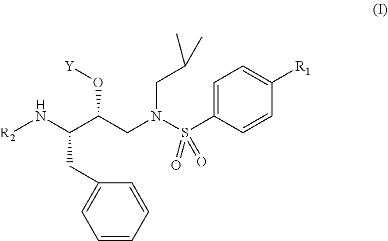

wherein Y is H, deuterium, or PO₃H;
R₁ is NH₂ or a group selected from:

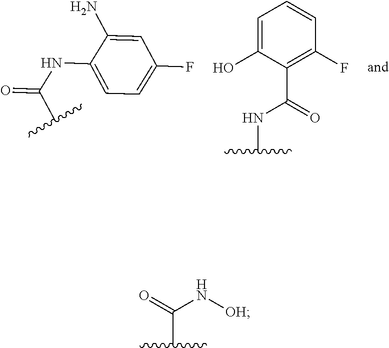

and
R₂ is

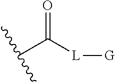

wherein L is O, C₁-C₅ alkyl, C₆-C₁₂ aryl, or C₅-C₁₂ heteroaryl; and
G is selected from the group consisting of (i), (ii), (iii), (iv), and (v):

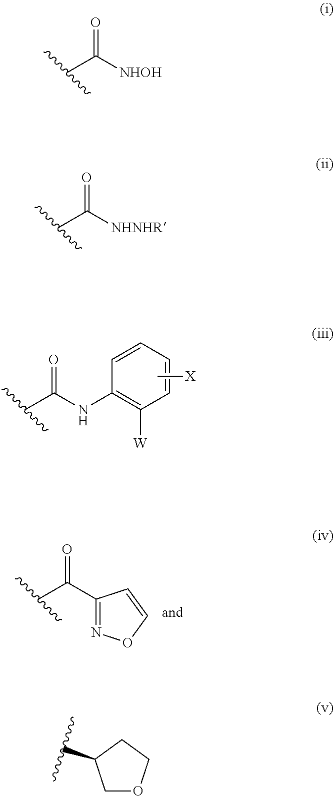

wherein R′ is C₁-C₅ alkyl;
X is H or halogen; and
W is OH, NH₂, or SCH₃;
with the proviso that when G is (v), R₁ is not NH₂,
or a pharmaceutically acceptable salt, hydrate, tautomer, or optical isomer thereof.

In some embodiments of the compound of formula (I), R₁ is the group selected from:

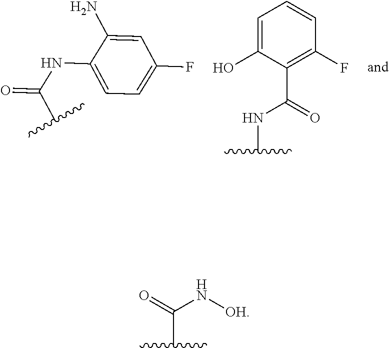

In some embodiments of the compound of formula (I), R₁ is NH₂; and
R₂ is

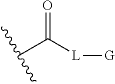

wherein L is O, C₁-C₅ alkyl, C₆-C₁₂ aryl, or C₅-C₁₂ heteroaryl; and
G is selected from the group consisting of (i), (ii), (iii), and (iv):

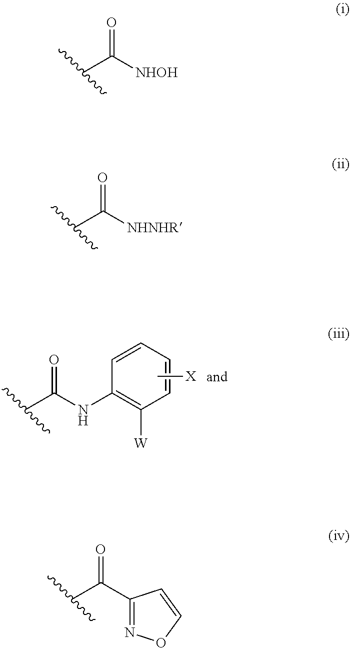

wherein R′ is C₁-C₅ alkyl;
X is H or halogen; and
W is OH, NH₂, or SCH₃.

In some embodiments of the compound of formula (I), R₂ is

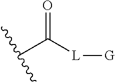

wherein L is O; and
G is

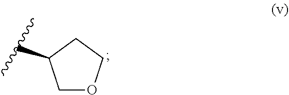 [SMILES: *[C@H]1CCOC1]

and
R₁ is selected from:

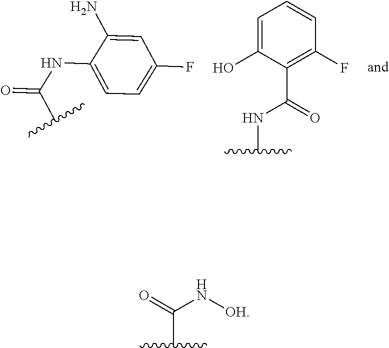

with the proviso that when G is (v), R₁ is not NH₂.

In some embodiments of the compound of formula (I), R₂ is

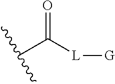

wherein L is C₆-C₁₂ aryl or C₅-C₁₂ heteroaryl; and G is selected from the group consisting of (i), (ii), and (iii):

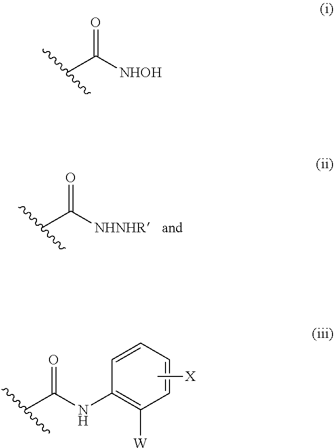

wherein R′ is C₁-C₅ alkyl;
X is H or halogen; and
W is NH₂.
   In some embodiments of the compound of formula (I), L is phenyl or pyridyl.

In some embodiments, the compound of formula (I) is a compound of formula (IA):

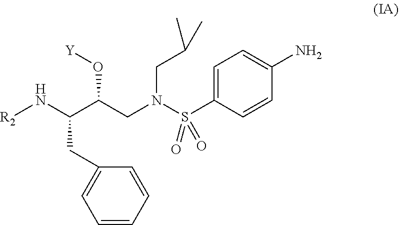

wherein Y is H, deuterium, or PO₃H; and
R is selected from the group consisting of (a), (b), (c) and (d):

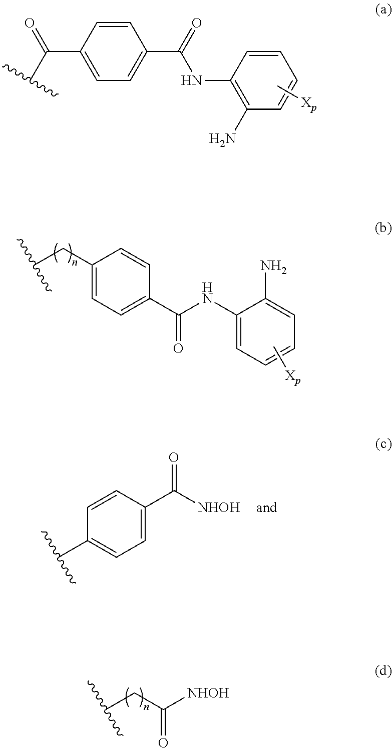

wherein Xₚ is halogen, wherein p is an integer from 1 to 4; and
n is an integer from 0 to 6;
or a pharmaceutically acceptable salt, hydrate, tautomer, or optical isomer thereof.

In some embodiments of the compound of formula (IA), X is fluorine.

In some embodiments of the compound of formula (IA), R is:

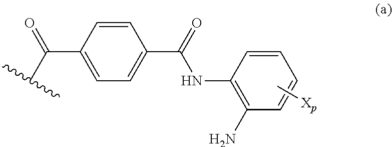

wherein Xₚ is as defined above.

In some embodiments of the compound of formula (IA), R is:

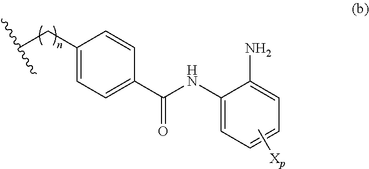

wherein Xₚ and n are as defined above.

In some embodiments of the compound of formula (IA), R is:

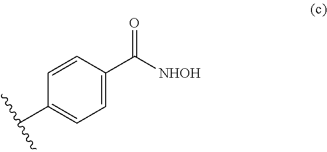

Also provided is a compound of formula (II):

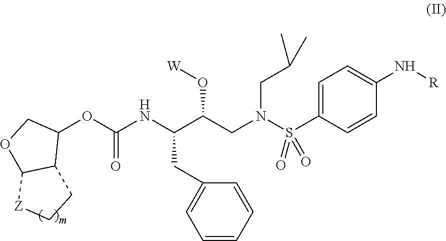

wherein W is H, deuterium, or PO₃H;
Z is CH₂ or a heteroatom selected from O, S, and N;
m is an integer from 0 to 2; and
R is selected from the group consisting of (a), (b), (c) and (d):

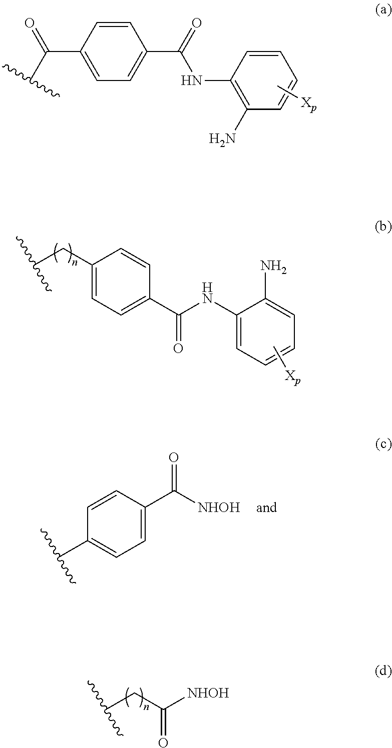

wherein Xₚ is halogen, wherein p is an integer from 1 to 4; and
n is an integer from 0 to 6;
or a pharmaceutically acceptable salt, hydrate, tautomer, or optical isomer thereof.

In some embodiments of the compound of formula (II), X is fluorine.

In some embodiments, the compound of formula (I) is:

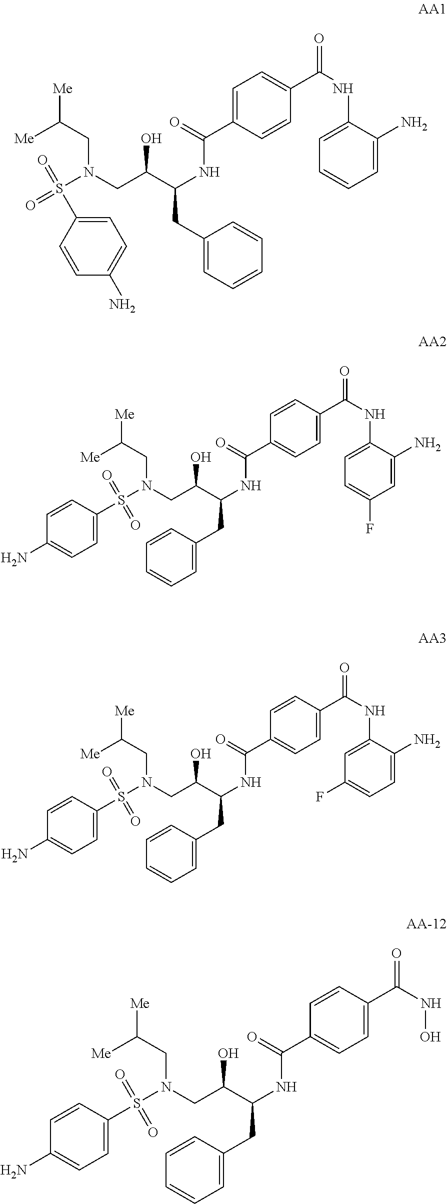 [SMILES: CC(C)CN(C[C@@H](O)[C@H](Cc1ccccc1)NC(=O)c1ccc(C(=O)Nc2ccccc2N)cc1)S(=O)(=O)c1ccc(N)cc1 ; CC(C)CN(C[C@@H](O)[C@H](Cc1ccccc1)NC(=O)c1ccc(C(=O)Nc2ccc(F)cc2N)cc1)S(=O)(=O)c1ccc(N)cc1 ; CC(C)CN(C[C@@H](O)[C@H](Cc1ccccc1)NC(=O)c1ccc(C(=O)Nc2cc(F)ccc2N)cc1)S(=O)(=O)c1ccc(N)cc1 ; CC(C)CN(C[C@@H](O)[C@H](Cc1ccccc1)NC(=O)c1ccc(C(=O)NO)cc1)S(=O)(=O)c1ccc(N)cc1]

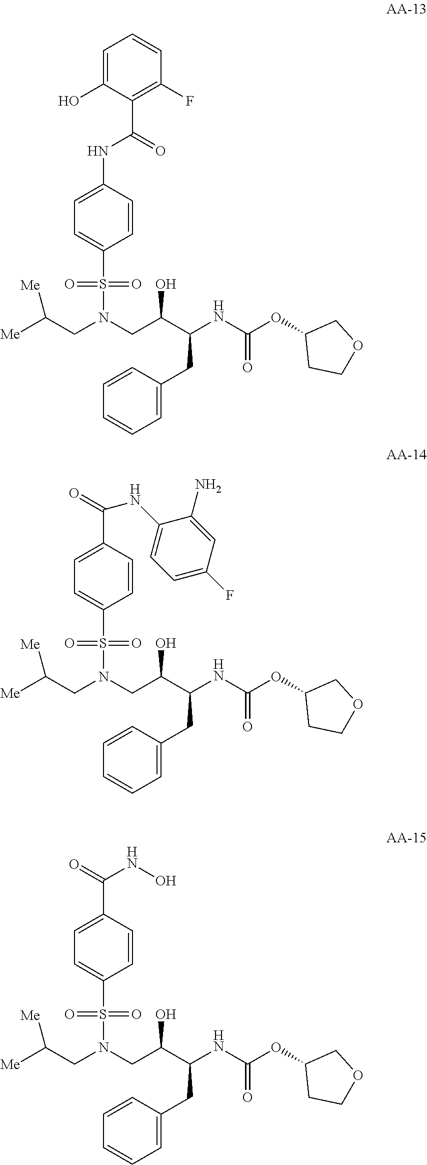 [SMILES: CC(C)CN(C[C@@H](O)[C@H](Cc1ccccc1)NC(=O)O[C@H]1CCOC1)S(=O)(=O)c1ccc(NC(=O)c2c(O)cccc2F)cc1 ; CC(C)CN(C[C@@H](O)[C@H](Cc1ccccc1)NC(=O)O[C@H]1CCOC1)S(=O)(=O)c1ccc(C(=O)Nc2ccc(F)cc2N)cc1 ; CC(C)CN(C[C@@H](O)[C@H](Cc1ccccc1)NC(=O)O[C@H]1CCOC1)S(=O)(=O)c1ccc(C(=O)NO)cc1]

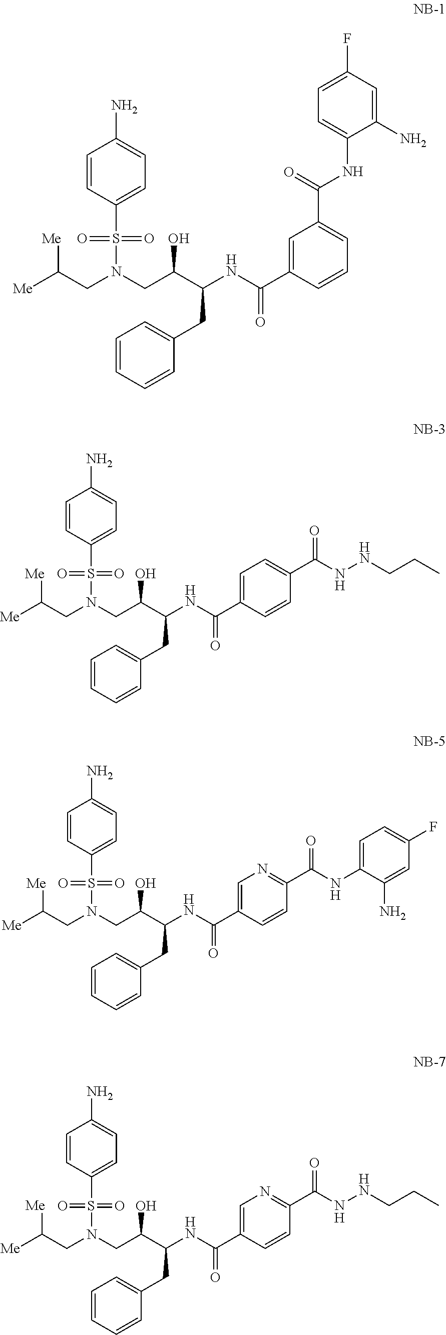 [SMILES: CC(C)CN(C[C@@H](O)[C@H](Cc1ccccc1)NC(=O)c1cccc(C(=O)Nc2ccc(F)cc2N)c1)S(=O)(=O)c1ccc(N)cc1 ; CCCNNC(=O)c1ccc(C(=O)N[C@@H](Cc2ccccc2)[C@H](O)CN(CC(C)C)S(=O)(=O)c2ccc(N)cc2)cc1 ; CC(C)CN(C[C@@H](O)[C@H](Cc1ccccc1)NC(=O)c1ccc(C(=O)Nc2ccc(F)cc2N)nc1)S(=O)(=O)c1ccc(N)cc1 ; CCCNNC(=O)c1ccc(C(=O)N[C@@H](Cc2ccccc2)[C@H](O)CN(CC(C)C)S(=O)(=O)c2ccc(N)cc2)cn1]

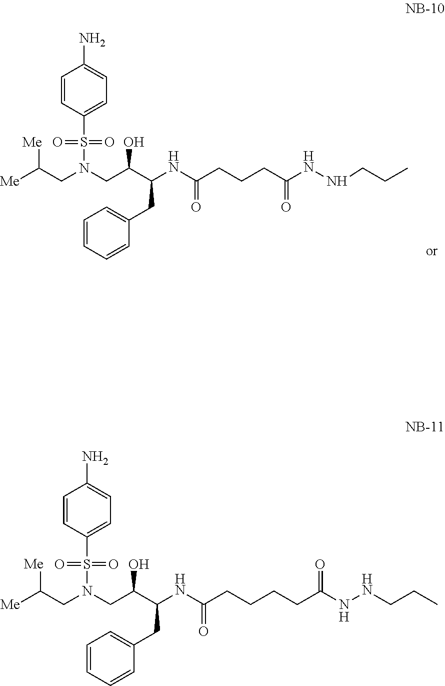 [SMILES: CCCNNC(=O)CCCC(=O)N[C@@H](Cc1ccccc1)[C@H](O)CN(CC(C)C)S(=O)(=O)c1ccc(N)cc1 ; CCCNNC(=O)CCCCC(=O)N[C@@H](Cc1ccccc1)[C@H](O)CN(CC(C)C)S(=O)(=O)c1ccc(N)cc1]

or a pharmaceutically acceptable salt, hydrate, tautomer, or optical isomer thereof.

In some embodiments, the compound of formula (IA) is:

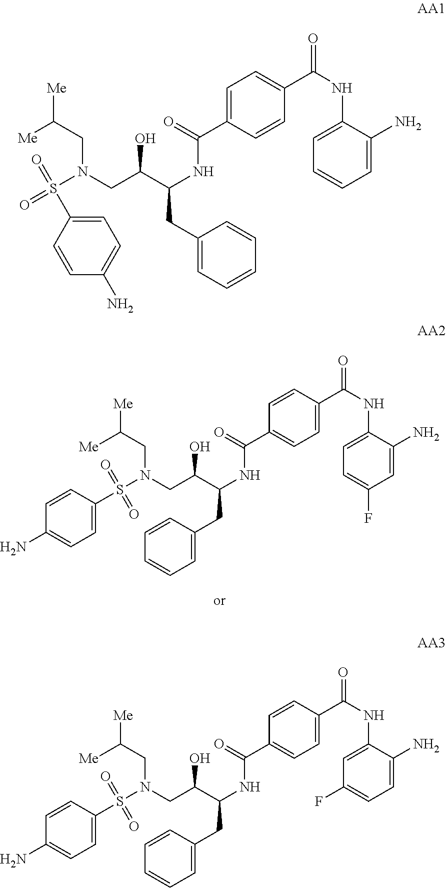 [SMILES: CC(C)CN(C[C@@H](O)[C@H](Cc1ccccc1)NC(=O)c1ccc(C(=O)Nc2ccccc2N)cc1)S(=O)(=O)c1ccc(N)cc1 ; CC(C)CN(C[C@@H](O)[C@H](Cc1ccccc1)NC(=O)c1ccc(C(=O)Nc2ccc(F)cc2N)cc1)S(=O)(=O)c1ccc(N)cc1 ; CC(C)CN(C[C@@H](O)[C@H](Cc1ccccc1)NC(=O)c1ccc(C(=O)Nc2cc(F)ccc2N)cc1)S(=O)(=O)c1ccc(N)cc1]

or a pharmaceutically acceptable salt, hydrate, tautomer, or optical isomer thereof.

Provided is a pharmaceutical composition comprising a compound of formula (I, or a pharmaceutically acceptable salt, hydrate, tautomer, or optical isomer thereof, and a pharmaceutically acceptable carrier, excipient, or diluent.

Provided is a pharmaceutical composition comprising a compound of formula (II), or a pharmaceutically acceptable salt, hydrate, tautomer, or optical isomer thereof, and a pharmaceutically acceptable carrier, excipient, or diluent.

Further provided is a method of dual inhibition of histone deacetylase 3 (HDAC3) and human immunodeficiency virus type-1 (HIV-1) protease (PR) in a patient in need thereof, which method comprises administering to the patient an inhibitory amount of (i) a compound of formula (I) or (II), (ii) a pharmaceutically acceptable salt, hydrate, tautomer, or optical isomer of (i), or (iii) a pharmaceutical composition comprising (i) or (ii) and a pharmaceutically acceptable carrier, excipient, or diluent, whereupon HDAC3 and HIV-1 PR in the patient are inhibited. In some embodiments of the method, HIV-1 infection is inhibited. In some embodiments of the method, HIV-1 infection is treated. In some embodiments of the method, HIV-1 infection is eradicated.

still further provided is a method of inhibiting cell-to-cell transmission of HIV-1 in a patient in need thereof, which method comprises administering to the patient an inhibitory amount of a compound of formula (I) or (II), or a pharmaceutically acceptable salt, hydrate, tautomer, or optical isomer thereof, which inhibits cell-to-cell transmission of HIV-1, or a pharmaceutical composition comprising the same and a pharmaceutically acceptable carrier, excipient, or diluent, whereupon cell-to-cell transmission of HIV-1 in the patient is inhibited.

In some embodiments of the method of inhibiting cell-to-cell transmission of HIV-1, the compound of formula (I) is a compound wherein
R₂ is

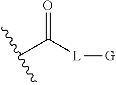

wherein L is C₁-C₅ alkyl, C₆-C₁₂ aryl, or C₅-C₁₂ heteroaryl; and
G is

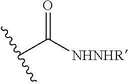

wherein R′ is C₁-C₅ alkyl.

In some embodiments of the method of inhibiting cell-to-cell transmission of HIV-1, the compound of formula (I) is selected from:

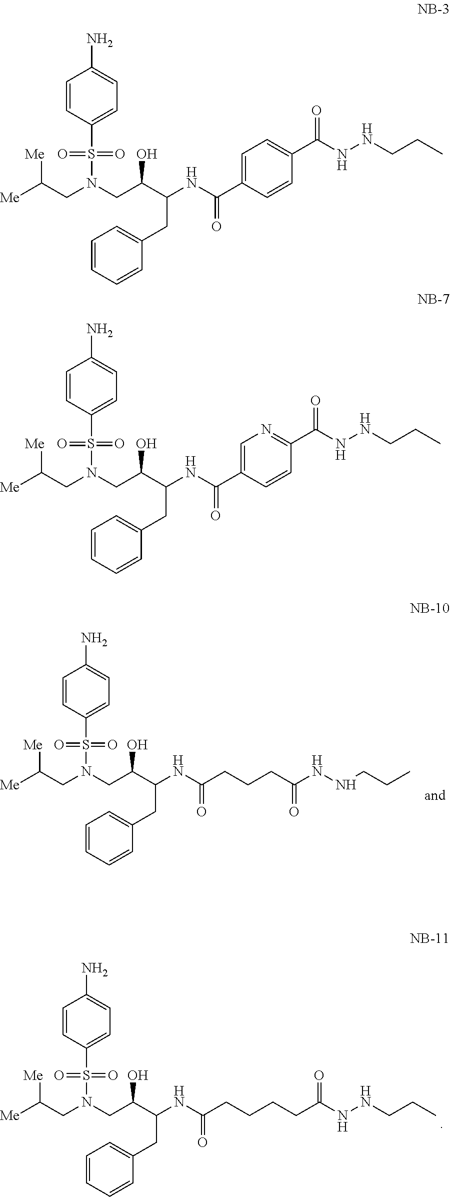 [SMILES: CCCNNC(=O)c1ccc(C(=O)NC(Cc2ccccc2)[C@H](O)CN(CC(C)C)S(=O)(=O)c2ccc(N)cc2)cc1 ; CCCNNC(=O)c1ccc(C(=O)NC(Cc2ccccc2)[C@H](O)CN(CC(C)C)S(=O)(=O)c2ccc(N)cc2)cn1 ; CCCNNC(=O)CCCC(=O)NC(Cc1ccccc1)[C@H](O)CN(CC(C)C)S(=O)(=O)c1ccc(N)cc1 ; CCCNNC(=O)CCCCC(=O)NC(Cc1ccccc1)[C@H](O)CN(CC(C)C)S(=O)(=O)c1ccc(N)cc1]

### BRIEF DESCRIPTION OF THE DRAWINGS

The present disclosure will be more readily understood from the detailed description of embodiments presented below considered in conjunction with the attached drawings of which:

FIG. 1 shows the schematic of the “shock and kill” approach using histone deacetylase (HDAC) inhibitors to bring human immunodeficiency virus 1 (HIV-1) out of latency and combined antiretroviral therapy (cART) drugs, such as HIV-1 protease (PR) inhibitors, to eliminate released virions.

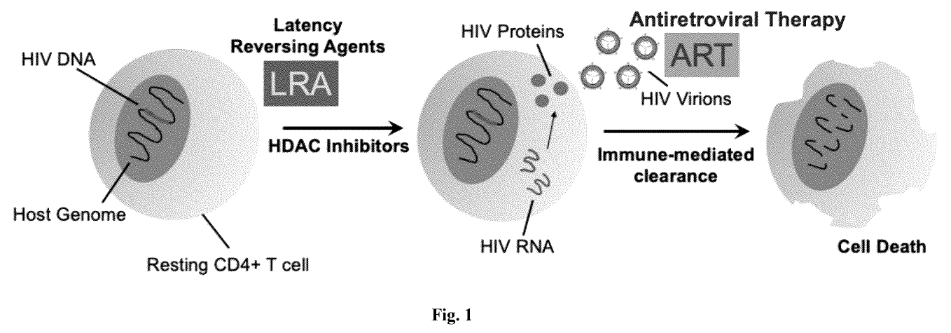

FIG. 2 shows provirus activation in HIV-1 in latently infected cells. ACH-2 cells, a human T cell line latently infected with the HIV-1LAV strain, were used to test the effects of compound AA2 on provirus activation. Cells were treated with varying concentrations of compound AA2 and DMSO as a negative control. ACH-2 cells were subjected to intracellular staining of HIV-24 to determine provirus activation. The data demonstrate that compound AA2 robustly activated HIV-1 in latently infected cells in a concentration-dependent manner.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The term “HDAC3/HIV-1 dual inhibitor” refers to a compound that inhibits both histone deacetylase 3 (HDAC3, also called RPD3-2, HD3, KDAC3, RPD3, protein deacetylase HDAC3, protein deacylase HDAC3, EC 3.1.3.98 and SMAP45) and human immunodeficiency virus type-1 (HIV-1) protease (PR).

Provided is a compound of formula (I):

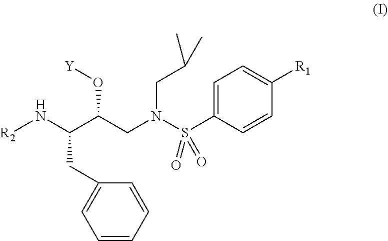

wherein Y is H, deuterium, or PO₃H;
R₁ is NH₂ or a group selected from:

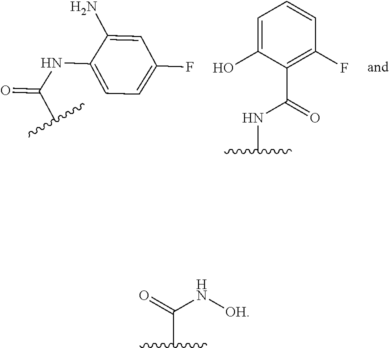

and
R₂ is

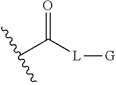

wherein L is O, C₁-C₅ alkyl, C₆-C₁₂ aryl, or C₅-C₁₂ heteroaryl; and
G is selected from the group consisting of (i), (ii), (iii), (iv), and (v):

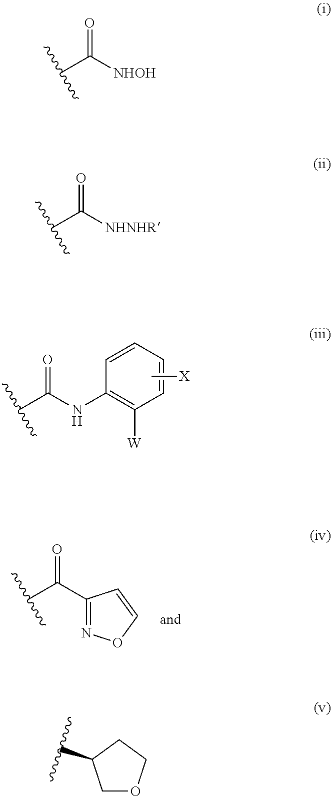

wherein R′ is C₁-C₅ alkyl;
X is H or halogen; and
W is OH, NH₂, or SCH₃;
with the proviso that when G is (v), R₁ is not NH₂,
or a pharmaceutically acceptable salt, hydrate, tautomer, or optical isomer thereof.

In some embodiments of the compound of formula (I), R₁ is selected from:

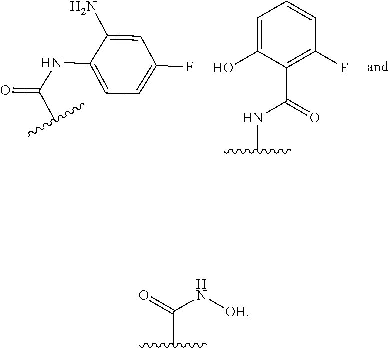

In some embodiments of the compound of formula (I), R₁ is NH₂;
and R₂ is

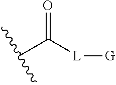

wherein L is o, C₁-C₅ alkyl, C₆-C₁₂ aryl, or C₅-C₁₂ heteroaryl; and
G is selected from the group consisting of (i), (ii), (iii), and (iv):

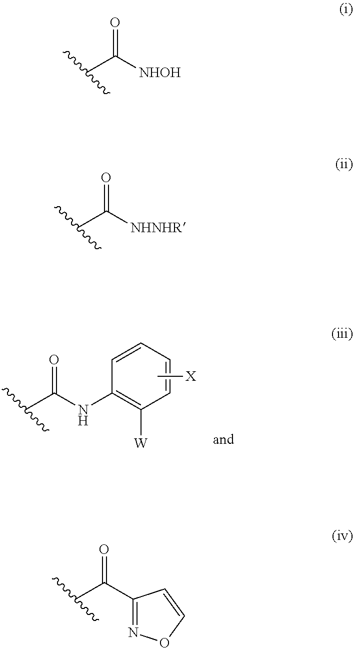

wherein R′ is C₁-C₅ alkyl;
X is H or halogen; and
W is OH, NH₂, or SCH₃.

In some embodiments of the compound of formula (I), R₂ is

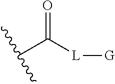

wherein L is O;
and G is

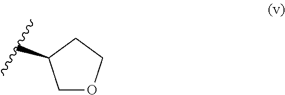 [SMILES: *[C@H]1CCOC1]

with the proviso that when G is (v), R₁ is not NH₂.

In some embodiments of the compound of formula (I), R₂ is

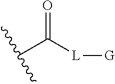

wherein L is C₆-C₁₂ aryl; and G is selected from the group consisting of (i), (ii), and (iii):

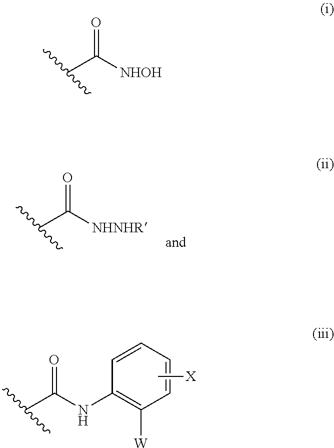

wherein
R′ is C₁-C₅ alkyl;
X is H or halogen; and
W is NH₂.

In some embodiments of the compound of formula (I), L is phenyl.

In some embodiments of the compound of formula (I), R₂ is

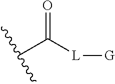

wherein L is C₅-C₁₂ heteroaryl; and G is selected from the group consisting of (i), (ii), and (iii):

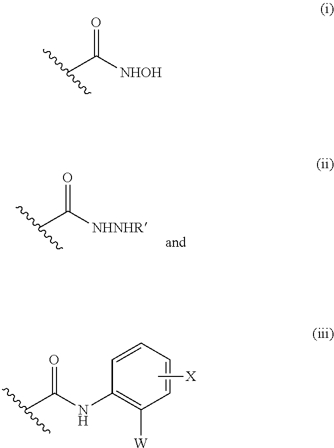

wherein
R′ is C₁-C₅ alkyl;
X is H or halogen; and
W is NH₂.

In some embodiments of the compound of formula (I), L is pyridyl.

In some embodiments of the compound of formula (I), R₂ is

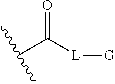

wherein L is pyridyl; and G is (iii):

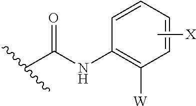

wherein
X is H or halogen; and
W is NH₂.

In some embodiments of the compound of formula (I), Y is H.

In some embodiments of the compound of formula (I), R₁ is NH₂;
and R₂ is

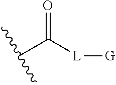

wherein L is C₁-C₅ alkyl, C₆-C₁₂ aryl, or C₅-C₁₂ heteroaryl; and
Gs

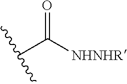

wherein R′ is C₁-C₅ alkyl.

In some embodiments of the compound of formula (I), R₁ is NH₂;
and R₂ is

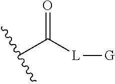

wherein L is C₁-C₅ alkyl, phenyl, or pyridyl; and
G is

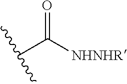

wherein R′ is C₁-C₅ alkyl.

In some embodiments, the compound of formula (I) is a compound of formula (IA):

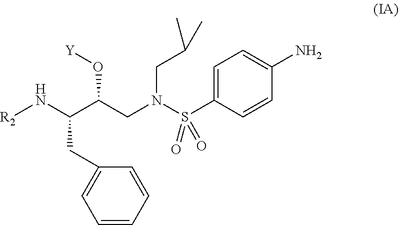

wherein Y is H, deuterium, or PO₃H; and
R is selected from the group consisting of (a), (b), (c) and (d):

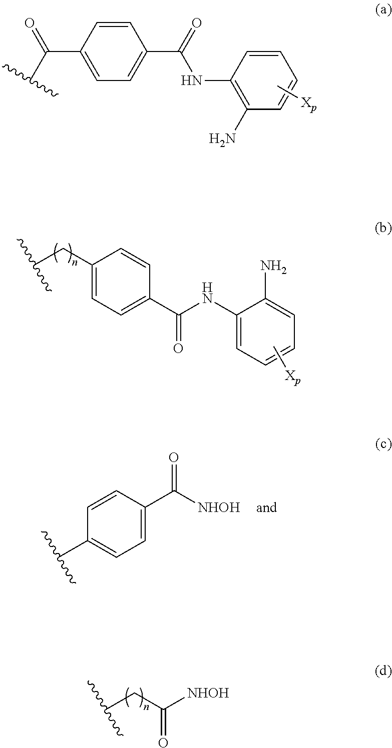

wherein Xₚ is halogen, wherein p is an integer from 1 to 4; and
n is an integer from 0 to 6;
or a pharmaceutically acceptable salt, hydrate, tautomer, or optical isomer thereof.

In some embodiments of the compound of formula (IA), R is:

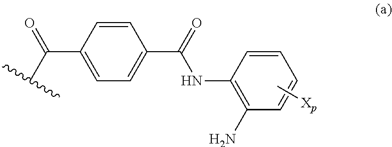

wherein Xₚ is as defined above.

In some embodiments of the compound of formula (IA), R is:

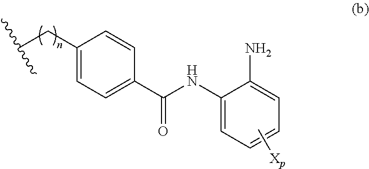

wherein Xₚ and n are as defined above.

In some embodiments of the compound of formula (IA), R is:

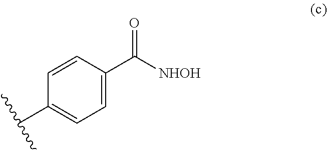

In some embodiments of the compound of formula (IA), X is fluorine.

In some embodiments of the compound of formula (IA), Y is H.

Also provided is a compound of formula (II):

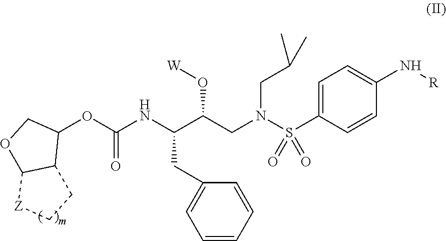

wherein W is H, deuterium, or PO₃H;
Z is CH₂ or a heteroatom selected from O, S, and N;
m is an integer from 0 to 2; and
R is selected from the group consisting of (a), (b), (c) and (d):

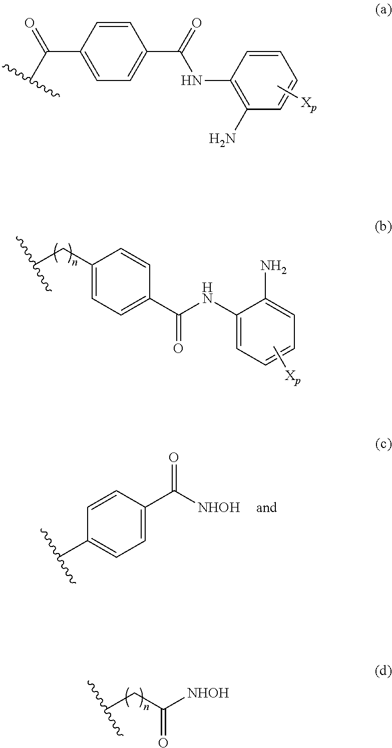

wherein Xₚ is halogen, wherein p is an integer from 1 to 4; and
n is an integer from 0 to 6;
or a pharmaceutically acceptable salt, hydrate, tautomer, or optical isomer thereof.

In some embodiments of the compound of formula (II), X is fluorine.

In some embodiments of the compound of formula (II), W is H.

In some embodiments, the compound of formula (IA) is:

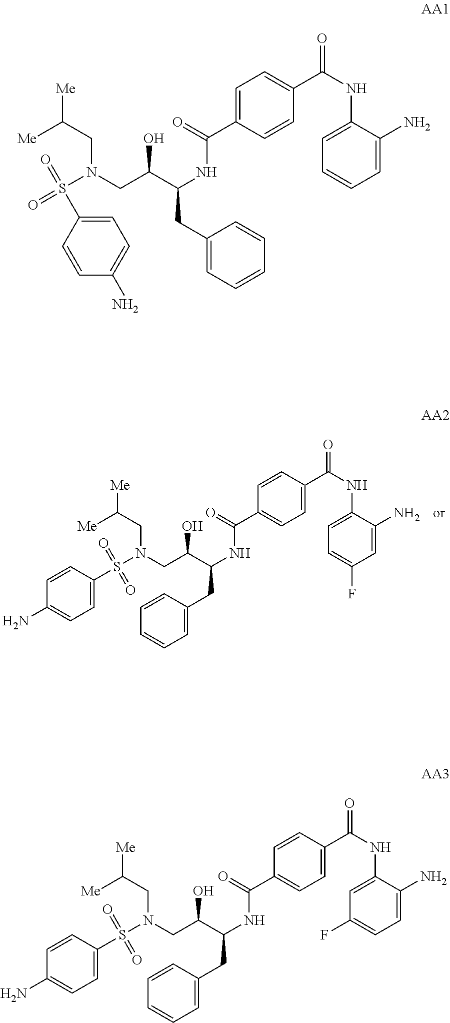 [SMILES: CC(C)CN(C[C@@H](O)[C@H](Cc1ccccc1)NC(=O)c1ccc(C(=O)Nc2ccccc2N)cc1)S(=O)(=O)c1ccc(N)cc1 ; CC(C)CN(C[C@@H](O)[C@H](Cc1ccccc1)NC(=O)c1ccc(C(=O)Nc2ccc(F)cc2N)cc1)S(=O)(=O)c1ccc(N)cc1 ; CC(C)CN(C[C@@H](O)[C@H](Cc1ccccc1)NC(=O)c1ccc(C(=O)Nc2cc(F)ccc2N)cc1)S(=O)(=O)c1ccc(N)cc1]

or a pharmaceutically acceptable salt, hydrate, tautomer, or optical isomer thereof.

In some embodiments, the compound of formula (I) is:

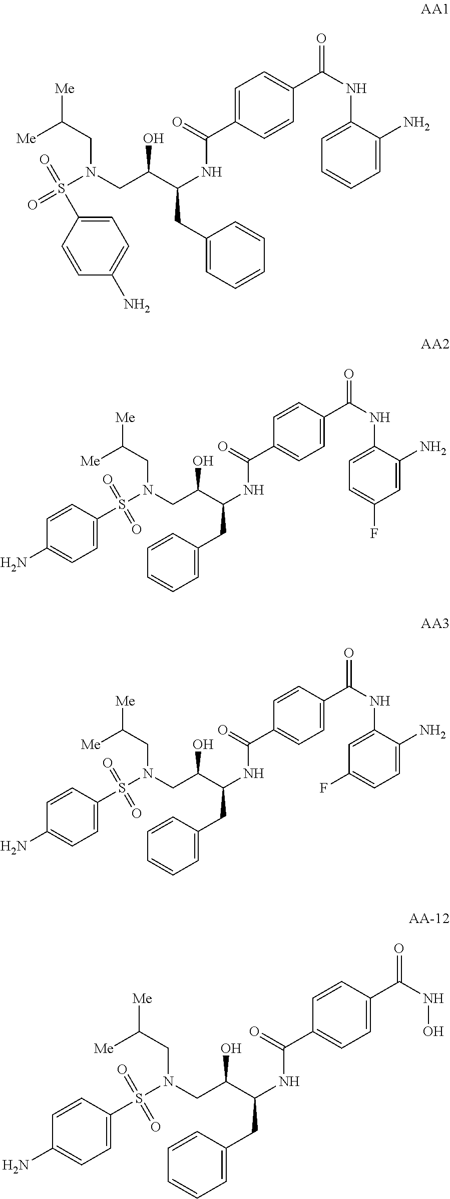 [SMILES: CC(C)CN(C[C@@H](O)[C@H](Cc1ccccc1)NC(=O)c1ccc(C(=O)Nc2ccccc2N)cc1)S(=O)(=O)c1ccc(N)cc1 ; CC(C)CN(C[C@@H](O)[C@H](Cc1ccccc1)NC(=O)c1ccc(C(=O)Nc2ccc(F)cc2N)cc1)S(=O)(=O)c1ccc(N)cc1 ; CC(C)CN(C[C@@H](O)[C@H](Cc1ccccc1)NC(=O)c1ccc(C(=O)Nc2cc(F)ccc2N)cc1)S(=O)(=O)c1ccc(N)cc1 ; CC(C)CN(C[C@@H](O)[C@H](Cc1ccccc1)NC(=O)c1ccc(C(=O)NO)cc1)S(=O)(=O)c1ccc(N)cc1]

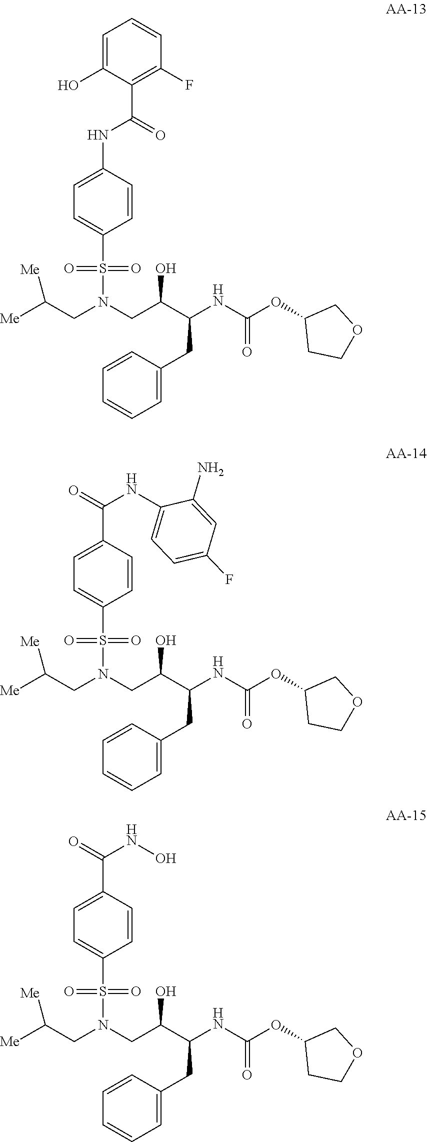 [SMILES: CC(C)CN(C[C@@H](O)[C@H](Cc1ccccc1)NC(=O)O[C@H]1CCOC1)S(=O)(=O)c1ccc(NC(=O)c2c(O)cccc2F)cc1 ; CC(C)CN(C[C@@H](O)[C@H](Cc1ccccc1)NC(=O)O[C@H]1CCOC1)S(=O)(=O)c1ccc(C(=O)Nc2ccc(F)cc2N)cc1 ; CC(C)CN(C[C@@H](O)[C@H](Cc1ccccc1)NC(=O)O[C@H]1CCOC1)S(=O)(=O)c1ccc(C(=O)NO)cc1]

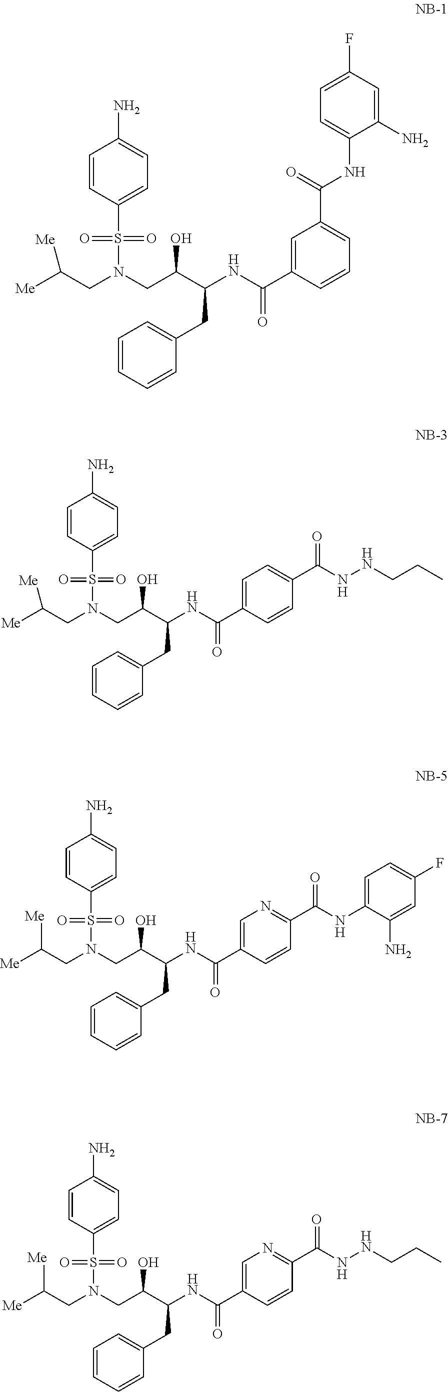 [SMILES: CC(C)CN(C[C@@H](O)[C@H](Cc1ccccc1)NC(=O)c1cccc(C(=O)Nc2ccc(F)cc2N)c1)S(=O)(=O)c1ccc(N)cc1 ; CCCNNC(=O)c1ccc(C(=O)N[C@@H](Cc2ccccc2)[C@H](O)CN(CC(C)C)S(=O)(=O)c2ccc(N)cc2)cc1 ; CC(C)CN(C[C@@H](O)[C@H](Cc1ccccc1)NC(=O)c1ccc(C(=O)Nc2ccc(F)cc2N)nc1)S(=O)(=O)c1ccc(N)cc1 ; CCCNNC(=O)c1ccc(C(=O)N[C@@H](Cc2ccccc2)[C@H](O)CN(CC(C)C)S(=O)(=O)c2ccc(N)cc2)cn1]

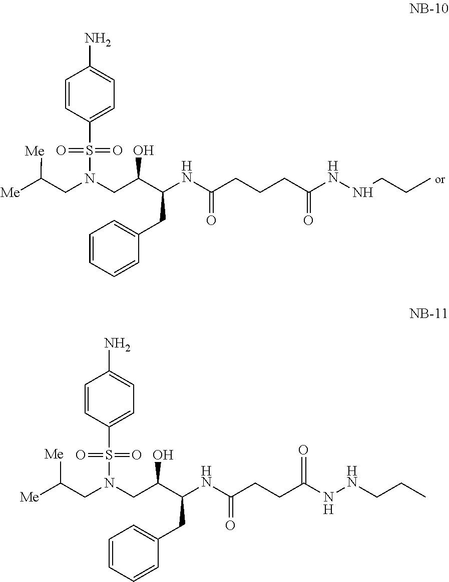 [SMILES: CCCNNC(=O)CCCC(=O)N[C@@H](Cc1ccccc1)[C@H](O)CN(CC(C)C)S(=O)(=O)c1ccc(N)cc1 ; CCCNNC(=O)CCC(=O)N[C@@H](Cc1ccccc1)[C@H](O)CN(CC(C)C)S(=O)(=O)c1ccc(N)cc1]

or a pharmaceutically acceptable salt, hydrate, tautomer, or optical isomer thereof.

In some embodiments, the compounds of formula (I), (IA), and (II) are dual inhibitors of HDAC3 and HIV-1 PR. HDAC3 inhibition can activate proviruses in latently-infected cells, and HIV-1 PR inhibition can prevent new infections by simultaneously killing virions newly released from HDAC3 inhibitor provirus-activated latently-infected cells. The compounds can be used for the treatment and eradication of HIV-1 infection. The compounds contain a hydroxyethylene isostere unit and a zinc-binding moiety. In some embodiments, the compounds can inhibit cell-to-cell transmission of HIV-1 infection. In some embodiments, the compounds, which inhibit cell-to-cell transmission of HIV-1 infection, comprise a R₂ group wherein G is

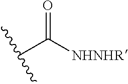

wherein R′ is C₁-C₅ alkyl. Examples of such compounds include, but are not limited to,

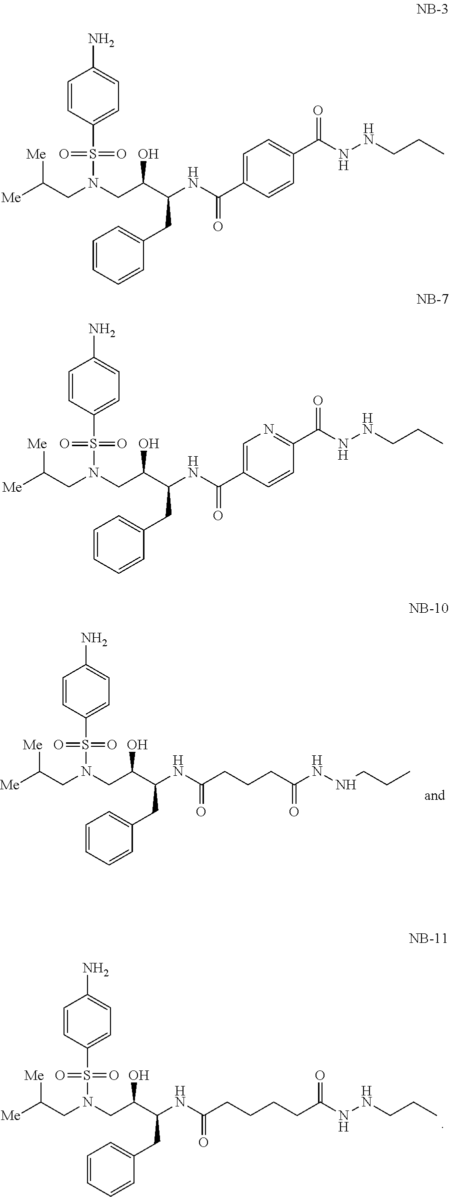 [SMILES: CCCNNC(=O)c1ccc(C(=O)N[C@@H](Cc2ccccc2)[C@H](O)CN(CC(C)C)S(=O)(=O)c2ccc(N)cc2)cc1 ; CCCNNC(=O)c1ccc(C(=O)N[C@@H](Cc2ccccc2)[C@H](O)CN(CC(C)C)S(=O)(=O)c2ccc(N)cc2)cn1 ; CCCNNC(=O)CCCC(=O)N[C@@H](Cc1ccccc1)[C@H](O)CN(CC(C)C)S(=O)(=O)c1ccc(N)cc1 ; CCCNNC(=O)CCCCC(=O)N[C@@H](Cc1ccccc1)[C@H](O)CN(CC(C)C)S(=O)(=O)c1ccc(N)cc1]

In some embodiments, the degrees to which the compound inhibits HDAC3 and HIV-1 PR can be the same. In other embodiments, the compound inhibits HDAC3 to a greater extent than it inhibits HIV-1 PR. In yet other embodiments, the compound inhibits HIV-1 PR to a greater extent than it inhibits HDAC3. It is preferable, and even desirable, for the compound to inhibit HDAC3 and HIV-1 PR to at least approximately the same extent.

The term “substituted” refers to a functional group in which one or more hydrogen atoms contained therein are replaced by one or more non-hydrogen atoms. The term “functional group” or “substituent” refers to a group that can be, or is, substituted onto a molecule. Examples of substituents or functional groups include, but are not limited to, a halo (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

Non-limiting examples of substituents, which can be bonded to a substituted carbon atom (or other atom, such as nitrogen), include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, (CH₂)₀₋₂P(O)OR₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R wherein R can be hydrogen or a carbon-based moiety, and wherein the carbon-based moiety can itself be further substituted; for example, where R can be hydrogen, alkyl, acyl, cycloalkyl, aryl, aralkyl, heterocyclyl, heteroaryl, or heteroarylalkyl, any alkyl, acyl, cycloalkyl, aryl, aralkyl, heterocyclyl, heteroaryl, or heteroarylalkyl or R can be independently mono- or multi-substituted; or when two R groups bonded to a nitrogen atom or to adjacent nitrogen atoms can, together with the nitrogen atom or atoms to which they are bonded, form a heterocyclyl, the heterocycle can be mono- or independently multi-substituted.

The terms “optionally substituted” and “optional substituents” indicate that the groups in question are either unsubstituted or substituted with one or more of the substituents specified. When the groups in question are substituted with more than one substituent, the substituents may be the same or different. When used with the terms “independently,” “independently are,” and “independently selected from,” the groups in question may be the same or different. Certain of the herein defined terms may occur more than once in the structure and, upon such occurrence, each term shall be defined independently of the other.

The term “halogen” refers to an atom selected from fluorine, chlorine, bromine, and iodine.

The term “alkyl” refers to substituted or unsubstituted straight-chain and branched alkyl groups and cycloalkyl groups having from 1 to about 20 carbon atoms (e.g., C₁-C₂₀), 1 to 12 carbons (e.g., C₁-C₁₂), 1 to 8 carbon atoms (e.g., C₁-C₈), or, in some embodiments, from 1 to 6 carbon atoms (e.g., C₁-C₆). Examples of straight-chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “aryl” refers to substituted and unsubstituted cyclic aromatic hydrocarbons that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (e.g., C₆-C₁₄) or from 6 to 10 carbon atoms (e.g., C₆-C₁₀) in the ring portions of the groups. Aryl groups can be unsubstituted or substituted, as defined herein. Representative substituted aryl groups can be mono-substituted or substituted more than once, such as, but not limited to, a phenyl substituted with 2-, 3-, 4-, 5-, or 6-substituents or 2-8 substituted naphthyl groups, which can be substituted with carbon or non-carbon groups such as those listed herein.

A heteroaryl ring is an embodiment of a heterocyclyl group. The phrase “heterocyclyl group” includes fused ring species including those that include fused aromatic and non-aromatic groups. Representative heterocyclyl groups include, but are not limited to, pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “compound” refers to include all stereoisomers, geometric isomers, and tautomers of the structures depicted. The compounds described herein as “optical isomers” may contain one or more chiral centers or may otherwise be capable of existing as multiple stereoisomers. In various embodiments, the compounds are not limited to any particular stereochemical requirement, and the compounds, and compositions, methods, uses, and medicaments that include them, may be optically pure or any of a variety of stereoisomeric mixtures, including racemic and other mixtures of enantiomers, other mixtures of diastereomers, and the like. Such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. In various embodiments, the compounds are not limited to any particular geometric isomer requirement, and the compounds, and compositions, methods, uses, and medicaments that include them, may be pure or any of a variety of geometric isomer mixtures. Such mixtures of geometric isomers may include a single configuration at one or more double bonds, while including mixtures of geometry at one or more other double bonds.

The term “solvate” refers to a combination, physical association, and/or solvation of a compound described herein with a solvent molecule such as, e.g., a disolvate, monosolvate, or hemisolvate, where the ratio of the solvent molecule to a compound described is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates.

Solvated forms of the compounds can be prepared with a pharmaceutically acceptable solvent. Examples of the solvents include, but are not limited to, water, methanol, and ethanol, and it is intended that the disclosure includes both solvated and unsolvated forms of above-described compounds. One type of solvate is a hydrate. A “hydrate” relates to a particular subgroup of solvates where the solvent molecule is water. Solvates typically can function as pharmacological equivalents. The preparation of solvates is known in the art. For example, M. Caira et al., J. Pharmaceut. Sci., 2004, 93(3): 601-611, describes the preparation of solvates of fluconazole with ethyl acetate and with water. Similar preparation of solvates, hemisolvates, hydrates, and the like are described by E. C. van Tonder et al., AAPS Pharm. Sci. Tech., 2004, 5(1): Article 12, and A. L. Bingham et al, 200, Chem. Commun., 603-604. A typical, non-limiting, process of preparing a solvate would involve dissolving a compound in a desired solvent (organic, water, or a mixture thereof) at temperatures above 20° C. to about 25° C., then cooling the solution at a rate sufficient to form crystals, and isolating the crystals by known methods, e.g., filtration. Analytical techniques, such as infrared spectroscopy, can be used to confirm the presence of the solvent in a crystal of the solvate.

The term “pharmaceutically acceptable salt” refers to salts or zwitterionic forms of compounds described herein. Examples of the salts of the compounds described herein include, but are not limited to, hydrochloride salt, hydrobromide salt, hydroiodide salt, sulfate salt, bisulfate salt, 2-hydroxyethansulfonate salt, phosphate salt, hydrogen phosphate salt, acetate salt, adipate salt, alginate salt, aspartate salt, benzoate salt, bisulfate salt, butyrate salt, camphorate salt, camphorsulfonate salt, digluconate salt, glycerolphosphate salt, hemisulfate salt, heptanoate salt, hexanoate salt, formate salt, succinate salt, fumarate salt, maleate salt, ascorbate salt, isethionate salt, salicylate salt, methanesulfonate salt, mesitylenesulfonate salt, naphthylenesulfonate salt, nicotinate salt, 2-naphthalenesulfonate salt, oxalate salt, pamoate salt, pectinate salt, persulfate salt, 3-phenylproprionate salt, picrate salt, pivalate salt, propionate salt, trichloroacetate salt, trifluoroacetate salt, phosphate salt, glutamate salt, bicarbonate salt, paratoluenesulfonate salt, undecanoate salt, lactate salt, citrate salt, tartrate salt, gluconate salt, methanesulfonate salt, ethanedisulfonate salt, benzene sulfonate salt, and p-toluenesulfonate salt.

Any reference compounds are intended to include pharmaceutically acceptable salts and hydrates thereof.

Provided is a pharmaceutical composition comprising a compound of formula (I), or a pharmaceutically acceptable salt, hydrate, tautomer, or optical isomer thereof, and a pharmaceutically acceptable carrier, excipient, or diluent.

Provided is a pharmaceutical composition comprising a compound of formula (II), or a pharmaceutically acceptable salt, hydrate, tautomer, or optical isomer thereof, and a pharmaceutically acceptable carrier, excipient, or diluent.

Any suitable carrier, excipient, or diluent, as known in the art, can be used. The carrier, excipient, or diluent can vary based on the particular route of administration (see, e.g., Remington's The Science and Practice of Pharmacy, 23^{rd} ed. (2020)).

Compounds of formula (I) and (II) are dual inhibitors of HDAC3 and HIV-1 PR and are useful in the treatment of a disease or condition wherein inhibition of HDAC3 and HIV-1 PR provides a benefit.

Provided is a method of dual inhibition of histone deacetylase 3 (HDAC3) and human immunodeficiency virus type-1 (HIV-1) protease (PR) in a patient in need thereof, which method comprises administering to the patient an inhibitory amount of (i) a compound of formula (I) or (II), (ii) a pharmaceutically acceptable salt, hydrate, tautomer, or optical isomer of (i), (iii) a pharmaceutical composition comprising (i) or (ii) and a pharmaceutically acceptable carrier, excipient, or diluent, whereupon HDAC3 and HIV-1 PR in the patient are inhibited. In some embodiments of the method, HIV-1 infection is inhibited. In some embodiments of the method, HIV-1 infection is treated. In some embodiments of the method, HIV-1 infection is eradicated.

Also provided is a method of inhibiting cell-to-cell transmission of HIV-1 in a patient in need thereof, which method comprises administering to the patient an inhibitory amount of a compound of formula (I) or (II), or a pharmaceutically acceptable salt, hydrate, tautomer, or optical isomer thereof, which inhibits cell-to-cell transmission of HIV-1, or a pharmaceutical composition comprising the same and a pharmaceutically acceptable carrier, excipient, or diluent, whereupon cell-to-cell transmission of HIV-1 in the patient is inhibited.

In some embodiments, the compounds inhibit both HDAC3 and HIV-1 PR simultaneously. Any suitable route can be used to administer the compound of formula (I) or (II). The compounds can be administered simultaneously or sequentially, in either order, by the same or different routes.

The compounds can also be used in combination with other compounds or known drugs that are used as HIV-1 inhibitors or antiviral agents. In some embodiments, a pharmaceutical composition further comprises at least one additional pharmaceutically active agent.

Pharmaceutical compositions can be prepared by combining a compound of formula (I) or (II), or a pharmaceutically acceptable salt, solvate, tautomer, or stereoisomer of either, with a pharmaceutically acceptable carrier or excipient and, optionally, one or more additional pharmaceutically active agents. One or more additional pharmaceutically active agents can be known drugs that can be used to treat HIV-1 infection. Examples of known drugs that can be used to treat HIV-1 infection include, but are not limited to, Tipranavir, Atazanavir and Ritonavir. Alternatively, one or more additional pharmaceutically active agents can be administered as one or more separate pharmaceutical compositions, which can be administered simultaneously or sequentially, in any order, with one or more pharmaceutical compositions comprising a compound of formula (I), a compound of formula (II), or a combination thereof. The compounds of formula (I) and/or formula (II) can be administered in the form of pharmaceutically acceptable salts, solvates, tautomers, and/or stereoisomers. In this regard, the form of the compound of formula (I) can be the same as, or different from, the form of the compound of formula (II).

Provided is a pharmaceutical combination for treating HIV-1 infection in a patient in need thereof. The pharmaceutical combination comprises (i) a compound of formula (I) and/or a compound of formula (II), or a pharmaceutically acceptable salt, hydrate, tautomer, or optical isomer of either, (ii) an additional therapeutic agent, and (iii) optionally at least one pharmaceutically acceptable carrier, excipient, or diluent.

An additional therapeutic agent can be any known drug that can be used for treating HIV-1. Examples of known drugs that can be used to treat HIV-1 infection include, but are not limited to, tipranavir, atazanavir, ritonavir, dolutegravir, abacavir, lamivudine, elvitegravir, cobicistat, emtricitabine, tenofovir alafenamide, tenofovir disoproxil fumarate and bictegravir.

The term “pharmaceutical combination” refers to a pharmaceutical therapy resulting from the mixing or combining of more than one active ingredient. For a combination, the compound of formula (I) and/or the compound of formula (II), or a pharmaceutically acceptable salt, hydrate, tautomer, or optical isomer of either, and at least one additional therapeutic agent can be administered to a patient simultaneously or sequentially, in either order, by the same or different route of administration in a single composition or as two separate compositions to achieve the desired effect. The therapeutic agent can be administered in an amount to provide its desired therapeutic effect. The effective dosage range for each therapeutic agent is well-known in the art, and the therapeutic agent is administered to a patient in need thereof within such established ranges.

The terms “treat,” “treating,” “treated,” and “treatment” (with respect to a disease or condition) are used to describe an approach for obtaining beneficial or desired results, preferably clinical results, and include, but are not limited to, one or more of the following: improving a condition associated with a disease, curing a disease, lessening the severity of a disease, delaying progression of a disease, alleviating one or more symptoms associated with a disease, increasing the quality of life of one suffering from a disease, prolonging survival and/or prophylactic or preventative treatment.

The term “eradication” of the HIV virus means that the HIV virus is unable to replicate, the genome is deleted, fragmented, degraded, genetically inactivated, or any other physical, biological, chemical, or structural manifestation that prevents the virus from being transmissible or infecting any other cell or subject resulting in the clearance of the virus in vivo. The eradication of HIV can involve the complete elimination of HIV from the body, including the destruction of cells infected with latent HIV.

The term “pharmaceutical composition” includes an inhibitory amount of one or more compounds for treating a patient, such as a patient with HIV-1 infections, including AIDS in patient. The composition may include other components and/or ingredients, including, but not limited to, other therapeutically active compounds and/or one or more pharmaceutically acceptable carriers, diluents, excipients, and the like.

The term “pharmaceutically acceptable carrier” is art-recognized and refers to a pharmaceutically acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials, which may serve as pharmaceutically acceptable carriers, include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffered solutions; and (21) other non-toxic compatible substances employed in pharmaceutical formulations.

The term “inhibitory effect” or “inhibition” refers to the ability of a compound to reduce or block a specific biological or chemical activity, enzyme, or receptor, thereby preventing, suppressing, or altering its normal function.

The term “inhibitory amount” means any amount of a compound or a pharmaceutical composition comprising the compound that is sufficient to achieve an “inhibitor effect” or “inhibition”. The term “therapeutically effective amount” means the amount of a compound that is effective to treat a disease or a disorder, such as HIV-1 infections including AIDS, at a reasonable benefit/risk ratio. The therapeutically effective amount of such compound will vary depending upon the patient and the disease or disorder being treated, the weight and age of the patient, the severity of the disease or disorder, the manner of administration, and the like, which can readily be determined by one of skill in the art.

The compounds can be administered in unit dosage forms and/or compositions containing one or more pharmaceutically acceptable carriers, adjuvants, diluents, excipients, and/or vehicles, and combinations thereof. As used herein, the term “administering” and its formatives generally refer to any and all means of introducing compounds to the patient including, but not limited to, by oral, intravenous, intratumoral, intramuscular, subcutaneous, transdermal, topical, and like routes of administration.

For oral administration, the compounds can be formulated readily by combining the active compound(s) with pharmaceutically acceptable carriers, excipients, or diluents well-known in the art. Such carriers, excipients, or diluents enable the compounds to be formulated as tablets, pills, powders, dragees, capsules, liquids, gels, syrups, slurries, suspensions, solutions, and the like for oral ingestion by a subject to be treated.

Useful dosages of the compounds can be determined by comparing their in vitro activity with their in vivo activity in animal models. Methods of the extrapolation of effective dosages in mice and other animals to human subjects are known in the art. Indeed, the dosage of the compounds can vary significantly depending on the condition of the subject, the age of the subject, the type of disease the subject is experiencing or at risk of experiencing, the particular compounds used, how advanced the pathology is, the route of administration of the compounds and the possibility of co-usage of other therapeutic treatments or additional drugs in combination therapies. The amount of the composition required for use in inhibition (e.g., the inhibitory amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise.

The compositions comprising the compound (s) can be formulated in a unit dosage form, each dosage containing from about 5 to about 1,000 mg (1 g), more usually about 100 mg to about 500 mg, of the compound, i.e., active ingredient. In some embodiments, the compositions can contain from about 5 mg to about 50 mg of the active ingredient. One having ordinary skill in the art will appreciate that this embodies compounds or compositions containing about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg of the active ingredient.

In some embodiments, the compositions provided herein contain from about 50 mg to about 500 mg of the active ingredient. One having ordinary skill in the art will appreciate that this embodies compounds or compositions containing about 50 mg to about 100 mg, about 100 mg to about 150 mg, about 150 mg to about 200 mg, about 200 mg to about 250 mg, about 250 mg to about 300 mg, about 350 mg to about 400 mg, or about 450 mg to about 500 mg of the active ingredient. In some embodiments, the compositions provided herein contain from about 500 mg to about 1,000 mg of the active ingredient. One having ordinary skill in the art will appreciate that this embodies compounds or compositions containing about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, or about 950 mg to about 1,000 mg of the active ingredient.

The active compound may be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

In some embodiments, the compound can be administered in an amount ranging from about 1 mg/kg to about 100 mg/kg. In some embodiments, the compound can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. For example, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg. In some embodiments, such administration can be once-daily or twice-daily (BID) administration.

The term “patient” includes human and non-human animals such as companion animals (dogs and cats and the like) and livestock animals. Livestock animals are animals raised for food production. The patient to be treated is preferably a mammal, in particular a human being.

### EXAMPLES

The following examples serve to illustrate the present disclosure. The examples are not intended to limit the scope of the claimed invention in any way.

### Design of Compounds:

To achieve dual HDAC3 and HIV-1 PR inhibition, compounds were engineered as shown below (Compounds AA1-AA3) with components of both HDAC3 and HIV-1 PR inhibitors. The resulting hybrid molecules can inhibit both targets and thus can function both to activate HIV-1 latency and prevent the production of viable HIV-1 virions. Compounds AA1-AA3 were synthesized in six steps each in about an overall yield of 25% for each compound. These compounds were evaluated for inhibition of HDAC3 and HIV-1 PR in vitro, and preliminary results indicate low μM inhibition of HDAC3 and modest HIV-1 PR activity for all three agents.

To improve the potency of these agents explored, additional zinc-binding motifs to improve HDAC3 potency, while at the same time also exploring modified placement of the HDAC component onto the HIV-1 PR module. Molecular modeling with several published crystal structures suggests that the HDAC3 component of the hybrid agents can be moved on the HIV-1 PR module, with extensions to the freed terminus, to improve the potency against HIV-1 PR.

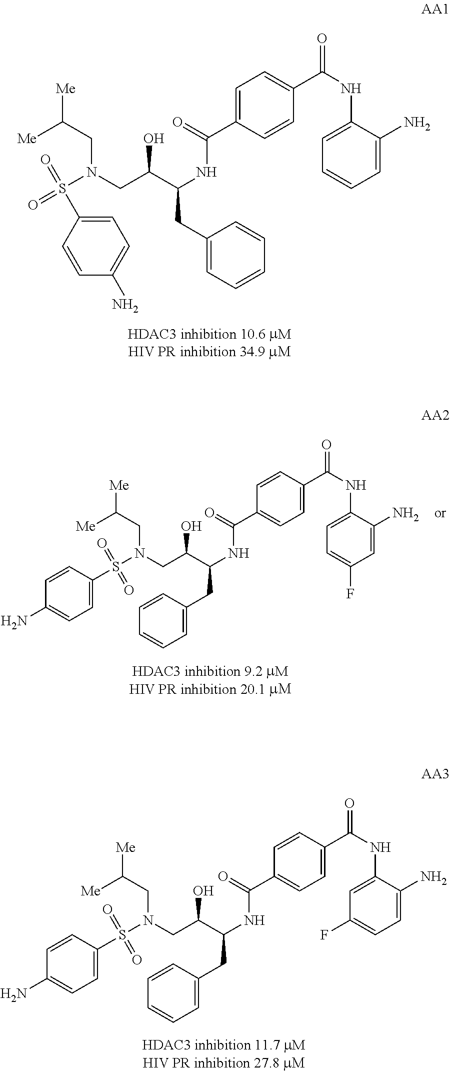 [SMILES: CC(C)CN(C[C@@H](O)[C@H](Cc1ccccc1)NC(=O)c1ccc(C(=O)Nc2ccccc2N)cc1)S(=O)(=O)c1ccc(N)cc1 ; CC(C)CN(C[C@@H](O)[C@H](Cc1ccccc1)NC(=O)c1ccc(C(=O)Nc2ccc(F)cc2N)cc1)S(=O)(=O)c1ccc(N)cc1 ; CC(C)CN(C[C@@H](O)[C@H](Cc1ccccc1)NC(=O)c1ccc(C(=O)Nc2cc(F)ccc2N)cc1)S(=O)(=O)c1ccc(N)cc1]

### Enzymatic Assays:

Histone deactylase 3 (HDAC3):
The HDAC3 assay kit measures HDAC activity using a fluorogenic substrate containing an acetylated lysine. In a 96-well plate, substrate (final concentration 20 mM) was mixed with bovine serum albumin (1 mg/mL) and assay buffer. A 10× solution of the test inhibitor was added, keeping a final 1% DMSO concentration. The reaction was initiated with the addition of HDAC3 enzyme (final concentration 2 ng/well), and the plate was incubated at 37° C. for 30 min. Controls consist of substrate control (no enzyme added), enzyme control (assay buffer with DMSO vehicle), and inhibitor control (final concentration of 2 mM trichostatin A). HDAC developer solution was then added to each well and incubated at room temperature for 15 min. Solution fluorescence was read at 360ex/450em nm on a plate reader. Assay was performed in triplicate, and IC₅₀ values were determined from a graph of percent inhibition as a function of concentration using GraphPad.

### HIV-1 Protease:

50 μL of a 200 nM solution of HIV-1 protease in buffer A (20 mM phosphate, 1 mM dithiothreitol, 1 mM EDTA, 20% glycerol, and 0.1% CHAPS at pH 5.5) was incubated with 10 μL of inhibitor solution in DMSO for 1 hour. This solution was then added to 40 μL of a 65 μM substrate solution in buffer A at 25° C. The change in fluorescence intensity at 430 nm (λ_{excitation} of 355 nm) upon addition of HIV-1 protease to the substrate solution was immediately measured in triplicate over a period of 3 hours. The final concentration of DMSO was maintained at 14%. The slope of the line obtained for each of the inhibitor concentrations was used to determine the percent inhibition using the following equation.

$$\% Inhibition = \frac{( slope of control ) - ( slope of PR - inhibitor solution )}{( slope of control )} \times 100$$

The percentage inhibition obtained in this manner was then plotted against the log[Inhibitor] and IC₅₀ values were determined (see Table 1) as the concentration of the inhibitor needed to reduce the activity of the HIV-1 protease by 50% using GraphPad Prism 10.

TABLE 1
Measured IC₅₀ values

| | | |
|---|---|---|
| Compound | HDAC3 | HIV-1 PR |

| | | | | |
|---|---|---|---|---|
| AA1 | 10.6 | μM | 34.9 | μM |
| AA2 | 9.2 | μM | 20.1 | μM |
| AA3 | 11.5 | μM | 27.8 | μM |
| NB-1 | 5.4 | μM | 925 | nM |
| NB-3 | 19 | nM | 104 | nM |
| NB-5 | 4.5 | μM | 45 | nM |

### Latently HIV-1-Infected Cells and Provirus Activation:

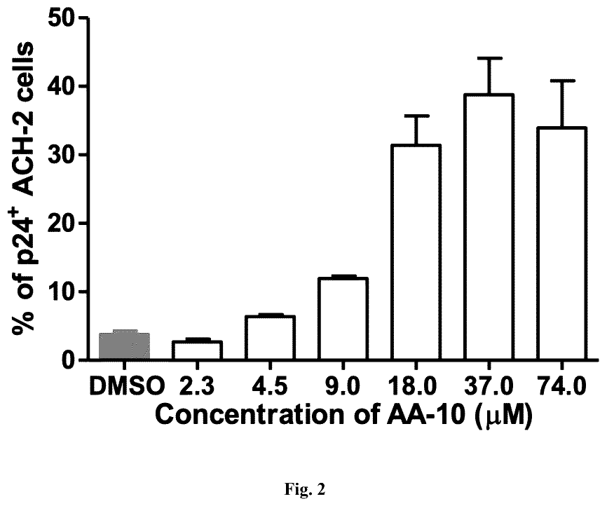

ACH-2 cells, a human T cell line latently infected with HIV-1LAV strain, was used to test the effects of compound AA2 on provirus activation. Cells were treated with varying concentrations of compound AA2 and DMSO as a negative control. Stimulated ACH-2 cells were subjected to intracellular staining of HIV-24 to determine provirus activation. These data demonstrate that compound AA2 robustly activated HIV-1 in latently infected cells in a concentration-dependent manner (FIG. 2).

### Inhibition of Cell-to-Cell Transmission of HIV-1:

Provirus-activated ACH-2 cells were used as HIV-1 donor cells, while A3.01 cells were used as target cells, as reported in J Med Virol. 2022, 94, 5434-5450. The ACH-2 HIV-1 donor cells were labeled with CellTrace Violet. After washing, either the labeled ACH HIV-1 donor cells alone, or cocultured with A3.01 cells at ratios (donor cells to target cells) of 5:1, were treated with various concentrations of the designed agents, with rhomadepsin (6 nM) with and without amprenavir (5 μM) as a positive control, or DMSO as a negative control. Cells were collected after 24 hours and stained with a viability dye, followed by p24 antibody treatment. Live cells were analyzed for p24 expression in A3.01 cells and ACH2 cells.

TABLE 2
In vitro and in cyto activities

| | | | Concentration | |
|---|---|---|---|---|
| | | | required for | |
| | | | maximal Latency | % inhibition of |
| | HDAC3 | HIV-1 PR | Reversal in T | cell transmission |
| Compound | IC₅₀ | IC₅₀ | cells | of HIV |
| NB-3 | 20 nM | 135 nM | 2.0 μM | 83% (at 10 μM) |
| NB-7 | 640 nM | 95 nM | 10 μM | 93% (at 40 μM) |
| NB-10 | 115 nM | 265 nM | 4.6 μM | 87% (at 20 μM) |
| NB-11 | 505 nM | 85 nM | 20 μM | 99% (at 40 μM) |

### EXPERIMENTAL

Solvents and reagents were obtained from commercial sources and utilized without further purification. ¹H and ¹³C NMR spectra presented were obtained in methanol-d₄ or CDCl₃ using a Bruker AV500 (500 MHz) or AV800 (800 MHz) spectrometer with tetramethylsilane as an internal standard. ¹H NMR data were reported as shown: chemical shift (S ppm) (multiplicity, coupling constant (Hz), integration). Chemical shifts were reported in downfield order in parts per million (δ ppm). Multiplicities are reported as follows: s=singlet, brs=broad singlet, d=doublet, t=triplet, q=quartet, m=multiplet, or combinations thereof. All the synthesized compounds were characterized using ¹H, and ¹³C NMR.

### Scheme 1—General Procedure

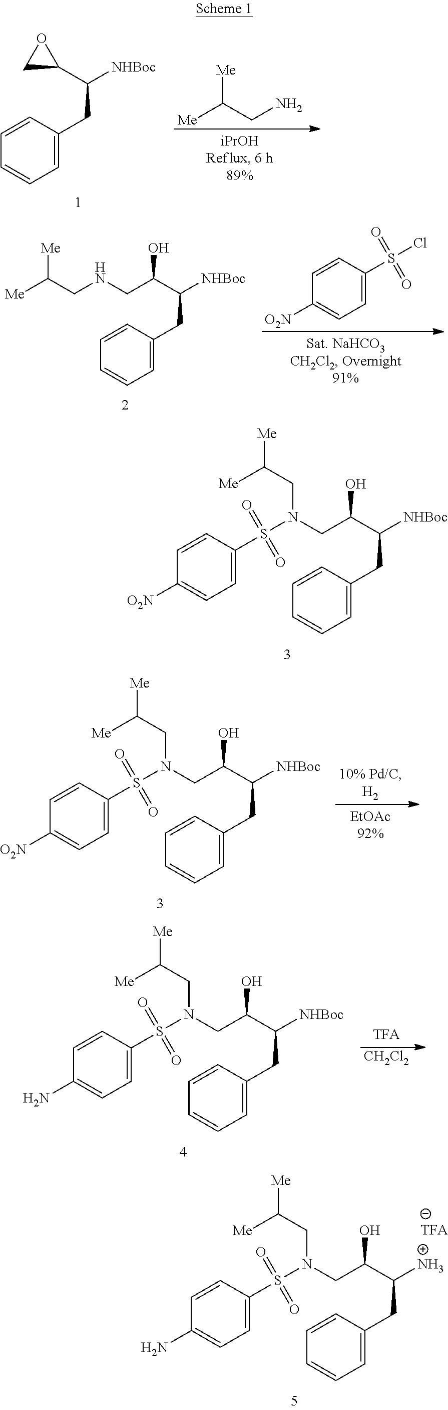

### Example 1

### tert-butyl((2S,3R)-3-hydroxy-4-(isobutylamino)-1-phenylbutan-2-yl)carbamate (2)

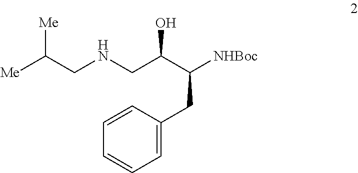 [SMILES: CC(C)CNC[C@@H](O)[C@H](Cc1ccccc1)NC(=O)OC(C)(C)C]

To a stirred solution of tert-butyl ((S)-1-((S)-oxiran-2-yl)-2-phenylethyl)carbamate (512 mg, 1.9 mmol, 1.0 eq) in isopropanol (16 ml, 0.12 M) at room temperature was added isobutylamine (1.16 ml, 11.6 mmol, 6.0 eq). This reaction mixture was refluxed for 6 h, after which the reaction mixture was concentrated under reduced pressure and the crude reaction mixture was purified by column chromatography on silica gel (5% MeOH in CH₂Cl₂) to obtain the secondary amine 2 (582 mg, 89%) as a white amorphous solid: ¹H NMR (400 MHz, MeOD) δ 7.25 (d, J=4.9 Hz, 4H), 7.20-7.12 (m, 1H), 3.81-3.70 (m, 2H), 2.92-2.71 (m, 2H), 2.61 (dq, J=7.5, 4.1 Hz, 2H), 2.38 (dd, J=6.9, 3.0 Hz, 2H), 1.76 (hept, J=6.7 Hz, 1H), 1.35 (s, 9H), 0.91 (d, J=6.6 Hz, 6H); ¹³C NMR (101 MHz, MeOD) δ 156.76, 138.65, 128.88, 127.81, 125.73, 78.63, 69.53, 57.01, 54.23, 51.88, 37.34, 27.42, 27.19, 19.41.

### Example 2

### tert-butyl((2S,3R)-3-hydroxy-4-((N-isobutyl-4-nitrophenyl)sulfonamido)-1-phenylbutan-2-yl)carbamate (3)

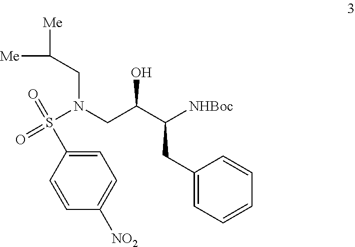 [SMILES: CC(C)CN(C[C@@H](O)[C@H](Cc1ccccc1)NC(=O)OC(C)(C)C)S(=O)(=O)c1ccc([N+](=O)[O-])cc1]

To a CH₂Cl₂ (34 ml, 0.05 M) solution of amine 2 (573 mg, 1.70 mmol, 1.0 eq) was added a saturated aqueous solution of sodium bicarbonate (34 ml, 0.050 M) followed by 4-nitrobenzenesulfonyl chloride (222 mg, 2.50 mmol, 1.50 eq) and the resulting mixture was stirred overnight at room temperature. The mixture was extracted with CH₂Cl₂ and dried over anhydrous MgSO₄, followed by the removal of the solvent under reduced pressure and purified by column chromatography on silica gel (1% MeOH in CH₂Cl₂) to obtain the sulfonamide 3 (810 mg, 91%) as a white amorphous solid: ¹H NMR (400 MHz, CDCl₃) δ 8.32 (d, J=8.4 Hz, 2H), 7.92 (d, J=8.4 Hz, 2H), 7.29 (t, J=7.5 Hz, 2H), 7.21 (d, J=7.6 Hz, 3H), 4.91 (d, J=9.2 Hz, 1H), 3.74 (d, J=8.8 Hz, 1H), 3.65 (d, J=8.2 Hz, 1H), 3.30 (dd, J=15.3, 9.1 Hz, 2H), 2.99 (dd, J=13.4, 8.6 Hz, 1H), 2.95 (s, 1H), 2.91 (s, 1H), 2.81 (dd, J=13.5, 6.6 Hz, 1H), 1.62 (p, J=7.0 Hz, 1H), 1.40 (s, 9H), 0.80 (t, J=5.7 Hz, 6H).

### Example 3

### tert-butyl((2S,3R)-4-((4-amino-N-isobutylphenyl)sulfonamido)-3-hydroxy-1-phenylbutan-2-yl)carbamate (4)

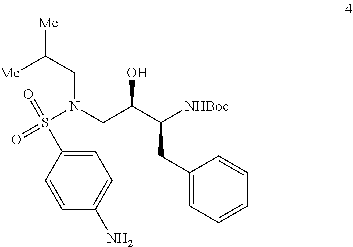 [SMILES: CC(C)CN(C[C@@H](O)[C@H](Cc1ccccc1)NC(=O)OC(C)(C)C)S(=O)(=O)c1ccc(N)cc1]

To a solution of sulfonamide 3 (810 mg, 1.20 mmol, 1.0 eq) in EtOAc (155 ml) was added 10% Pd/C (89 mg). The mixture was stirred at room temperature under H₂-filled balloon overnight. The reaction mixture was filtered over celite, and the filter cake was washed with EtOAc. Followed by removal of the solvent under reduced pressure, the reaction mixture was purified by column chromatography on silica gel (2% MeOH in CH₂Cl₂) to obtain aniline 4 (702 mg, 92%) as a white amorphous solid: ¹H NMR (400 MHz, CDCl₃) δ 7.43 (d, J=8.3 Hz, 2H), 7.20 (dp, J=15.3, 7.0 Hz, 5H), 6.58 (d, J=8.4 Hz, 2H), 5.07 (d, J=9.5 Hz, 1H), 4.32 (s, 2H), 3.68 (dd, J=23.0, 8.4 Hz, 3H), 3.20 (dd, J=15.2, 9.2 Hz, 1H), 2.93-2.80 (m, 3H), 2.72 (dd, J=15.2, 2.9 Hz, 1H), 2.59 (dd, J=13.3, 6.4 Hz, 1H), 1.60-1.49 (m, 1H), 1.36 (s, 9H), 0.75 (dd, J=9.4, 6.5 Hz, 6H); ¹³C NMR (101 MHz, CDCl₃) δ 155.97, 151.00, 138.09, 129.20, 128.29, 126.24, 125.51, 113.87, 79.29, 69.64, 58.67, 54.28, 54.08, 38.69, 30.82, 28.23, 27.16, 19.98, 19.62.

### Example 4

### General Procedure Described for Boc Deprotection of Compound (4)

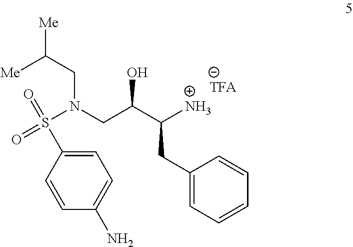 [SMILES: CC(C)CN(C[C@@H](O)[C@@H]([NH3+])Cc1ccccc1)S(=O)(=O)c1ccc(N)cc1]

Stirring sulfonamide 4 in an 0.5 M solution of trifluoroacetic acid in CH₂Cl₂ for 1 h afforded Boc deprotection. The solvent was evaporated under reduced pressure and the crude compound 5 was carried forward without purification.

### Scheme 2: Synthetic Scheme Towards the Synthesis of Compound AA1

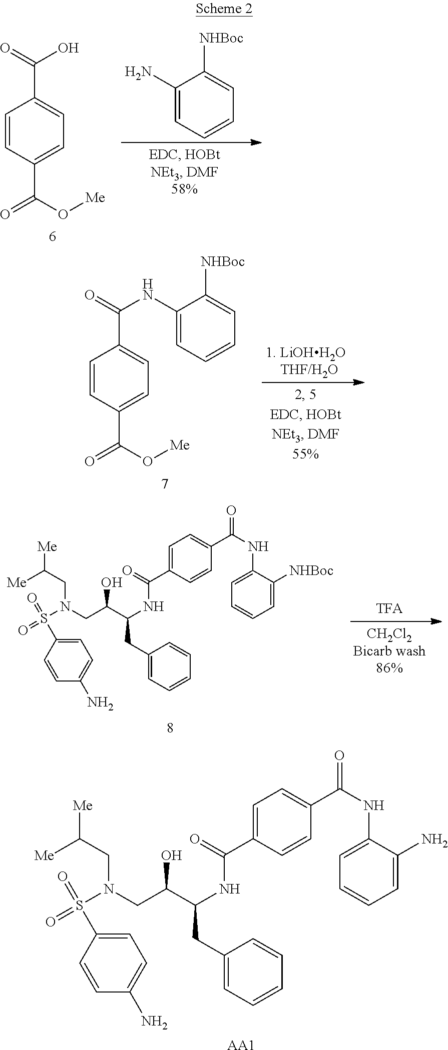

### Example 5

### Methyl 4-((2-((tert-butoxycarbonyl)amino)phenyl)carbamoyl)benzoate (7)

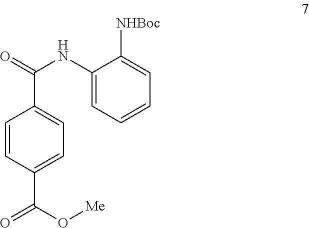 [SMILES: COC(=O)c1ccc(C(=O)Nc2ccccc2NC(=O)OC(C)(C)C)cc1]

A solution of benzoic acid 6 (353 mg, 1.96 mmol, 1.0 eq) in DMF (0.10 M, 19.6 ml) was added EDC (753 mg, 3.90 mmol, 2.0 eq), HOBt (331 mg, 2.16 mmol, 1.10 eq), and triethylamine (1.4 ml, 9.8 mmol, 5.0 eq) was stirred for 5 minutes at room temperature and before tert-butyl (2-aminophenyl)carbamate was added to it and stirred overnight. The reaction mixture was quenched with 1 M HCl and the organics extracted with EtOAc thrice. The combined organic layer was washed with saturated sodium bicarbonate followed by brine and dried over anhydrous MgSO₄. The solvent was evaporated under reduced pressure and purified by column chromatography on silica gel (30% EtOAc in Hexanes) to obtain compound 7 (421 mg, 58%) as a yellowish-white solid.

### Example 6

### tert-butyl(2-(4-(((2S,3R)-4-((4-amino-N-isobutylphenyl)sulfonamido)-3-hydroxy-1-phenylbutan-2- yl)carbamoyl)benzamido)phenyl)carbamate (8)

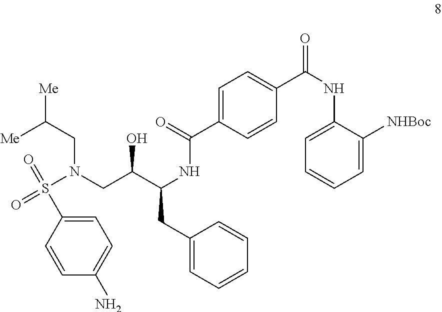 [SMILES: CC(C)CN(C[C@@H](O)[C@H](Cc1ccccc1)NC(=O)c1ccc(C(=O)Nc2ccccc2NC(=O)OC(C)(C)C)cc1)S(=O)(=O)c1ccc(N)cc1]

To a solution of 7 (28.9 mg, 0.078 mmol, 1 eq) in 50% THF/H₂O (0.78 ml, 0.1 M) was added LiOH·H₂O (5.0 mg, 0.12 mmol, 1.5 eq) and stirred for an hour. The reaction was quenched with 1 M HCl and extracted with EtOAc thrice. The combined organic layer was washed with brine and dried over anhydrous MgSO₄. The solvent was evaporated under reduced pressure and the crude colorless solid was dissolved in DMF (0.78 ml, 0.1 M) to which was added EDC (29.9 mg, 0.156 mmol, 2.0 eq), HOBt (13 mg, 0.086 mmol, 1.1 eq) and triethylamine (28 μl, 0.19 mmol, 2.5 eq) and stirred for 5 minutes. A DMF solution of triethylamine (28 μl, 0.19 mmol, 2.5 eq) and crude 5 was added and stirred overnight. The reaction was quenched with 1 M HCl and extracted with EtOAc thrice. The combined organic layer was washed with saturated sodium bicarbonate and brine and dried over anhydrous MgSO₄. The solvent was evaporated under reduced pressure and purified using column chromatography on silica gel (2% MeOH in CH₂Cl₂) to obtain compound 8 as a yellow solid (31 mg, 55%): ¹H NMR (400 MHz, CDCl₃) δ 9.51 (s, 1H), 7.94 (d, J=8.1 Hz, 2H), 7.79-7.68 (m, 3H), 7.39-7.33 (m, 2H), 7.28 (d, J=1.0 Hz, 1H), 7.26-7.10 (m, 6H), 6.92 (d, J=9.0 Hz, 1H), 6.56-6.48 (m, 2H), 4.29-4.18 (m, 3H), 3.87 (d, J=8.6 Hz, 1H), 3.18-3.02 (m, 3H), 2.97-2.79 (m, 2H), 2.73-2.60 (m, 1H), 1.65-1.52 (m, 1H), 1.49 (s, 9H), 0.79 (dd, J=11.4, 6.6 Hz, 6H); ¹³C NMR (101 MHz, CDCl₃) δ 167.06, 164.81, 154.64, 150.86, 137.79, 137.05, 136.95, 130.42, 130.00, 129.22, 129.19, 128.45, 127.63, 127.11, 126.52, 126.07, 125.77, 125.61, 125.34, 124.42, 113.92, 81.43, 69.91, 58.90, 54.21, 53.81, 38.02, 29.59, 28.20, 27.33, 19.96, 19.66.

### Example 7

### N¹-((2S,3R)-4-((4-amino-N-isobutylphenyl)sulfonamido)-3-hydroxy-1-phenylbutan-2-yl)-N⁴-(2-aminophenyl)terephthalamide (AA1)

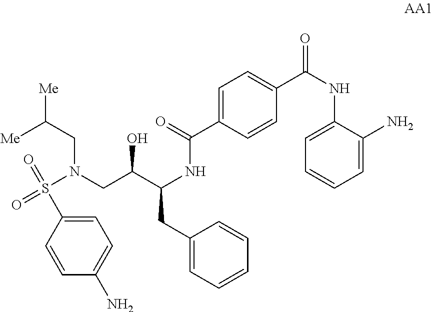 [SMILES: CC(C)CN(C[C@@H](O)[C@H](Cc1ccccc1)NC(=O)c1ccc(C(=O)Nc2ccccc2N)cc1)S(=O)(=O)c1ccc(N)cc1]

The general procedure described for Boc deprotection of compound 4 was followed for compound 8 and was followed by dissolving the crude reaction mixture in EtOAc and, washing it with saturated sodium bicarbonate thrice and drying the EtOAc layer in anhydrous MgSO₄. The solvent was evaporated under reduced pressure to obtain compound AA1 as a brownish-white solid (18 mg, 86%).

### Scheme 3: Synthetic Scheme Towards the Synthesis of Compound AA2

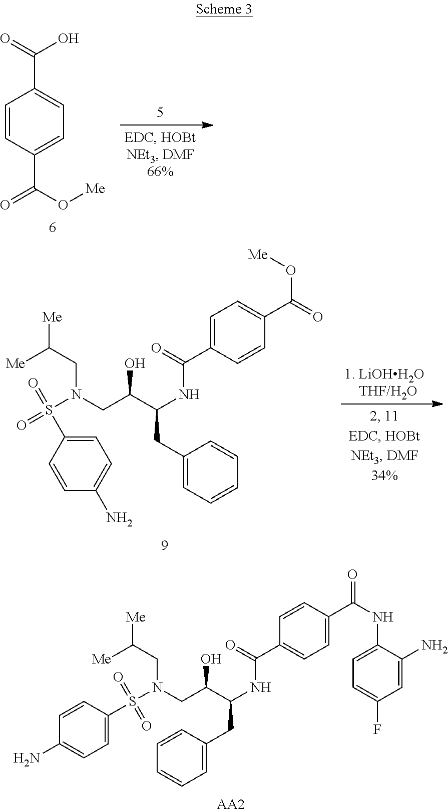

### Example 8

### Methyl4-(((2S,3R)-4-((4-amino-N-isobutylphenyl)sulfonamido)-3-hydroxy-1-phenylbutan-2-yl)carbamoyl)benzoate (9)

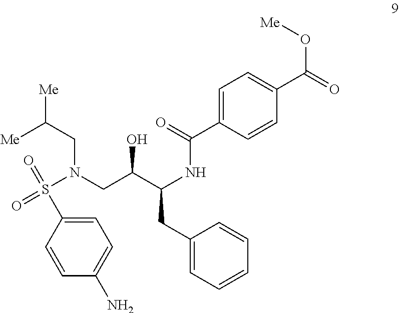 [SMILES: COC(=O)c1ccc(C(=O)N[C@@H](Cc2ccccc2)[C@H](O)CN(CC(C)C)S(=O)(=O)c2ccc(N)cc2)cc1]

To a solution of benzoic acid 6 (18 mg, 0.098 mmol, 1.0 eq) in DMF (0.1 M, 0.98 ml) was added EDC (38 mg, 0.20 mmol, 2.0 eq), HOBt (17 mg, 0.11 mmol, 1.1 eq), and triethylamine (28 μl, 0.19 mmol, 2.5 eq) and the reaction was stirred for 5 minutes. A DMF solution of triethylamine (28 μl, 0.19 mmol, 2.5 eq) was added to crude 5 and the reaction stirred overnight. The reaction was quenched with 1 M HCl and extracted with EtOAc thrice. The combined organic layer was washed with saturated sodium bicarbonate and brine and dried over anhydrous MgSO₄. The solvent was evaporated under reduced pressure and purified using column chromatography on silica gel (1% MeOH in CH₂Cl₂) to obtain compound 9 as a yellow solid (36 mg, 66%): ¹H NMR (400 MHz, CDCl₃) δ 8.12-8.03 (m, 2H), 7.80-7.73 (m, 2H), 7.43-7.34 (m, 2H), 7.33-7.24 (m, 4H), 7.24-7.16 (m, 1H), 6.82 (d, J=9.0 Hz, 1H), 6.58-6.50 (m, 2H), 4.23 (q, J=8.1 Hz, 2H), 3.94 (s, 3H), 3.87 (ddd, J=8.9, 3.9, 1.4 Hz, 1H), 3.10 (hept, J=8.7 Hz, 3H), 2.96-2.80 (m, 2H), 2.67 (dd, J=13.3, 6.5 Hz, 1H), 1.58 (ddq, J=13.0, 8.2, 6.5 Hz, 1H), 0.79 (dd, J=9.5, 6.6 Hz, 6H); ¹³C NMR (101 MHz, CDCl₃) δ 166.96, 166.17, 150.70, 138.11, 137.73, 132.76, 129.79, 129.20, 128.46, 126.90, 126.52, 125.52, 113.95, 69.85, 59.00, 54.34, 53.74, 52.34, 38.08, 29.59, 27.34, 19.96, 19.64.

### Example 9

N¹-(2-amino-4-fluorophenyl)-N⁴-((2S,3R)-4-((4-amino-N-isobutylphenyl)sulfonamido)-3-hydroxy-1-phenylbutan-2-yl)terephthalamide (AA2)

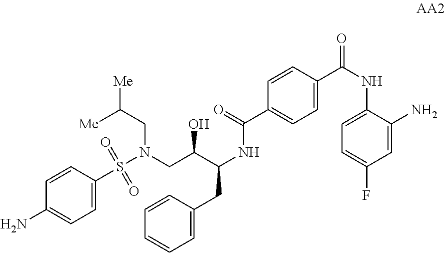 [SMILES: CC(C)CN(C[C@@H](O)[C@H](Cc1ccccc1)NC(=O)c1ccc(C(=O)Nc2ccc(F)cc2N)cc1)S(=O)(=O)c1ccc(N)cc1]

To a 50% THE/H₂O (0.33 ml, 0.1 M) solution of compound 9 (18 mg, 0.033 mmol, 1.0 eq) was added LiOH·H₂O (2.1 mg, 0.049 mmol, 1.5 eq) and stirred for an hour. The reaction was quenched with 1 M HCl and extracted with EtOAc thrice. The combined organic layer was washed with brine and dried over anhydrous MgSO4. The solvent was evaporated under reduced pressure and the crude yellow solid was dissolved in DMF (0.33 ml, 0.10 M) and was added EDC (13 mg, 0.066 mmol, 2.0 eq), HOBt (6 mg, 0.04 mmol, 1.1 eq) and triethylamine (12 μl, 0.90 mmol, 2.5 eq) and stirred for 5 minutes. A DMF solution of triethylamine (12 μl, 0.9 mmol, 2.5 eq) and crude 5 was added and stirred overnight. The reaction was quenched with 1 M HCl and extracted with EtOAc thrice. The combined organic layer was washed with saturated sodium bicarbonate and brine and dried over anhydrous MgSO₄. The solvent was evaporated under reduced pressure and purified using column chromatography on silica gel (2% MeOH in CH₂Cl₂) to obtain compound AA2 as a brown solid (7.3 mg, 34%).

### Scheme 4: Synthetic Scheme Towards the Synthesis of Compound AA3

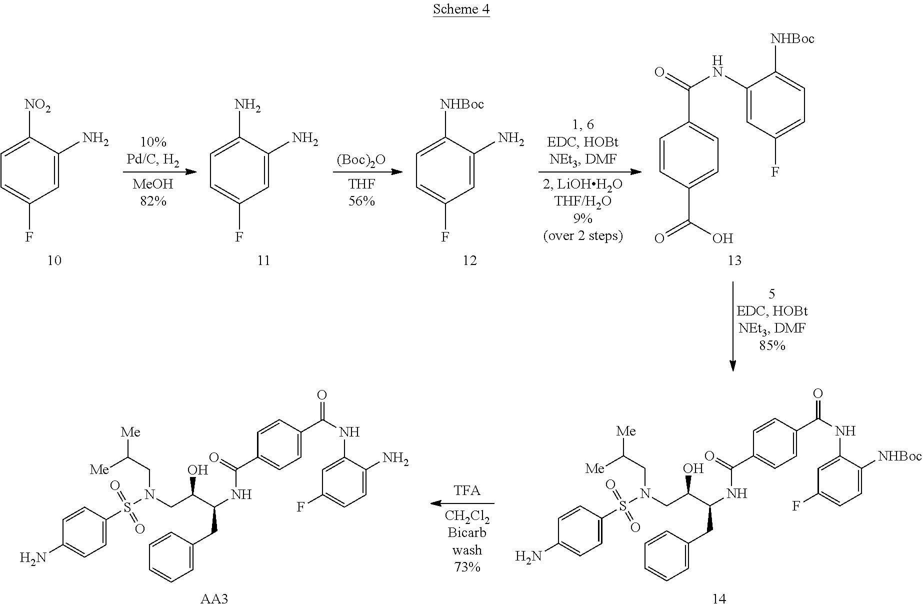

### Example 10

### 4-fluorobenzene-1,2-diamine (11)

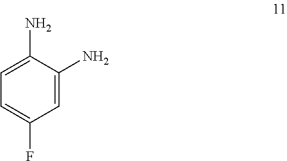

To a MeOH (32 ml, 0.1 ml) solution of compound 10 (503 mg, 3.20 mmol) was added 10% Pd/C (55 mg), which was placed under an H₂ atmosphere at balloon pressure and stirred overnight. The reaction mixture was filtered over Celite, and the filter cake was washed with MeOH. Following the removal of the solvent under reduced pressure, the reaction mixture was purified by column chromatography on silica gel (1% MeOH in CH₂Cl₂) to obtain diamine 11 (331 mg, 82%) as a dark brown solid: ¹H NMR (400 MHz, CDCl₃) δ 6.61 (dd, J=8.4, 5.5 Hz, 1H), 6.49-6.33 (m, 2H), 3.39 (s, 4H); ¹³C NMR (101 MHz, CDCl₃) δ 158.88, 156.53, 136.70, 129.65, 117.64, 117.55, 105.32, 105.10, 103.23, 102.98.

### Example 12

### tert-butyl (2-amino-4-fluorophenyl)carbamate (12)

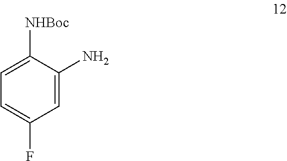 [SMILES: CC(C)(C)OC(=O)Nc1ccc(F)cc1N]

To a THF (0.98 ml, 0.50 M) solution of diamine 11 (62 mg, 0.49 mmol) was added di-tert-butyl dicarbonate (108 mg, 0.490 mmol) at room temperature and stirred overnight. The reaction mixture was concentrated under reduced pressure and purified using column chromatography on silica gel to obtain compound 12 (62 mg, 56%) as a brownish white solid: ¹H NMR (400 MHz, CDCl₃) δ 7.08 (dd, J=9.0, 5.8 Hz, 1H), 6.47-6.37 (m, 2H), 6.14 (s, 1H), 3.89 (s, 2H), 1.49 (s, 9H); ¹³C NMR (101 MHz, CDCl₃) δ 162.54, 160.13, 154.16, 142.83, 142.72, 127.17, 119.68, 105.42, 105.20, 103.32, 80.58, 28.20.

### Example 13

### tert-butyl (2-(4-(((2S,3R)-4-((4-amino-N-isobutylphenyl)sulfonamido)-3-hydroxy-1-phenylbutan-2- yl)carbamoyl)benzamido)-4-fluorophenyl)carbamate (14)

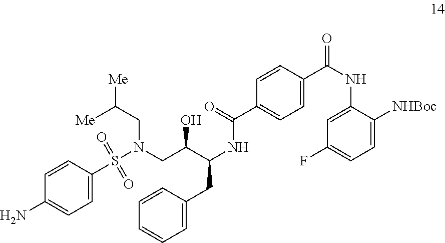 [SMILES: CC(C)CN(C[C@@H](O)[C@H](Cc1ccccc1)NC(=O)c1ccc(C(=O)Nc2cc(F)ccc2NC(=O)OC(C)(C)C)cc1)S(=O)(=O)c1ccc(N)cc1]

To a solution of benzoic acid 6 (40 mg, 0.22 mmol, 1.0 eq) in DMF (0.1 M, 2.2 ml) was added EDC (85 mg, 0.45 mmol, 2.0 eq), HOBt (38 mg, 0.25 mmol, 1.1 eq), and triethylamine (155 μl, 1.11 mmol, 5 eq) which was stirred for 5 minutes at room temperature and before amine 12 was added to it and further stirred overnight. The reaction mixture was quenched with 1 M HCl and extracted the organics with EtOAc thrice. The combined organic layer was washed with saturated sodium bicarbonate and brine and dried over anhydrous MgSO₄. The solvent was evaporated under reduced pressure and purified by column chromatography on silica gel (30% EtOAc in Hexanes) to obtain an inseparable mixture of compound 13 and amine 12 (17 mg, 20%) as a brown oil. This mixture was dissolved in a 50% THF/H₂O (0.33 ml, 0.1 M) solution and was added LiOH·H₂O (2.8 mg, 0.066 mmol, 1.5 eq) and stirred for an hour. The reaction was quenched with 1 M HCl and extracted with EtOAc thrice. The combined organic layer was treated with saturated sodium bicarbonate thrice to take the acid 13 to the aqueous layer while leaving behind the organic impurities. This aqueous layer was acidified using 1 M HCl and then extracted with EtOAc thrice and washed with brine and dried over anhydrous MgSO₄. The solvent was evaporated under reduced pressure and the crude yellow solid (13, 7 mg, 0.02 mmol) was obtained.
This crude acid was dissolved in DMF (0.195 ml, 0.1 M) to which was added EDC (7.5 mg, 0.039 mmol, 2 eq), HOBt (3.3 mg, 0.022 mmol, 1.1 eq) and triethylamine (7 μl, 0.049 mmol, 2.5 eq) and stirred for 5 minutes. A DMF solution of triethylamine (7 μl, 0.049 mmol, 2.5 eq) and crude 5 was added and stirred overnight. The reaction was quenched with 1 M HCl and extracted with EtOAc thrice. The combined organic layer was washed with saturated sodium bicarbonate and brine and dried over anhydrous MgSO₄. The solvent was evaporated under reduced pressure and purified using column chromatography on silica gel (2% MeOH in CH₂Cl₂) to obtain compound 14 as a yellow solid (12 mg, 85%): ¹H NMR (400 MHz, CDCl₃) δ 9.54 (s, 1H), 7.98 (q, J=8.6 Hz, 3H), 7.77 (dd, J=9.4, 5.4 Hz, 3H), 7.39 (d, J=8.2 Hz, 2H), 7.34-7.18 (m, 6H), 7.13 (dd, J=8.9, 5.5 Hz, 1H), 6.90-6.81 (m, 2H), 6.79 (s, 1H), 6.55 (d, J=8.3 Hz, 2H), 4.23 (q, J=8.1 Hz, 2H), 3.92-3.84 (m, 1H), 3.19-3.02 (m, 3H), 2.97-2.81 (m, 7H), 2.69 (dd, J=13.2, 6.5 Hz, 1H), 1.59 (dt, J=14.2, 6.5 Hz, 1H), 1.52 (s, 9H), 1.10 (s, 1H), 0.80 (dd, J=10.0, 6.5 Hz, 6H); ¹³C NMR (101 MHz, CDCl₃) δ 166.89, 164.41, 159.18, 154.86, 137.74, 137.23, 136.82, 129.22, 128.47, 127.60, 127.19, 126.54, 124.88, 113.98, 81.90, 69.87, 59.02, 54.32, 53.83, 38.04, 29.60, 28.18, 27.36, 19.95, 19.65.

### Example 14

### N¹-(2-amino-5-fluorophenyl)-N⁴-((2S,3R)-4-((4-amino-N-isobutylphenyl)sulfonamido)-3-hydroxy-1-phenylbutan-2-yl)terephthalamide (AA3)

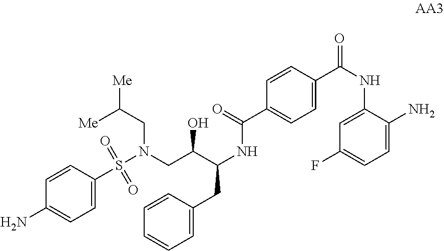 [SMILES: CC(C)CN(C[C@@H](O)[C@H](Cc1ccccc1)NC(=O)c1ccc(C(=O)Nc2cc(F)ccc2N)cc1)S(=O)(=O)c1ccc(N)cc1]

The general procedure described for Boc deprotection of compound 4 was followed for compound 14 and was followed by dissolving the crude reaction mixture in EtOAc and washing it with saturated sodium bicarbonate thrice and drying the EtOAc layer in anhydrous MgSO₄. The solvent was evaporated under reduced pressure to obtain compound AA3 as a yellowish-white solid (7 mg, 73%): ¹H NMR (400 MHz, CDCl₃) δ 8.53 (s, 1H), 7.84 (d, J=8.0 Hz, 2H), 7.67 (d, J=8.0 Hz, 2H), 7.47 (d, J=9.7 Hz, 1H), 7.40 (d, J=8.4 Hz, 2H), 7.24 (dq, J=20.0, 7.2 Hz, 7H), 6.93 (d, J=9.1 Hz, 1H), 6.80 (t, J=5.7 Hz, 2H), 6.57 (d, J=8.6 Hz, 2H), 4.22 (q, J=8.2 Hz, 2H), 3.89-3.81 (m, 1H), 3.19-3.00 (m, J=8.6, 8.2 Hz, 3H), 2-99-2.90 (m, 1H), 2.90-2.78 (m, 1H), 2.67 (dd, J=13.2, 6.3 Hz, 1H), 1.55 (p, J=6.7 Hz, 1H), 0.79 (dd, J=13.8, 6.6 Hz, 6H); ¹³C NMR (101 MHz, CDCl₃) δ 166.86, 164.79, 150.78, 137.67, 137.06, 136.78, 129.19, 128.48, 127.53, 127.24, 126.56, 125.52, 114.03, 69.89, 60.31, 58.95, 54.29, 53.80, 38.09, 29.60, 27.36, 19.92, 19.61.

### Scheme 5: Synthetic Scheme Towards the Synthesis of Compound AA12

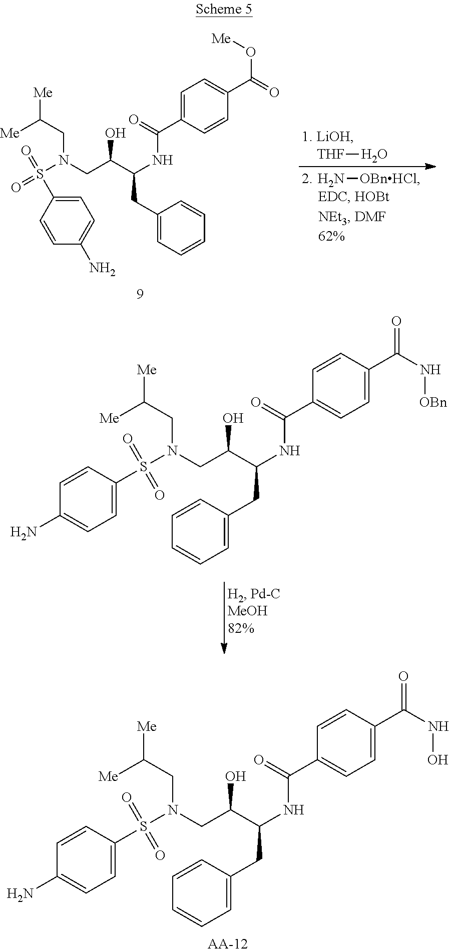

### Example 15

### N¹-((2S,3R)-4-((4-amino-N-isobutylphenyl)sulfonamido)-3-hydroxy-1-phenylbutan-2-yl)-N⁴-(benzyloxy)terephthalamide (15)

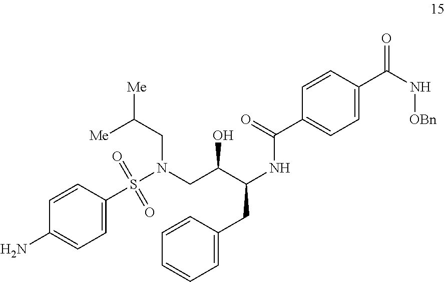 [SMILES: CC(C)CN(C[C@@H](O)[C@H](Cc1ccccc1)NC(=O)c1ccc(C(=O)NOCc2ccccc2)cc1)S(=O)(=O)c1ccc(N)cc1]

To a solution of compound 9 (1 eq) in THF/water (0.5 M) was added LiOH·H₂O (1.5 eq). The reaction was stirred for an hour, after which 1 M HCl was added to quench the reaction. EtOAc was used to extract the acid into the organic layer, which was subsequently washed with brine before drying over MgSO₄. The organic layer was concentrated under reduced pressure to yield the crude acid, which was redissolved in DMF (0.2 M, 0.23 ml). HATU (19 mg, 0.05 mmol, 1.1 eq), and diisopropylethylamine (24 μl, 0.14 mmol, 3 eq) were added to this solution and stirred for 5 minutes. A solution of triethylamine (14 μl, 0.09 mmol, 1.9 eq) and O-benzylhydroxylamine hydrochloride in DMF were added to this mixture and stirred overnight. The reaction was quenched with 1 M HCl and extracted with EtOAc thrice. The combined organic layer was washed with saturated sodium bicarbonate and brine and dried over anhydrous MgSO₄. The solvent was evaporated under reduced pressure and purified using column chromatography on silica gel (2% MeOH in CH₂Cl₂) to obtain compound 15 as a brown solid (19 mg, 62%): ¹H NMR (400 MHz, CDCl₃) δ 9.56 (s, 1H), 7.57 (s, 4H), 7.49-7.12 (m, 12H), 6.94 (d, J=9.1 Hz, 1H), 6.51 (d, J=8.2 Hz, 2H), 5.02 (s, 2H), 4.23 (q, J=8.2 Hz, 1H), 3.85 (dd, J=9.3, 3.5 Hz, 1H), 3.08 (tt, J=21.3, 11.6 Hz, 3H), 2.87 (ddd, J=31.1, 14.2, 5.9 Hz, 2H), 2.65 (dd, J=13.3, 6.3 Hz, 1H), 1.58 (s, 1H), 0.78 (dd, J=14.6, 6.5 Hz, 6H); ¹³C NMR (101 MHz, CDCl₃) δ 167.06, 150.70, 137.72, 135.09, 129.21, 129.14, 128.76, 128.54, 128.45, 127.32, 127.09, 126.53, 125.41, 114.10, 78.34, 69.85, 58.82, 54.12, 53.68, 38.04, 27.33, 19.96, 19.63; ¹³C NMR (126 MHz, CDCl₃) δ 167.18, 162.65, 150.89, 137.87, 135.23, 135.21, 129.34, 129.28, 128.85, 128.65, 128.57, 127.45, 127.21, 126.65, 125.53, 114.19, 78.43, 69.96, 58.95, 54.25, 53.82, 38.17, 27.45, 20.08, 19.76.

### Example 16

### N¹-((2S,3R)-4-((4-amino-N-isobutylphenyl)sulfonamido)-3-hydroxy-1-phenylbutan-2-yl)-N⁴-hydroxyterephthalamide (AA-12)

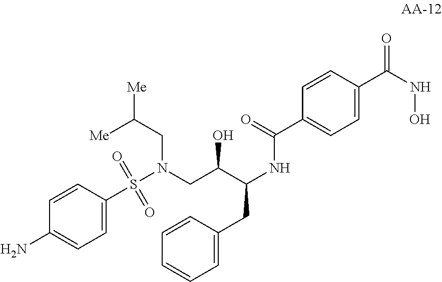 [SMILES: CC(C)CN(C[C@@H](O)[C@H](Cc1ccccc1)NC(=O)c1ccc(C(=O)NO)cc1)S(=O)(=O)c1ccc(N)cc1]

To a solution of compound 15 (16 mg, 0.025 mmol) in MeOH (0.25 ml, 0.1 M) was added 10% Pd/C (1.6 mg) and which was stirred overnight in a hydrogen atmosphere at balloon pressure. The reaction mixture was filtered over celite, and the filter cake was washed with MeOH. Solvent was removed from the filtrate under reduced pressure, the residue was purified by column chromatography on silica gel (2% MeOH in CH₂Cl₂) to obtain diamine AA-12 (11.2 mg, 82%) as a dark brown solid: ¹H NMR (500 MHz, MeOD) δ 7.95-7.88 (m, 2H), 7.81-7.75 (m, 2H), 7.43-7.36 (m, 2H), 7.32-7.22 (m, 4H), 7.21-7.14 (m, 1H), 6.66-6.60 (m, 2H), 4.56 (s, 1H), 4.32 (ddd, J=8.8, 6.8, 2.2 Hz, 1H), 3.95 (ddd, J=8.3, 4.3, 2.2 Hz, 1H), 3.22 (dd, J=14.9, 4.3 Hz, 1H), 3.07-2.91 (m, 3H), 2.84 (qd, J=13.6, 7.5 Hz, 2H), 1.77 (dp, J=14.0, 7.0 Hz, 1H), 0.80 (dd, J=8.0, 6.6 Hz, 6H); ¹³C NMR (126 MHz, MeOD) δ 170.07, 168.20, 152.93, 138.46, 137.50, 136.36, 129.04, 128.03, 127.42, 127.10, 126.05, 124.47, 113.05, 70.01, 57.67, 54.13, 52.70, 36.93, 26.87, 19.12, 19.05.

### Scheme 6: Synthetic Scheme Towards the Synthesis of Compound AA13

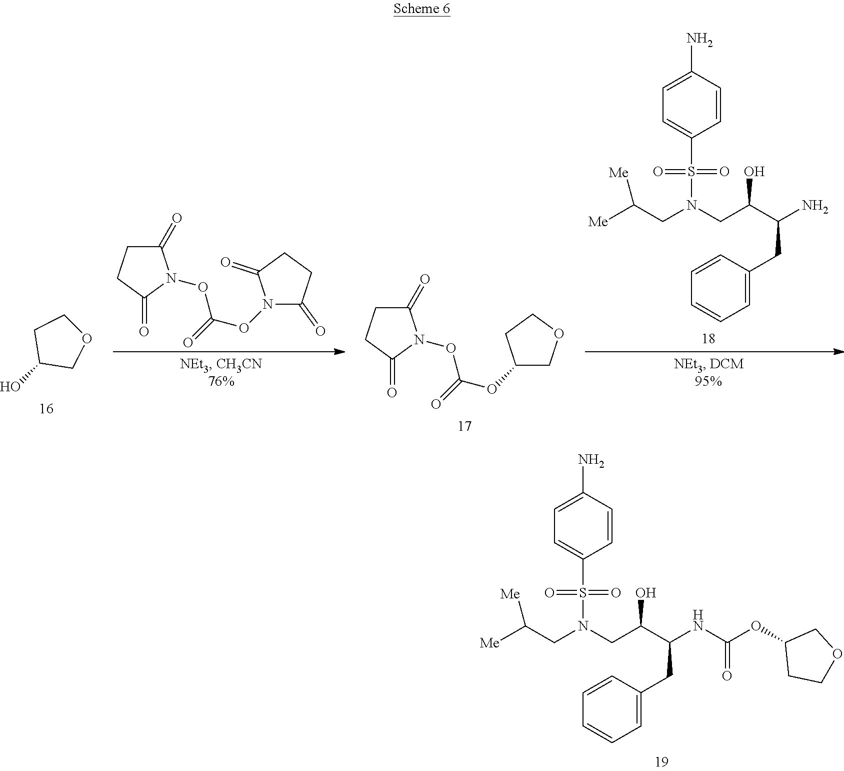

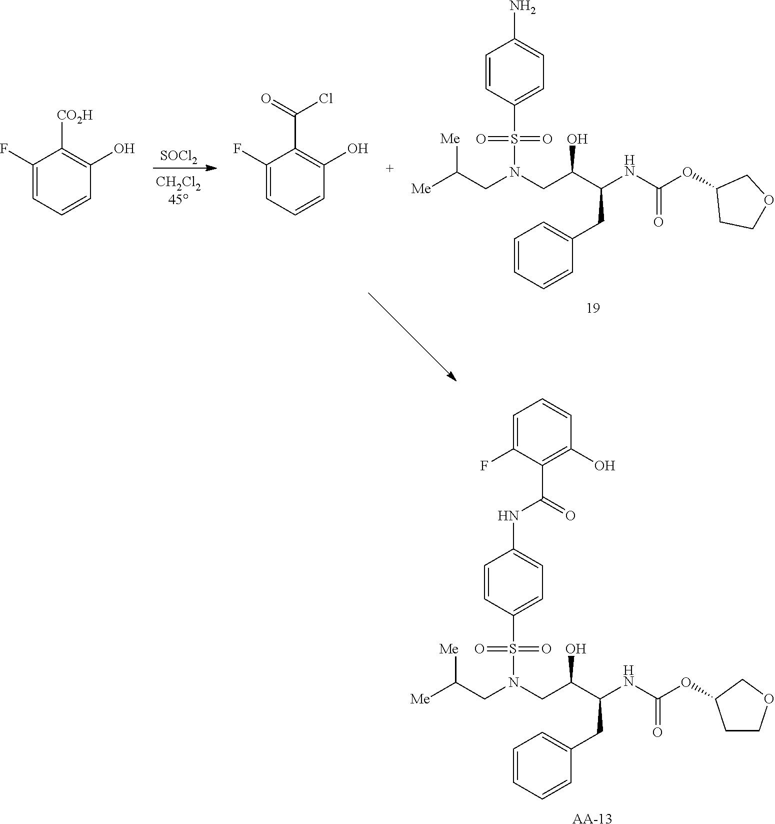

### Example 17

### (R)-2,5-Dioxopyrrolidin-1-yl (tetrahydrofuran-3-yl) carbonate (17)

To a solution of (R)-tetrahydrofuran-3-ol (16) (109.7 mg, 1.22 mmol) in acetonitrile (4.8 ml, 0.25 M) was added triethylamine (0.521 ml, 3.74 mmol) and bis(2,5-dioxopyrrolidin-1-yl) carbonate (478 mg, 1.90 mmol) and the reaction was stirred for 7 hours. The reaction mixture was concentrated, and the residue was treated with saturated aqueous sodium bicarbonate. The resulting mixture was extracted with EtOAc (3×), and the combined organic extracts were dried over MgSO₄. The solvent was subsequently evaporated under reduced pressure and the crude mixture was purified by column chromatography over silica gel (1% MeOH in CHCl₃ as the eluent) to yield the carbonate 17 (218 mg, 76%): ¹H NMR (400 MHz, CDCl₃) δ 5.31 (ddt, J=6.0, 4.1, 1.9 Hz, 1H), 3.97-3.80 (m, 4H), 2.79 (s, 4H), 2.26-2.10 (m, 2H); ¹³C NMR (101 MHz, CDCl₃) δ 168.59, 151.10, 82.26, 72.29, 66.72, 32.47, 25.34.

### Example 18

### (S)-Tetrahydrofuran-3-yl (2S,3R)-4-((4-amino-N-isobutylphenyl)sulfonamido)-3-hydroxy-1-phenylbutan-2-yl)carbamate (19)

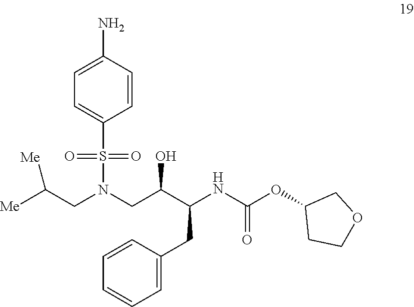 [SMILES: CC(C)CN(C[C@@H](O)[C@H](Cc1ccccc1)NC(=O)O[C@H]1CCOC1)S(=O)(=O)c1ccc(N)cc1]

To a solution of compound 18 (421 mg, 0.85 mmol) in CH₂Cl₂ (43 ml, 0.02 M) was added triethylamine (1.19 ml, 8.5 mmol) and compound 17 (218 mg, 0.95 mmol). This mixture was stirred for 3 hours. The reaction mixture was concentrated, and the crude product was purified by column chromatography over silica gel (2% MeOH in CH₂Cl₂) to provide compound 19 (410 mg, 95%) as an off-white solid: ¹H NMR (400 MHz, CDCl₃) δ 7.48-7.41 (m, 2H), 7.30-7.14 (m, 5H), 6.65-6.57 (m, 2H), 5.29 (d, J=9.5 Hz, 1H), 5.16-5.04 (m, 1H), 4.22 (s, 1H), 3.91-3.64 (m, 7H), 3.19 (dd, J=15.2, 9.2 Hz, 1H), 2.95-2.81 (m, 3H), 2.73 (dd, J=15.2, 2.8 Hz, 1H), 2.63 (dd, J=13.3, 6.4 Hz, 1H), 2.09 (qd, J=8.0, 3.9 Hz, 1H), 1.94 (dt, J=11.8, 5.0 Hz, 1H), 1.57-1.47 (m, 1H), 0.77 (q, J=6.9 Hz, 6H); ¹³C NMR (101 MHz, CDCl₃) δ 156.11, 150.90, 137.79, 129.26, 129.22, 128.36, 126.40, 125.65, 113.91, 75.19, 73.15, 69.58, 66.84, 58.70, 54.72, 54.33, 38.77, 32.68, 30.83, 27.18, 19.97, 19.62.

### Example 19

### (S)-Tetrahydrofuran-3-yl (2S,3R)-4-(((4-(2-fluoro-6-hydroxybenzamido)-N-isobutyl-phenyl)sulfonamido)-3-hydroxy-1-phenylbutan-2-yl)carbamate (AA-13)

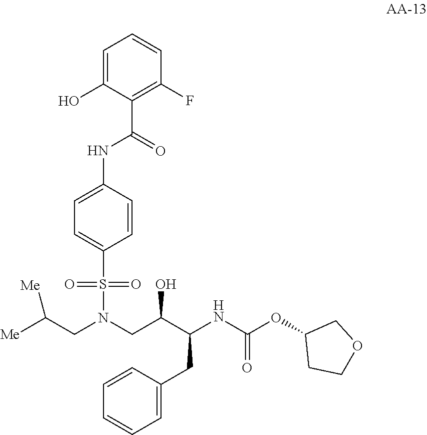 [SMILES: CC(C)CN(C[C@@H](O)[C@H](Cc1ccccc1)NC(=O)O[C@H]1CCOC1)S(=O)(=O)c1ccc(NC(=O)c2c(O)cccc2F)cc1]

To a solution of 2-fluoro-6-hydroxybenzoic acid (20) (17 mg, 0.11 mmol, 1 eq) in CH₂Cl₂ (0.15 M) was added thionyl chloride (0.016 ml, 0.22 mmol, 2 eq) which was refluxed for 3 hours. After 3 hours, the reaction was cooled down to room temperature and the solvent was evaporated off under reduced pressure. This crude acid chloride (21) was then added to a solution of compound 19 (32 mg, 0.061 mmol, 1 eq) in CH₂Cl₂ (0.15 M) and stirred overnight. The solvent was evaporated off under reduced pressure and the crude mixture was purified via column chromatography on silica gel (2% MeOH in CH₂Cl₂) to isolate AA-13 (29 mg, 73%) as a colorless solid: ¹H NMR (400 MHz, CDCl₃) δ 8.90 (d, J=23.6 Hz, 1H), 7.75 (s, 4H), 7.39 (q, J=8.0 Hz, 1H), 7.33-7.16 (m, 5H), 6.86 (d, J=8.4 Hz, 1H), 6.67 (dd, J=13.3, 8.2 Hz, 1H), 5.23 (d, J=9.5 Hz, 1H), 5.15 (t, J=5.5 Hz, 1H), 3.96-3.66 (m, 6H), 3.51 (s, 1H), 3.25 (dd, J=15.1, 9.1 Hz, 1H), 3.01-2.66 (m, 5H), 2.15 (dq, J=14.5, 6.3 Hz, 1H), 1.98 (dt, J=12.9, 5.5 Hz, 1H), 1.59 (dd, J=14.1, 7.2 Hz, 1H), 0.80 (q, J=8.3 Hz, 6H); ¹³C NMR (101 MHz, CDCl₃) δ 166.57, 163.95, 162.07, 159.63, 156.13, 140.64, 137.66, 134.76, 134.62, 134.24, 129.22, 128.49, 128.43, 126.49, 121.02, 115.25, 105.99, 105.73, 103.30, 75.32, 73.20, 69.54, 66.88, 58.69, 54.82, 54.48, 38.74, 32.70, 27.16, 19.92, 19.56; HRMS (ESI) m/z [M+H] calcd C₃₂H₃₈FN₃O₈S 666.22559; found 666.2269.

### Scheme7: Synthetic Scheme Towards the Synthesis of Compound AA14 and Compound AA-15

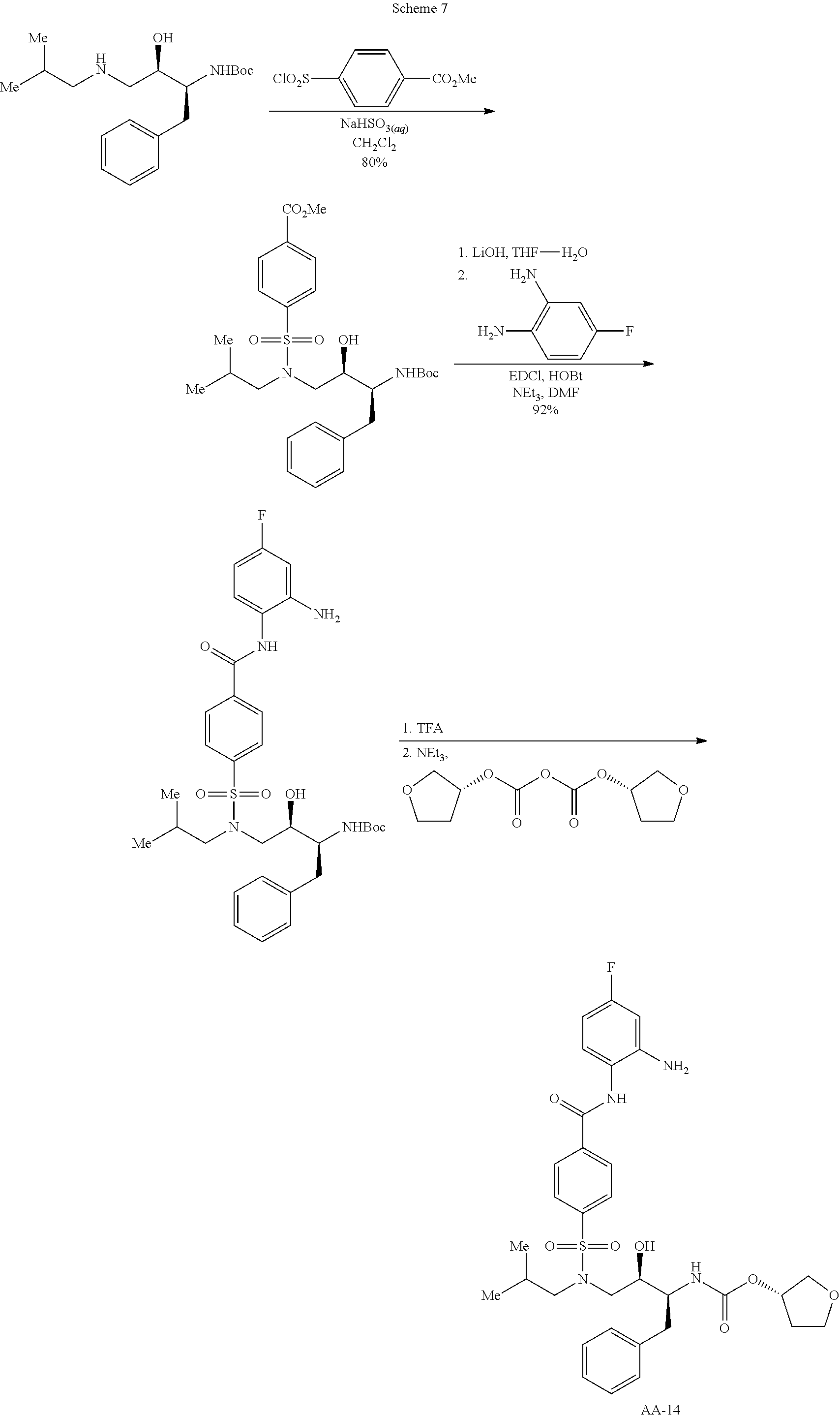

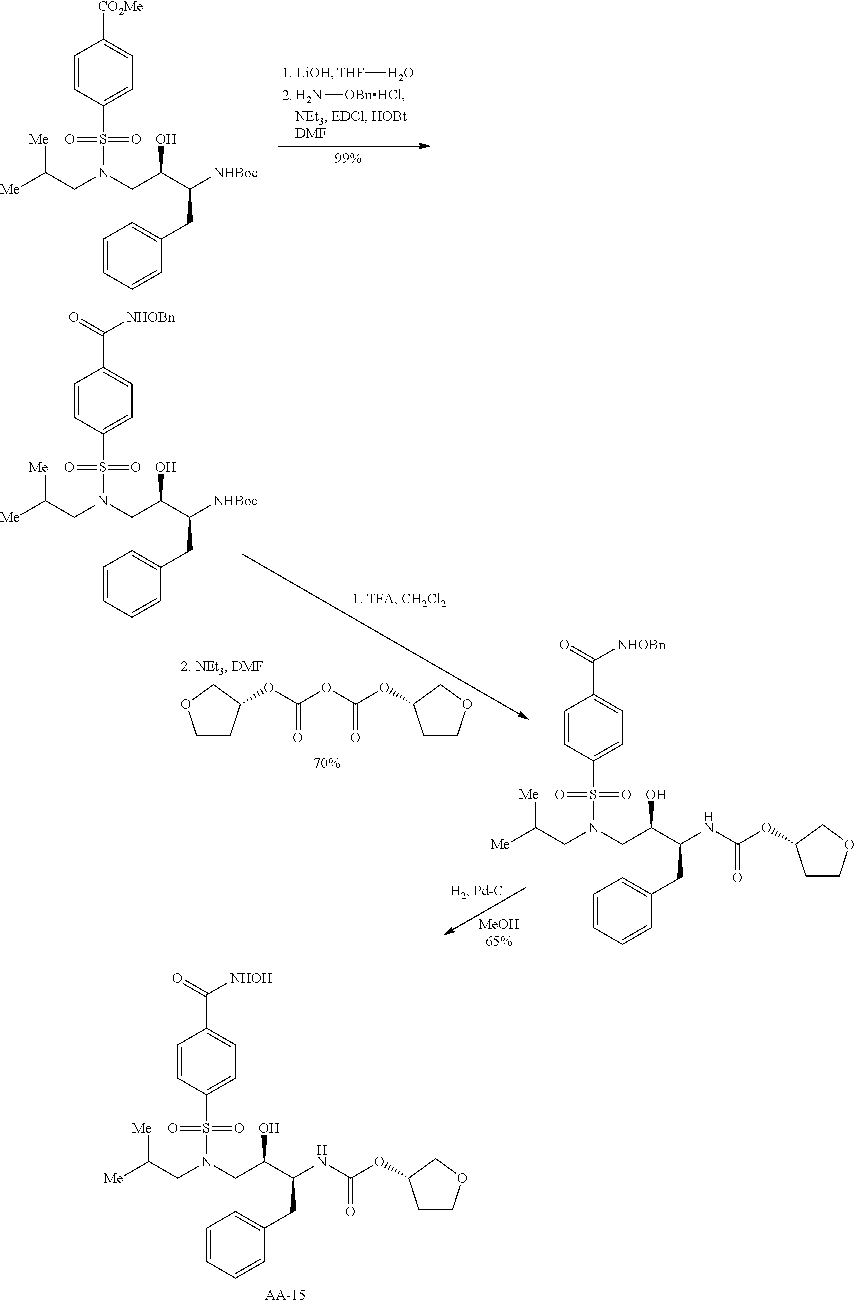

### Example 20

### Methyl 4-(N-((2R,3S)-3-((tert-butoxycarbonyl)amino)-2-hydroxy-4-phenylbutyl)-N-isobutylsulfamoyl)benzoate (22)

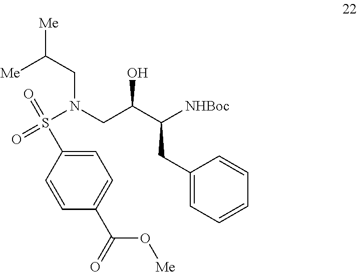 [SMILES: COC(=O)c1ccc(S(=O)(=O)N(CC(C)C)C[C@@H](O)[C@H](Cc2ccccc2)NC(=O)OC(C)(C)C)cc1]

To a solution of amine 2 (1.359 g, 4.01 mmol, 1 eq) in CH₂Cl₂ (40.4 ml, 0.05 M) was added a saturated aqueous solution of sodium bicarbonate (40.4 ml, 0.05 M) followed by 4-nitrobenzenesulfonyl chloride (1.422 g, 6.01 mmol, 1.5 eq) and the resulting mixture was stirred overnight at room temperature. The mixture was extracted with CH₂Cl₂ and dried over anhydrous MgSO₄, followed by the removal of the solvent under reduced pressure and purification by column chromatography on silica gel (1% MeOH in CH₂Cl₂) to obtain the sulfonamide 22 (1.712 g, 80%) as a colorless amorphous solid: ¹H NMR (400 MHz, CDCl₃) δ 8.17-8.06 (m, 2H), 7.83-7.74 (m, 2H), 7.21 (tq, J=11.6, 7.1 Hz, 5H), 5.14-4.94 (m, 1H), 3.93 (d, J=12.7 Hz, 3H), 3.76-3.61 (m, 2H), 3.45 (s, 1H), 2.90 (tdd, J=22.0, 11.1, 5.5 Hz, 4H), 1.61-1.54 (m, 1H), 1.38 (d, J=9.1 Hz, 9H), 0.74 (dd, J=9.6, 6.4 Hz, 6H); ¹³C NMR (101 MHz, CDCl₃) δ 165.43, 156.02, 142.33, 137.92, 133.74, 130.24, 129.19, 128.37, 127.12, 126.35, 79.49, 77.27, 76.96, 76.64, 69.56, 58.34, 54.20, 52.60, 38.52, 28.22, 27.07, 19.88, 19.53.

### Example 21

### Tert-butyl ((2S,3R)-4-((4-((2-amino-4-fluorophenyl)carbamoyl)-N-isobutylphenyl)su-lfonamido)-3-hydroxy-1-phenylbutan-2-yl)carbamate (23)

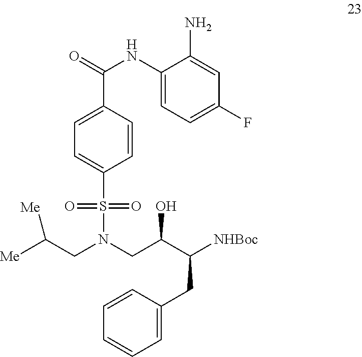 [SMILES: CC(C)CN(C[C@@H](O)[C@H](Cc1ccccc1)NC(=O)OC(C)(C)C)S(=O)(=O)c1ccc(C(=O)Nc2ccc(F)cc2N)cc1]

To a solution of ester 22 (1 eq) in THF/water (0.5 M) was added LiOH·H₂O (1.5 eq). The reaction was stirred for an hour after which 1 M HCl was added to it to quench the reaction. EtOAc was used to extract the acid in the organic layer, which was subsequently washed with brine before drying over MgSO₄. The organic layer was concentrated under reduced pressure to yield the crude acid. This crude acid was then dissolved in DMF (0.2 M). HATU (1.1 eq), and diisopropylethylamine (3 eq) were added to this solution and stirred for 5 minutes. After 5 minutes 4-fluorobenzene-1,2-diamine (1.1 eq) was added to this mixture and stirred overnight. The reaction was quenched with 1 M HCl and extracted with EtOAc thrice. The combined organic layer was washed with saturated sodium bicarbonate and brine and dried over anhydrous MgSO₄. The solvent was evaporated under reduced pressure and purified using column chromatography on silica gel (2% MeOH in CH₂Cl₂) to obtain compound 23 in 92% yield as a yellow solid: ¹H NMR (400 MHz, CDCl₃) δ 8.50 (d, J=12.4 Hz, 1H), 7.94 (d, J=8.7 Hz, 2H), 7.71 (d, J=7.9 Hz, 2H), 7.25 (d, J=7.3 Hz, 2H), 7.17 (q, J=9.0 Hz, 4H), 6.45 (td, J=11.8, 5.8 Hz, 2H), 5.07-5.00 (m, 1H), 3.77-3.70 (m, 1H), 3.65 (q, J=8.3 Hz, 1H), 3.24 (dd, J=15.1, 8.9 Hz, 1H), 2.96-2.68 (m, 8H), 1.67-1.52 (m, J=7.7, 7.3 Hz, 1H), 1.37 (s, 7H), 1.31 (s, 1H), 1.23 (q, J=6.0 Hz, 1H), 0.83-0.73 (m, 6H); ¹³C NMR (101 MHz, CDCl₃) δ 164.78, 163.14, 156.08, 143.29, 141.31, 137.89, 129.17, 128.39, 128.29, 127.28, 126.38, 119.20, 105.48, 104.17, 79.59, 69.58, 58.19, 54.26, 53.96, 38.49, 36.45, 31.37, 28.22, 27.01, 19.86, 19.59.

### Example 22

### (S)-Tetrahydrofuran-3-yl ((2S,3R)-4-((4-((2-amino-4-fluorophenyl)carbamoyl)-N-isob-utylphenyl)sulfonamido)-3-hydroxy-1-phenylbutan-2-yl)carbamate (AA-14)

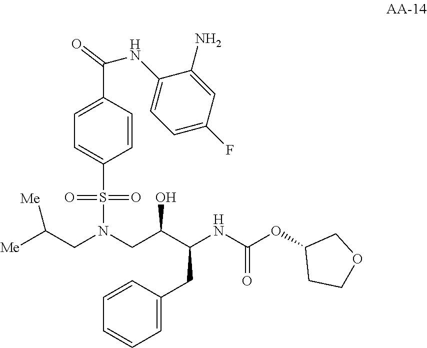 [SMILES: CC(C)CN(C[C@@H](O)[C@H](Cc1ccccc1)NC(=O)O[C@H]1CCOC1)S(=O)(=O)c1ccc(C(=O)Nc2ccc(F)cc2N)cc1]

Stirring amine 23 (1 eq) in a 0.5 M solution of of trifluoroacetic acid in CH₂Cl₂ for 1 hour resulted in Boc group deprotection. The solvent was evaporated under reduced pressure and the crude mixture was dissolved in CH₂Cl₂ (0.02 M). To this solution triethylamine (10 eq) and compound 17 (1.1 eq) were added. This mixture was stirred for 3 hours. The reaction mixture was concentrated, and the crude product was purified by column chromatography over silica gel (2% MeOH in CH₂Cl₂) to provide AA-14 in 67% yield as an off-white solid: ¹H NMR (500 MHz, CDCl₃) δ 8.13 (s, 1H), 7.96 (d, J=8.0 Hz, 2H), 7.76 (d, J=8.0 Hz, 2H), 7.29 (t, J=7.4 Hz, 2H), 7.20 (td, J=14.0, 6.7 Hz, 4H), 6.51 (tt, J=8.5, 4.2 Hz, 2H), 5.31-5.23 (m, 1H), 5.16-5.10 (m, 1H), 3.88 (p, J=7.2 Hz, 1H), 3.79 (dq, J=12.0, 4.4 Hz, 3H), 3.70 (h, J=8.1 Hz, 2H), 3.46 (s, 1H), 3.23 (dd, J=15.1, 8.9 Hz, 1H), 2.97-2.86 (m, 3H), 2.85 (d, J=10.2 Hz, 1H), 2.76 (dd, J=13.5, 6.8 Hz, 1H), 2.16-2.07 (m, 1H), 1.95 (dt, J=13.5, 6.0 Hz, 1H), 1.60 (dp, J=13.4, 6.7 Hz, 1H), 1.31-1.23 (m, 1H), 0.85-0.75 (m, 6H); ¹³C NMR (126 MHz, CDCl₃) δ 164.71, 163.08, 161.13, 156.34, 143.19, 143.11, 141.46, 137.96, 137.69, 129.30, 128.61, 128.32, 127.56, 127.45, 127.37, 126.70, 119.36, 106.14, 105.96, 104.59, 104.38, 75.53, 73.30, 69.63, 67.00, 58.53, 55.71, 54.99, 54.29, 39.79, 38.73, 32.77, 27.19, 19.99, 19.70.

### Example 23

### Tert-butyl ((2S,3R)-4-((4-((benzyloxy)carbamoyl)-N-isobutylphenyl)sulfonamido)-3-hydroxy-1-phenylbutan-2-yl)carbamate (24)

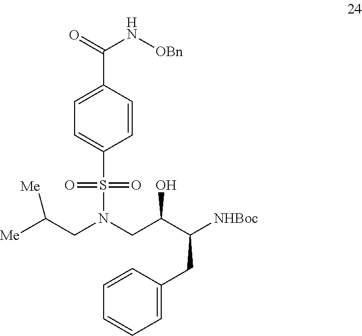 [SMILES: CC(C)CN(C[C@@H](O)[C@H](Cc1ccccc1)NC(=O)OC(C)(C)C)S(=O)(=O)c1ccc(C(=O)NOCc2ccccc2)cc1]

To a solution of ester 22 (1 eq) in THF/water (0.5 M) was added LiOH·H₂O (1.5 eq). The reaction was stirred for an hour after which 1 M HCl was added to it to quench the reaction. EtOAc was used to extract the acid into the organic layer, which was subsequently washed with brine before drying over MgSO₄. The organic layer was concentrated under reduced pressure to yield the crude acid was dissolved in DMF (0.2 M). HATU (1.1 eq), and diisopropylethylamine (3 eq) were added to this solution and stirred for 5 minutes. A solution of triethylamine (1.2 eq) and O-benzylhydroxylamine hydrochloride (1.1 eq) in DMF were added to this mixture and stirred overnight. The reaction was quenched with 1 M HCl and extracted with EtOAc thrice. The combined organic layer was washed with saturated sodium bicarbonate and brine and dried over anhydrous MgSO₄. The solvent was evaporated under reduced pressure and purified using column chromatography on silica gel (2% MeOH in CH₂Cl₂) to obtain compound 24 in 99% yield as a white solid: ¹H NMR (400 MHz, CDCl₃) δ 9.97 (s, 1H), 7.88 (d, J=13.5 Hz, 1H), 7.77 (d, J=8.2 Hz, 2H), 7.69 (d, J=8.1 Hz, 2H), 7.43-7.30 (m, 5H), 7.29-7.14 (m, 5H), 5.02 (d, J=9.3 Hz, 1H), 4.99 (s, 2H), 3.72 (d, J=8.7 Hz, 1H), 3.64 (q, J=8.3 Hz, 1H), 3.54 (s, 1H), 3.21 (dd, J=15.1, 9.1 Hz, 1H), 2.95-2.80 (m, 7H), 2.75 (s, 1H), 2.69 (dd, J=13.4, 6.7 Hz, 1H), 1.57 (dq, J=14.9, 7.5 Hz, 1H), 1.36 (s, 9H), 0.75 (dd, J=6.6, 4.6 Hz, 6H); ¹³C NMR (101 MHz, CDCl₃) δ 162.57, 156.06, 141.35, 137.90, 135.91, 135.01, 129.22, 129.17, 128.74, 128.51, 128.37, 128.05, 127.26, 126.36, 79.56, 78.28, 69.51, 58.26, 54.24, 54.04, 38.51, 36.47, 31.38, 28.22, 27.02, 19.86, 19.57.

### Example 24

### (S)-tetrahydrofuran-3-yl ((2S,3R)-4-((4-((benzyloxy)carbamoyl)-N-isobutylphenyl)s-ulfonamido)-3-hydroxy-1-phenylbutan-2-yl)carbamate (25)

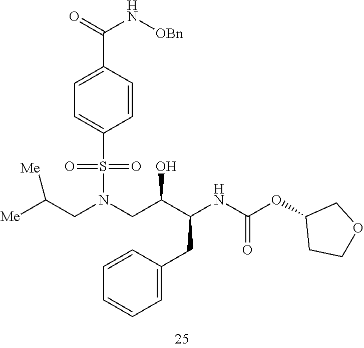 [SMILES: CC(C)CN(C[C@@H](O)[C@H](Cc1ccccc1)NC(=O)O[C@H]1CCOC1)S(=O)(=O)c1ccc(C(=O)NOCc2ccccc2)cc1]

Stirring carbamate 24 (1 eq) in a 0.5 M solution of trifluoroacetic acid in CH₂Cl₂ for 1 hour afforded deprotection of the Boc group. The solvent was evaporated under reduced pressure and the crude mixture was redissolved in CH₂Cl₂ (0.02 M). To this solution triethylamine (10 eq) and compound 17 (1.1 eq) were added, and the mixture was stirred for 3 hours. The reaction mixture was concentrated, and the crude purified by column chromatography over silica gel (2% MeOH in CH₂Cl₂) to provide compound 25 as a colorless solid in 70% yield: ¹H NMR (500 MHz, CDCl3) δ 9.71 (s, 1H), 7.76-7.66 (m, 4H), 7.43-7.29 (m, 5H), 7.26 (t, J=7.3 Hz, 2H), 7.18 (h, J=7.1 Hz, 3H), 5.33-5.18 (m, 1H), 5.09 (p, J=5.9 Hz, 1H), 4.99 (s, 2H), 3.84 (q, J=7.9 Hz, 1H), 3.77 (d, J=4.0 Hz, 1H), 3.76-3.58 (m, 4H), 3.52 (s, 1H), 3.19 (dd, J=15.2, 8.8 Hz, 1H), 2.89 (dq, J=13.2, 7.0 Hz, 3H), 2.85-2.79 (m, 1H), 2.78-2.68 (m, 1H), 2.08 (dp, J=15.1, 7.2 Hz, 1H), 1.92 (dt, J=12.6, 5.5 Hz, 1H), 1.59 (dq, J=13.8, 6.7 Hz, 1H), 0.82-0.73 (m, 6H); ¹³C NMR (126 MHz, CDCl3) δ 164.80, 156.32, 155.87, 141.55, 141.09, 137.90, 137.72, 136.21, 136.02, 135.04, 133.69, 129.33, 129.30, 128.96, 128.69, 128.65, 128.57, 128.42, 128.16, 127.43, 127.19, 126.66, 126.51, 78.48, 76.71, 75.66, 75.47, 75.39, 73.24, 69.63, 69.53, 66.96, 58.64, 58.33, 55.71, 54.97, 54.34, 54.08, 39.71, 38.80, 38.71, 32.74, 32.58, 29.71, 27.34, 27.22, 27.13, 19.97, 19.72.

### Example 25

### (S)-tetrahydrofuran-3-yl ((2S,3R)-3-hydroxy-4-((4-(hydroxycarbamoyl)-N-isobutylp-henyl)sulfonamido)-1-phenylbutan-2-yl)carbamate (AA-15)

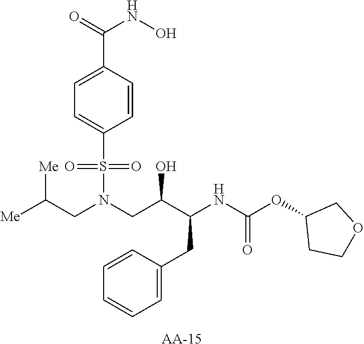

To a solution of compound 25 (21 mg, 0.033 mmol) in MeOH (0.33 ml, 0.1 M) was added 10% Pd/C (2 mg) and stirred overnight under a hydrogen atmosphere at balloon pressure. The reaction mixture was filtered over Celite, and the filter cake was washed with MeOH. Solvent was removed from the filtrate under reduced pressure and the crude residue was purified by column chromatography on silica gel (3% MeOH in CH₂Cl₂) to obtain diamine AA-15 (12 mg, 65%) as a colorless solid: ¹H NMR (500 MHz, CDCl₃) δ 7.92 (d, J=8.1 Hz, 2H), 7.81 (d, J=8.1 Hz, 2H), 7.30 (t, J=7.5 Hz, 2H), 7.22 (t, J=7.3 Hz, 3H), 6.23 (s, 1H), 5.93 (s, 1H), 5.22 (d, J=9.4 Hz, 1H), 5.18-5.12 (m, 1H), 3.91 (q, J=7.9 Hz, 1H), 3.81 (dq, J=10.6, 5.2 Hz, 3H), 3.72 (dd, J=14.4, 9.4 Hz, 2H), 3.43 (s, 1H), 3.25 (dd, J=15.2, 9.0 Hz, 1H), 3.00-2.83 (m, 4H), 2.76 (dd, J=13.3, 6.6 Hz, 1H), 2.19-2.08 (m, 1H), 1.97 (dt, J=12.8, 5.7 Hz, 1H), 1.58 (dt, J=13.5, 6.9 Hz, 1H), 0.79 (t, J=6.3 Hz, 6H); ¹³C NMR (126 MHz, CDCl₃) δ 167.58, 156.29, 141.55, 137.70, 137.39, 129.32, 128.60, 128.29, 127.59, 126.68, 75.51, 73.32, 69.64, 67.00, 58.66, 54.99, 54.47, 38.79, 32.79, 27.23, 20.00, 19.67.

### Example 26

### Methyl 3-((2-amino-4-fluorophenyl)carbamoyl)benzoate (26)

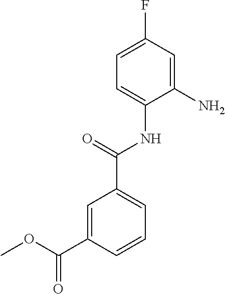 [SMILES: COC(=O)c1cccc(C(=O)Nc2ccc(F)cc2N)c1]

4-fluorobenzene-1,2-diamine (0.45 mmol, 0.9 eq.) was dissolved in acetonitrile (4 mL) and stirred in a 20 mL vial. 3-(Methoxycarbonyl)benzoic acid (0.5 mmol, 1 eq.), EDC·HCl (0.5 mmol, 1 eq.), HOBt. H₂O (0.05 mmol, 0.1 eq.), DMAP (0.5 mmol, 1 eq.), and DIPEA (2.5 mmol, 5 eq.) were added in sequence to the reaction mixture and stirred at room temperature for 16-36 hours. Upon reaction completion, the reaction mixture was diluted with water and extracted with ethyl acetate. The organic layer was isolated and washed with 1 M HCl solution (2×15 mL), saturated NaHCO₃ (2×15 mL), and brine (2×15 mL), dried with sodium sulfate, and concentrated under reduced pressure. The crude product was purified via column chromatography (EtOAc/hexanes [10:90 to 70:30]) to afford the pure compound 26. Pale yellow solid (0.176 g, 60.9% yield). ¹H NMR (800 MHz, DMSO) δ 9.81 (s, 1H), 8.57 (s, 1H), 8.26 (d, J=7.7 Hz, 1H), 8.15 (d, J=7.7 Hz, 1H), 7.68 (t, J=7.7 Hz, 1H), 7.14-7.10 (m, 1H), 6.55 (dd, J=11.3, 2.9 Hz, 1H), 6.37 (td, J=8.5, 2.9 Hz, 1H), 5.28 (s, 2H), 3.91 (s, 3H). ¹³C NMR (201 MHz, DMSO) δ 166.32, 165.18, 161.66 (d, J=238.8 Hz), 146.15 (d, J=12.21 Hz), 135.56, 132.92, 132.28, 130.20, 129.33, 129.24, 129.19, 129.08, 119.39, 102.43 (d, J=22.89 Hz), 101.84 (d, J=25.18 Hz), 52.83.

### Example 27

### 3-((2-Amino-4-fluorophenyl)carbamoyl)benzoic acid (27)

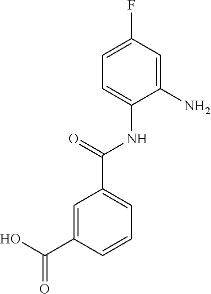 [SMILES: Nc1cc(F)ccc1NC(=O)c1cccc(C(=O)O)c1]

Compound 26 was dissolved in 8 mL THF in a 20 mL vial and stirred. To the reaction mixture, 1.5 mL aq. LiOH solution was added dropwise and stirred overnight. Upon reaction conclusion, the mixture was diluted with water, neutralized with HCl, extracted with ethyl acetate, and dried over sodium sulfate. The dried organic layer was concentrated under reduced pressure to afford the pure compound 27. Brown powder (0.133 g, quant. yield). ¹H NMR (800 MHz, DMSO) δ 9.79 (s, 1H), 8.56 (s, 1H), 8.22 (d, J=7.7 Hz, 1H), 8.13 (d, J=7.6 Hz, 1H), 7.65 (t, J=7.7 Hz, 1H), 7.12 (dd, J=8.6, 6.3 Hz, 1H), 6.55 (dd, J=11.2, 2.9 Hz, 1H), 6.37 (td, J=8.4, 2.9 Hz, 1H). ¹³C NMR (201 MHz, DMSO) δ 167.39, 165.36, 161.62 (d, J=239.56 Hz), 146.09 (d, J=12.2 Hz), 135.41, 133.85, 132.48 (d, J=12.97 Hz), 131.40, 129.23, 129.19, 129.13, 119.49, 112.49, 102.45 (d, J=22.9 Hz), 101.86 (d, J=25.2 Hz).

### Example 28

### N¹-(2-Amino-4-fluorophenyl)-N³-((2S,3R)-3-hydroxy-4-((N-isobutyl-4-nitrophenyl)sulfonamido)-1-phenylbutan-2-yl)isophthalamide (28)

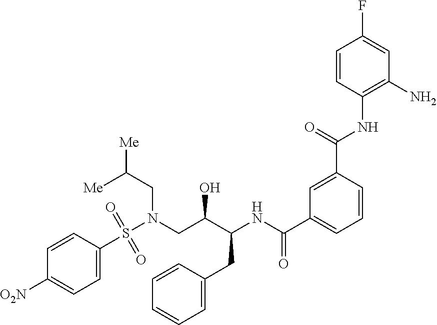 [SMILES: CC(C)CN(C[C@@H](O)[C@H](Cc1ccccc1)NC(=O)c1cccc(C(=O)Nc2ccc(F)cc2N)c1)S(=O)(=O)c1ccc([N+](=O)[O-])cc1]

N-((2R,3S)-3-Amino-2-hydroxy-4-phenylbutyl)-N-isobutyl-4-nitrobenzenesulfonamide (0.45 mmol, 0.9 eq.) was dissolved in acetonitrile (4 mL) and stirred in a 20 mL vial. 3-((2-amino-4-fluorophenyl)carbamoyl)benzoic acid (27) (0.5 mmol, 1 eq.), EDC·HCl (0.50 mmol, 1 eq.), HOBt·H₂O (0.050 mmol, 0.1 eq.), DMAP (0.50 mmol, 1 eq.), and DIPEA (2.5 mmol, 5 eq.) were added in sequence to the reaction mixture and stirred at room temperature for 16-36 hours. Upon reaction completion, the reaction mixture was diluted with water and extracted with ethyl acetate. The organic layer was isolated and washed with 1 M HCl solution (2×15 mL), saturated NaHCO₃ (2×15 mL), and brine (2×15 mL), dried with sodium sulfate, and concentrated under reduced pressure. The crude product was purified via column chromatography (EtOAc/hexanes [10:90 to 70:30]) to afford the pure compound 28. Yellow powder (0.048 g, 28.2% yield). ¹H NMR (800 MHz, DMSO) δ 9.68 (s, 1H), 8.43 (d, J=9.0 Hz, 1H), 8.33 (s, 1H), 8.29 (d, J=8.8 Hz, 2H), 8.13 (d, J=7.7 Hz, 1H), 8.02 (d, J=8.7 Hz, 2H), 7.91 (d, J=7.8 Hz, 1H), 7.59 (t, J=7.7 Hz, 1H), 7.26 (d, J=7.5 Hz, 2H), 7.21 (t, J=7.6 Hz, 2H), 7.13 (dt, J=15.0, 7.9 Hz, 2H), 6.56 (dd, J=11.2, 2.9 Hz, 1H), 6.38 (td, J=8.5, 2.9 Hz, 1H), 5.26 (s, 2H), 5.21 (d, J=6.9 Hz, 1H), 4.11 (q, J=10.6 Hz, 1H), 3.76 (q, J=9.0 Hz, 1H), 3.41 (dd, J=15.0, 2.7 Hz, 1H), 3.20 (dd, J=13.5, 8.7 Hz, 1H), 3.13 (dd, J=14.1, 3.2 Hz, 1H), 3.09 (dd, J=15.0, 8.9 Hz, 1H), 2.92 (dd, J=13.6, 6.3 Hz, 1H), 2.76 (dd, J=14.0, 11.2 Hz, 1H), 2.04-1.97 (m, 1H), 0.86 (d, J=6.6 Hz, 3H), 0.81 (d, J=6.7 Hz, 3H). ¹³C NMR (201 MHz, DMSO) δ 165.99, 165.68, 161.58 (d, J=239.56 Hz), 149.97, 145.97 (d, J=11.45 Hz), 145.44, 139.92, 135.18, 135.01, 130.70, 130.34, 129.52, 129.04, 128.99, 128.68, 128.46, 127.33, 126.29, 124.82, 119.54, 102.54 (d, J=22.89 Hz), 101.92 (d, J=24.41 Hz), 72.10, 56.20, 54.74, 52.10, 35.47, 26.34, 20.31, 20.20.

### Example 29

### N₁-(2-amino-4-fluorophenyl)-N³-((2S,3R)-4-((4-amino-N-isobutylphenyl)sulfonamido)-3-hydroxy-1-phenylbutan-2-yl)isophthalamide (NB-1)

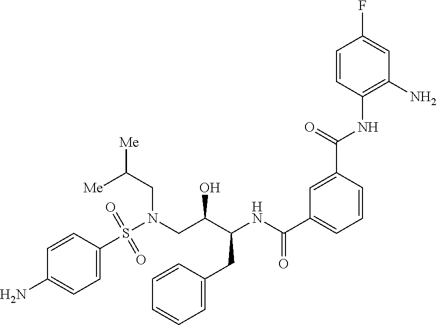 [SMILES: CC(C)CN(C[C@@H](O)[C@H](Cc1ccccc1)NC(=O)c1cccc(C(=O)Nc2ccc(F)cc2N)c1)S(=O)(=O)c1ccc(N)cc1]

Compound 28 was dissolved in methanol along with PtO₂ (0.1 eq.) and potassium carbonate (0.1 eq.) in a round-bottom flask that was then sealed. Air was purged from the reaction vessel using 2 hydrogen balloons, and a third hydrogen balloon was set up to stir overnight. Upon reaction completion, the mixture was filtered and purified via column chromatography (EtOAc/hexanes [40:60 to 70:30]) to afford pure compound NB-1. Pale yellow powder (0.0402 g, 52.6% yield). ¹H NMR (800 MHz, DMSO) δ 9.67 (s, 1H), 8.41 (d, J=9.0 Hz, 1H), 8.33 (s, 1H), 8.11 (d, J=7.7 Hz, 1H), 7.93 (d, J=7.8 Hz, 1H), 7.59 (t, J=7.7 Hz, 1H), 7.34 (d, J=8.4 Hz, 2H), 7.29 (d, J=7.6 Hz, 2H), 7.21 (t, J=7.5 Hz, 2H), 7.13 (dt, J=20.1, 7.7 Hz, 2H), 6.56 (dd, J=11.2, 2.9 Hz, 1H), 6.51 (d, J=8.5 Hz, 2H), 6.38 (dd, J=9.5, 6.8 Hz, 1H), 5.92 (s, 2H), 5.27 (s, 2H), 5.16 (d, J=6.6 Hz, 1H), 4.14 (t, J=9.8 Hz, 1H), 3.83 (s, 2H), 3.18 (d, J=13.0 Hz, 1H), 2.97 (dd, J=13.4, 8.6 Hz, 1H), 2.79 (t, J=12.6 Hz, 1H), 2.71 (dd, J=14.9, 8.2 Hz, 1H), 2.63 (dd, J=13.3, 6.1 Hz, 1H), 1.99 (dq, J=14.0, 6.6 Hz, 1H), 0.87 (d, J=6.5 Hz, 3H), 0.80 (d, J=6.7 Hz, 3H). ¹³C NMR (201 MHz, DMSO) δ 165.93, 165.77, 161.57 (d, J=239.56 Hz), 153.12, 145.97 (d, J=11.45 Hz), 140.22, 135.15 (d, J=9.92 Hz), 130.64, 130.41, 129.59, 129.48, 129.04 (d, J=10.68 Hz), 128.60, 128.41, 127.36, 126.20, 123.90, 119.56, 117.44, 113.11, 102.55 (d, J=22.89 Hz), 101.93 (d, J=25.18 Hz), 73.29, 57.91, 54.72, 53.45, 35.24, 26.81, 20.57.

### Example 30

### tert-Butyl 2-(4-(methoxycarbonyl)benzoyl)-1-propylhydrazine-1-carboxylate (29)

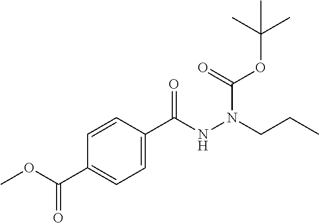 [SMILES: CCCN(NC(=O)c1ccc(C(=O)OC)cc1)C(=O)OC(C)(C)C]

A vial was charged with 4-(Methoxycarbonyl)benzoic acid (0.0901 g, 0.5 mmol) and HATU (0.2282 g, 1.2 eq.) and dissolved in DMF (4 mL). DIPEA was added dropwise and stirred for 15 minutes. After 15 minutes, tert-butyl 1-propylhydrazine-1-carboxylate (0.0875 mL, 0.9 eq.) was added to the reaction mixture and allowed to stir overnight. Upon reaction completion, the mixture was diluted with water and extracted into ethyl acetate. The organic layer was washed with 1M HCl (2×15 mL), sat. NaHCO₃ (2×15 mL), and brine (2×15 mL), dried with Na₂SO₄, and concentrated under reduced pressure. The crude mixture was purified via column chromatography (DCM/MeOH [100:0 to 97:3]). Clear-yellow oil (120.2 g, 71.5% yield). ¹H NMR (800 MHz, DMSO) δ 10.71 (s, 1H), 8.07 (d, J=8.2 Hz, 2H), 7.98 (d, J=8.0 Hz, 1H), 7.93 (d, J=7.9 Hz, 1H), 3.89 (s, 3H), 3.41 (s, 2H), 1.55-1.49 (m, 2H), 1.45 (s, 4H), 1.34 (s, 5H), 0.89 (d, J=7.2 Hz, 3H). ¹³C NMR (201 MHz, DMSO) δ 166.09, 165.43, 155.21, 137.37, 132.78, 129.78, 128.25, 80.05, 52.90, 50.53, 28.28, 20.91, 11.63.

### Example 31

### 4-(2-(tert-Butoxycarbonyl)-2-propylhydrazine-1-carbonyl)benzoic acid (30)

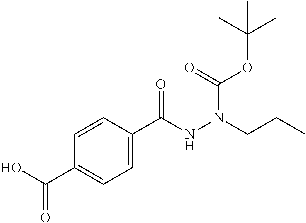 [SMILES: CCCN(NC(=O)c1ccc(C(=O)O)cc1)C(=O)OC(C)(C)C]

Compound 29 was dissolved in 8 mL THF in a 20 mL vial and stirred. To the reaction mixture, 1.5 mL aq. LiOH solution was added dropwise and stirred overnight. Upon reaction conclusion, the mixture was diluted with water, neutralized with HCl, extracted with ethyl acetate, and dried with sodium sulfate. The dried organic layer was concentrated under reduced pressure to afford the pure compound 30. Pale yellow powder (0.098 g, quant. yield). ¹H NMR (800 MHz, DMSO) δ 10.68 (s, 1H), 8.05 (d, J=8.0 Hz, 2H), 7.96 (d, J=7.9 Hz, 1H), 7.91 (d, J=8.0 Hz, 1H), 3.41 (s, 2H), 1.53 (d, J=6.8 Hz, 1H), 1.45 (s, 4H), 1.35 (d, J=14.2 Hz, 5H), 0.89 (d, J=7.1 Hz, 3H). ¹³C NMR (201 MHz, DMSO) δ 167.16, 165.55, 155.24, 137.01, 134.07, 129.87, 128.08, 80.03, 50.53, 28.29, 20.91, 11.64.

### Example 32

### tert-Butyl 2-(4-(((2S,3R)-3-hydroxy-4-((N-isobutyl-4-nitrophenyl)sulfonamido)-1-phenylbutan-2-yl)carbamoyl)benzoyl)-1-propylhydrazine-1-carboxylate (31)

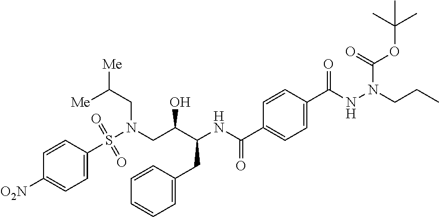 [SMILES: CCCN(NC(=O)c1ccc(C(=O)N[C@@H](Cc2ccccc2)[C@H](O)CN(CC(C)C)S(=O)(=O)c2ccc([N+](=O)[O-])cc2)cc1)C(=O)OC(C)(C)C]

Compound 30 (0.45 mmol, 0.9 eq.) was dissolved in acetonitrile (4 mL) and stirred in a 20 mL vial. 4-(2-(tert-butoxycarbonyl)-2-propylhydrazine-1-carbonyl)benzoic acid (0.5 mmol, 1 eq.), EDC·HCl (0.5 mmol, 1 eq.), HOBt·H₂O (0.05 mmol, 0.1 eq.), DMAP (0.5 mmol, 1 eq.), and DIPEA (2.5 mmol, 5 eq.) were added in sequence to the reaction mixture and stirred at room temperature for 16-36 hours. Upon reaction completion, the reaction mixture was diluted with water and extracted with ethyl acetate. The organic layer was isolated and washed with 1 M HCl solution (2×15 mL), saturated NaHCO₃ (2×15 mL), and brine (2×15 mL), dried with sodium sulfate, and concentrated under reduced pressure. The crude product was purified via column chromatography (EtOAc/hexanes [10:90 to 70:30]) to afford pure compound 31. Off-white powder (0.089 g, 48.9% yield). ¹H NMR (800 MHz, DMSO) δ 10.61 (d, J=11.7 Hz, 1H), 8.41 (d, J=9.0 Hz, 1H), 8.30 (d, J=8.3 Hz, 2H), 8.01 (d, J=8.3 Hz, 2H), 7.92-7.85 (m, 2H), 7.81 (d, J=7.9 Hz, 2H), 7.25 (d, J=7.6 Hz, 2H), 7.21 (t, J=7.5 Hz, 2H), 7.11 (t, J=7.3 Hz, 1H), 5.20 (d, J=6.9 Hz, 1H), 4.06 (t, J=9.9 Hz, 1H), 3.73 (d, J=7.6 Hz, 1H), 3.41 (d, J=14.6 Hz, 2H), 3.19 (dd, J=13.6, 8.6 Hz, 1H), 3.11 (t, J=13.0 Hz, 2H), 2.92 (dd, J=13.5, 6.3 Hz, 1H), 2.75 (t, J=12.6 Hz, 1H), 2.04-1.96 (m, 1H), 1.57-1.50 (m, 2H), 1.45 (s, 4H), 1.33 (s, 5H), 0.89 (d, J=7.5 Hz, 3H), 0.85 (d, J=6.6 Hz, 3H), 0.80 (d, J=6.6 Hz, 3H). ¹³C NMR (201 MHz, DMSO) δ 165.79, 165.55, 155.27, 154.69, 149.97, 145.55, 139.89, 137.71, 135.46, 129.53, 129.01, 128.44, 127.73, 126.30, 124.82, 80.56, 79.99, 72.14, 56.11, 54.84, 52.09, 50.55, 35.44, 28.27, 26.32, 20.18, 11.66.

### Example 33

### tert-Butyl 2-(4-(((2S,3R)-4-((4-amino-N-isobutylphenyl)sulfonamido)-3-hydroxy-1-phenylbutan-2-yl)carbamoyl)benzoyl)-1-propylhydrazine-1-carboxylate (32)

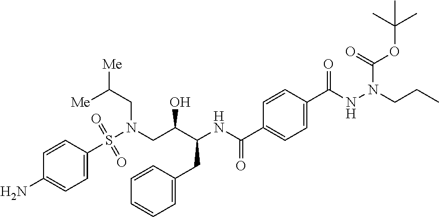 [SMILES: CCCN(NC(=O)c1ccc(C(=O)N[C@@H](Cc2ccccc2)[C@H](O)CN(CC(C)C)S(=O)(=O)c2ccc(N)cc2)cc1)C(=O)OC(C)(C)C]

Compound 31 was dissolved in methanol along with PtO₂ (0.1 eq.) and potassium carbonate (0.1 eq.) in a round-bottom flask that was then sealed. Air was purged from the reaction vessel using 2 hydrogen balloons, and a third hydrogen balloon was set up to stir overnight. Upon reaction completion, the mixture was filtered and purified via column chromatography (EtOAc/hexanes [40:60 to 70:30]) to afford pure compound 32. Pale yellow powder (0.066 g, 98% yield). ¹H NMR (800 MHz, DMSO) δ 10.61 (d, J=12.8 Hz, 1H), 8.43 (d, J=8.9 Hz, 1H), 7.91 (s, 1H), 7.86 (d, J=7.9 Hz, 1H), 7.82 (d, J=7.9 Hz, 2H), 7.30 (d, J=8.4 Hz, 2H), 7.27 (d, J=7.6 Hz, 2H), 7.21 (t, J=7.5 Hz, 2H), 7.11 (t, J=7.4 Hz, 1H), 6.52 (d, J=8.3 Hz, 2H), 5.94 (s, 2H), 5.17 (s, 1H), 4.09 (s, 2H), 3.80 (s, 1H), 3.41 (s, 1H), 3.36 (s, 1H), 3.17 (d, J=13.1 Hz, 1H), 2.97 (dd, J=13.3, 8.8 Hz, 1H), 2.77 (t, J=12.6 Hz, 1H), 2.73-2.66 (m, OH), 2.62 (dd, J=13.4, 6.1 Hz, 1H), 1.99 (dt, J=14.1, 7.0 Hz, 1H), 1.57-1.50 (m, 2H), 1.45 (s, 4H), 1.34 (s, 5H), 0.91-0.88 (m, 5H), 0.87 (d, J=6.5 Hz, 3H), 0.79 (d, J=6.6 Hz, 3H). ¹³C NMR (201 MHz, DMSO) δ 165.76, 162.52, 155.27, 153.14, 140.20, 137.96, 135.37, 135.06, 129.60, 129.44, 128.38, 127.79, 126.20, 123.92, 113.08, 80.58, 80.00, 73.45, 57.86, 54.82, 53.51, 50.56, 35.31, 28.29, 26.78, 20.56, 11.66.

### Example 34

### N-((2S,3R)-4-((4-amino-N-isobutylphenyl)sulfonamido)-3-hydroxy-1-phenylbutan-2-yl)-4-(2-propylhydrazine-1-carbonyl)benzamide (NB-3)

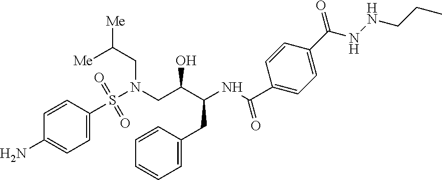 [SMILES: CCCNNC(=O)c1ccc(C(=O)N[C@@H](Cc2ccccc2)[C@H](O)CN(CC(C)C)S(=O)(=O)c2ccc(N)cc2)cc1]

Compound 32 was dissolved in 4 mL of ethyl acetate and stirred. 4 drops of concentrated HCl was added to the mixture, and allowed to stir for 12 hours. The mixture was diluted with water and extracted into ethyl acetate. The organic layer was dried with sodium sulfate and concentrated under reduced pressure. The residue was then purified via column chromatography (EtOAc/hexanes [70:30 to 100:0]). Off-white powder (0.0093 g, 52.7%). ¹H NMR (800 MHz, DMSO) δ 10.09 (s, 1H), 8.39 (d, J=9.2 Hz, 1H), 7.87 (d, J=8.2 Hz, 2H), 7.79 (d, J=8.1 Hz, 2H), 7.29 (d, J=8.3 Hz, 2H), 7.26 (d, J=7.2 Hz, 2H), 7.20 (t, J=7.6 Hz, 2H), 7.11 (t, J=7.6 Hz, 1H), 6.50 (d, J=8.2 Hz, 2H), 5.94 (s, 2H), 5.14 (d, J=6.9 Hz, 1H), 4.07 (d, J=9.2 Hz, 1H), 3.79 (s, 1H), 3.25 (s, 1H), 3.17 (d, J=13.7 Hz, 1H), 2.97 (t, J=11.1 Hz, 1H), 2.77 (d, J=9.6 Hz, 3H), 2.68 (dd, J=14.9, 8.2 Hz, 1H), 2.62 (dd, J=13.4, 6.1 Hz, 1H), 1.99 (d, J=7.2 Hz, 1H), 1.48 (q, J=7.4 Hz, 2H), 1.24 (s, 1H), 0.92 (t, J=7.6 Hz, 3H), 0.87 (d, J=6.5 Hz, 3H), 0.79 (d, J=6.6 Hz, 3H). ¹³C NMR (201 MHz, DMSO) δ 165.81, 165.12, 153.14, 140.20, 137.37, 135.86, 129.58, 129.44, 128.38, 127.63, 127.33, 126.20, 123.87, 113.07, 73.49, 57.91, 55.79, 54.82, 53.51, 35.38, 26.77, 21.32, 20.56, 12.13.

### Example 35

### Methyl 6-((2-amino-4-fluorophenyl)carbamoyl)nicotinate (33)

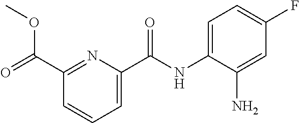 [SMILES: COC(=O)c1cccc(C(=O)Nc2ccc(F)cc2N)n1]

4-Fluorobenzene-1,2-diamine (0.45 mmol, 0.9 eq.) was dissolved in acetonitrile (4 mL) and stirred in a 20 mL vial. 5-(methoxycarbonyl)picolinic acid (0.5 mmol, 1 eq.), EDC·HCl (0.5 mmol, 1 eq.), HOBt-H₂O (0.05 mmol, 0.1 eq.), DMAP (0.5 mmol, 1 eq.), and DIPEA (2.5 mmol, 5 eq.) were added in sequence to the reaction mixture and stirred at room temperature for 16-36 hours. Upon reaction completion, the reaction mixture was diluted with water and extracted with ethyl acetate. The organic layer was isolated and washed with 1 M HCl solution (2×15 mL), saturated NaHCO₃ (2×15 mL), and brine (2×15 mL), dried with sodium sulfate, and concentrated under reduced pressure. The crude product was purified via column chromatography (EtOAc/hexanes [10:90 to 70:30]) to afford pure compound 33. Orange solid (0.130 g, 45.0%). ¹H NMR (400 MHz, DMSO) δ 10.08 (s, 1H), 9.15 (d, J=1.8 Hz, 1H), 8.50 (dd, J=8.2, 2.1 Hz, 1H), 8.23 (d, J=8.2 Hz, 1H), 7.28 (dd, J=8.7, 6.3 Hz, 1H), 6.55 (dd, J=11.1, 2.9 Hz, 1H), 6.38 (td, J=8.5, 2.9 Hz, 1H), 5.24 (s, 2H), 3.92 (s, 3H). ¹³C NMR (101 MHz, DMSO) δ 165.05, 162.24, 161.27 (d, J=239.83 Hz), 153.59, 149.17, 145.23 (d, J=11.66 Hz), 139.07, 128.09, 127.68, 122.76, 119.48, 102.77 (d, J=22.60 Hz), 102.27 (d, J=24.99 Hz), 53.10.

### Example 36

### 6-((2-Amino-4-fluorophenyl)carbamoyl)nicotinic acid (34)

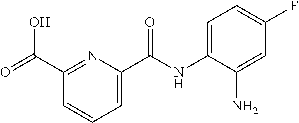 [SMILES: Nc1cc(F)ccc1NC(=O)c1cccc(C(=O)O)n1]

Methyl 6-((2-amino-4-fluorophenyl)carbamoyl)nicotinate (33) was dissolved in 8 mL THF in a 20 mL vial and stirred. To the reaction mixture, 1.5 mL aq. LiOH solution was added dropwise and stirred overnight. Upon reaction conclusion, the mixture was diluted with water, neutralized with HCl, extracted with ethyl acetate, and dried with sodium sulfate. The dried organic layer was concentrated under reduced pressure to afford the pure compound 34. Tan powder (0.127 g, 85.3% yield). ¹H NMR (800 MHz, DMSO) δ 10.10 (s, 1H), 9.16 (d, J=1.7 Hz, 1H), 8.50 (dd, J=8.1, 2.0 Hz, 1H), 8.23 (d, J=8.0 Hz, 1H), 7.32 (dd, J=8.6, 6.3 Hz, 1H), 6.58 (dd, J=11.2, 2.9 Hz, 1H), 6.40 (td, J=8.5, 2.9 Hz, 1H). ¹³C NMR (201 MHz, DMSO) δ 166.19, 162.45, 161.34 (d, J=238.8 Hz), 153.40, 149.53, 145.28 (d, J=12.21 Hz), 139.26, 129.29, 127.77 (d, J=10.69 Hz), 122.75, 119.67, 102.90 (d, J=22.12 Hz), 102.40 (d, J=25.17 Hz).

### Example 37

### N²-(2-Amino-4-fluorophenyl)-N⁵-((2S,3R)-3-hydroxy-4-((N-isobutyl-4-nitrophenyl)sulfonamido)-1-phenylbutan-2-yl)pyridine-2,5-dicarboxamide(35)

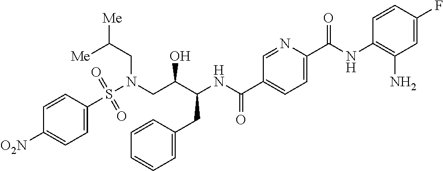 [SMILES: CC(C)CN(C[C@@H](O)[C@H](Cc1ccccc1)NC(=O)c1ccc(C(=O)Nc2ccc(F)cc2N)nc1)S(=O)(=O)c1ccc([N+](=O)[O-])cc1]

N-((2R,3S)-3-amino-2-hydroxy-4-phenylbutyl)-N-isobutyl-4-nitrobenzenesulfonamide (0.45 mmol, 0.9 eq.) was dissolved in acetonitrile (4 mL) and stirred in a 20 mL vial. 6-((2-amino-4-fluorophenyl)carbamoyl)nicotinic acid (34) (0.5 mmol, 1 eq.), EDC·HCl (0.5 mmol, 1 eq.), HOBt·H₂O (0.05 mmol, 0.1 eq.), DMAP (0.5 mmol, 1 eq.), and DIPEA (2.5 mmol, 5 eq.) were added in sequence to the reaction mixture and stirred at room temperature for 16-36 hours. Upon reaction completion, the reaction mixture was diluted with water and extracted with ethyl acetate. The organic layer was isolated and washed with 1 M HCl solution (2×15 mL), saturated NaHCO₃ (2×15 mL), and brine (2×15 mL), dried with sodium sulfate, and concentrated under reduced pressure. The crude product was purified via column chromatography (EtOAc/hexanes [10:90 to 70:30]) to afford pure compound 35. Yellow powder (0.115 g, 46.7% yield). ¹H NMR (800 MHz, DMSO) δ 10.03 (s, 1H), 8.93 (s, 1H), 8.67 (d, J=8.9 Hz, 1H), 8.34 (d, J=8.3 Hz, 2H), 8.28 (d, J=8.2 Hz, 1H), 8.19 (d, J=8.0 Hz, 1H), 8.04 (d, J=8.1 Hz, 2H), 7.31 (t, J=7.5 Hz, 1H), 7.27 (d, J=7.6 Hz, 2H), 7.23 (t, J=7.5 Hz, 2H), 7.13 (t, J=7.4 Hz, 1H), 6.59 (dd, J=11.1, 2.8 Hz, 1H), 6.41 (t, J=8.7 Hz, 1H), 5.26 (d, J=10.1 Hz, 3H), 4.11 (d, J=9.0 Hz, 1H), 3.77 (d, J=7.5 Hz, 1H), 3.47 (d, J=14.7 Hz, 1H), 3.21 (dd, J=13.6, 8.6 Hz, 1H), 3.16-3.08 (m, 2H), 2.94 (dd, J=13.6, 6.4 Hz, 1H), 2.75 (t, J=12.6 Hz, 1H), 2.02 (dt, J=14.2, 7.0 Hz, 1H), 0.87 (d, J=6.6 Hz, 3H), 0.81 (d, J=6.6 Hz, 3H). ¹³C NMR (201 MHz, DMSO) δ 164.43, 162.55, 161.32 (d, J=238.8 Hz), 152.18, 150.00, 147.64, 145.54, 145.28 (d, J=11.44 Hz), 139.74, 137.05, 132.59, 129.57, 129.04, 128.49, 127.79 (d, J=10.68 Hz), 126.39, 124.89, 122.39, 119.73, 102.92 (d, J=22.88 Hz), 102.40(d, J=25.18 Hz), 72.01, 56.21, 54.97, 52.11, 35.33, 26.38, 20.31, 20.22.

### Example 38

### N²-(2-amino-4-fluorophenyl)-N⁵-((2S,3R)-4-((4-amino-N-isobutylphenyl)sulfonamido)-3-hydroxy-1-phenylbutan-2-yl)pyridine-2,5-dicarboxamide (NB-5)

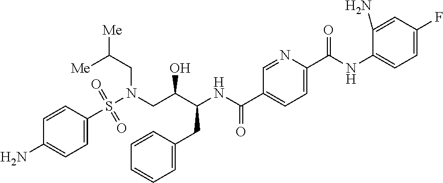 [SMILES: CC(C)CN(C[C@@H](O)[C@H](Cc1ccccc1)NC(=O)c1ccc(C(=O)Nc2ccc(F)cc2N)nc1)S(=O)(=O)c1ccc(N)cc1]

Compound 35 was dissolved in methanol along with PtO₂ (0.1 eq.) and potassium carbonate (0.1 eq.) in a round-bottom flask that was sealed. Air was purged from the reaction vessel using 2 hydrogen balloons, and a third hydrogen balloon was set up to stir overnight. Upon reaction completion, the mixture was filtered and purified via column chromatography (EtOAc/hexanes [40:60 to 70:30]) to afford pure compound NB-5. Yellow powder (0.055 g, 82.4%). ¹H NMR (800 MHz, DMSO) δ 10.04 (s, 1H), 8.97-8.95 (m, 1H), 8.70 (d, J=9.1 Hz, 1H), 8.29 (dd, J=8.2, 2.3 Hz, 1H), 8.20 (d, J=8.0 Hz, 1H), 7.32 (d, J=8.4 Hz, 2H), 7.31-7.28 (m, 3H), 7.22 (t, J=7.4 Hz, 2H), 7.13 (t, J=7.3 Hz, 1H), 6.59 (dd, J=11.1, 2.9 Hz, 1H), 6.53 (d, J=8.5 Hz, 2H), 6.41 (td, J=8.5, 2.9 Hz, 1H), 5.96 (s, 2H), 5.26 (s, 2H), 5.22 (d, J=6.7 Hz, 1H), 4.13 (d, J=8.9 Hz, 1H), 3.84 (d, J=7.9 Hz, 1H), 3.40 (s, 1H), 3.20 (d, J=12.7 Hz, 1H), 3.00 (dd, J=13.3, 8.7 Hz, 1H), 2.76 (t, J=12.7 Hz, 1H), 2.71 (dd, J=14.9, 8.1 Hz, 1H), 2.64 (dd, J=13.3, 6.1 Hz, 1H), 2.01 (dd, J=15.2, 8.1 Hz, 1H), 0.89 (d, J=6.5 Hz, 3H), 0.81 (d, J=6.6 Hz, 3H). ¹³C NMR (201 MHz, DMSO) δ 164.36, 162.61, 161.33 (d, J=239.57 Hz), 153.20, 152.12, 147.65, 145.31 (d, J=11.44 Hz), 140.03, 137.09, 132.77, 129.61, 129.45, 128.44, 127.84, 127.79, 126.30, 123.83, 122.35, 119.73, 113.09, 102.91 (d, J=22.89 Hz), 102.40 (d, J=25.18 Hz), 73.36, 57.94, 54.99, 53.54, 35.31, 26.81, 20.58, 20.56.

### Example 39

### methyl 6-(2-(tert-butoxycarbonyl)-2-propylhydrazine-1-carbonyl)nicotinate

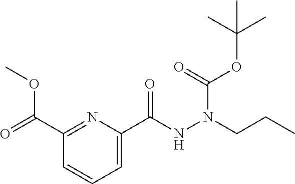 [SMILES: CCCN(NC(=O)c1cccc(C(=O)OC)n1)C(=O)OC(C)(C)C]

A vial was charged with 5-(methoxycarbonyl)picolinic acid (0.0906 g, 0.5 mmol) and HATU (0.2282 g, 1.2 eq.) and dissolved in DMF (4 mL). DIPEA was added dropwise and stirred for 15 minutes. After 15 minutes, tert-butyl 1-propylhydrazine-1-carboxylate (0.0875 mL, 0.9 eq.) was added to the reaction mixture and allowed to stir overnight. Upon reaction completion, the mixture was diluted with water and extracted into ethyl acetate. The organic layer was washed with 1M HCl (2×15 mL), sat. NaHCO₃ (2×15 mL), and brine (2×15 mL), dried with Na₂SO₄, and concentrated under reduced pressure. The crude mixture was purified via column chromatography (DCM/MeOH [100:0 to 97:3]). Clear oil (0.110 g, 65.2%). ¹H NMR (400 MHz, DMSO) δ 10.88 (d, J=20.4 Hz, 1H), 9.10 (d, J=1.8 Hz, 1H), 8.47 (dd, J=8.1, 2.1 Hz, 1H), 8.14 (d, J=8.1 Hz, 1H), 3.91 (s, 3H), 3.37 (t, J=7.2 Hz, 2H), 1.48 (d, J=7.2 Hz, 1H), 1.41 (s, 4H), 1.28 (s, 6H), 0.85 (s, 3H). ¹³C NMR (101 MHz, DMSO) δ 164.96, 162.63, 155.38, 152.77, 149.37, 139.00, 128.40, 122.90, 53.09, 37.03, 28.19, 21.06, 15.34, 11.54.

### Example 40

### 6-(2-(tert-butoxycarbonyl)-2-propylhydrazine-1-carbonyl)nicotinic acid

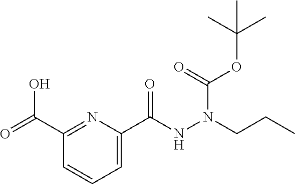 [SMILES: CCCN(NC(=O)c1cccc(C(=O)O)n1)C(=O)OC(C)(C)C]

Methyl 6-(2-(tert-butoxycarbonyl)-2-propylhydrazine-1-carbonyl)nicotinate was dissolved in 8 mL THF in a 20 mL vial and stirred. To the reaction mixture, 1.5 mL aq. LiOH solution was added dropwise and stirred overnight. Upon reaction conclusion, the mixture was diluted with water, neutralized with HCl, extracted with ethyl acetate, and dried with sodium sulfate. The dried organic layer was concentrated under reduced pressure to afford the pure product. White powder (0.1313 g, 86.5%). 1H NMR (400 MHz, DMSO) δ 10.77 (d, J=21.0 Hz, 1H), 9.07 (s, 1H), 8.39 (d, J=8.1 Hz, 1H), 8.05 (d, J=8.0 Hz, 1H), 3.36 (s, 2H), 1.47 (s, 2H), 1.35 (d, J=51.6 Hz, 9H), 0.85 (s, 3H). 13C NMR (101 MHz, DMSO) δ 166.96, 163.12, 157.72, 151.11, 149.71, 138.60, 135.09, 128.08, 122.40, 33.72, 30.07, 28.21, 20.67, 11.56.

### Example 41

### tert-butyl 2-(5-(((2S,3R)-3-hydroxy-4-((N-isobutyl-4-nitrophenyl)sulfonamido)-1-phenylbutan-2-yl)carbamoyl)picolinoyl)-1-propylhydrazine-1-carboxylate

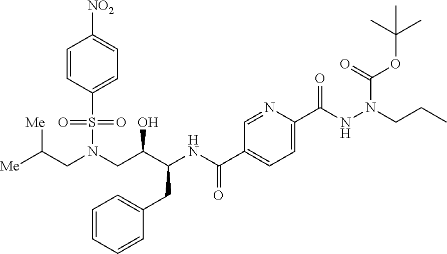 [SMILES: CCCN(NC(=O)c1ccc(C(=O)N[C@@H](Cc2ccccc2)[C@H](O)CN(CC(C)C)S(=O)(=O)c2ccc([N+](=O)[O-])cc2)cn1)C(=O)OC(C)(C)C]

N-((2R,3S)-3-amino-2-hydroxy-4-phenylbutyl)-N-isobutyl-4-nitrobenzenesulfonamide (0.1434 g, 0.9 eq.) was dissolved in acetonitrile (4 mL) and stirred in a 20 mL vial. 6-(2-(tert-butoxycarbonyl)-2-propylhydrazine-1-carbonyl)nicotinic acid (0.110 g, 1 eq.), EDC·HCl (0.0646 g, 1 eq.), HOBt. H₂O (0.0053 g, 0.1 eq.), DMAP (0.0415 g, 1 eq.), and DIPEA (0.2961 mL, 5 eq.) were added in sequence to the reaction mixture and stirred at room temperature for 16-36 hours. Upon reaction completion, the reaction mixture was diluted with water and extracted with ethyl acetate. The organic layer was isolated and washed with 1 M HCl solution (2×15 mL), saturated NaHCO₃ (2×15 mL), and brine (2×15 mL), dried with sodium sulfate, and concentrated under reduced pressure. The crude product was purified via column chromatography (EtOAc/hexanes [10:90 to 70:30]) to afford pure product. White powder (0.0567 g, 22.9%). ¹H NMR (800 MHz, DMSO) δ 10.81 (d, J=44.6 Hz, 1H), 8.86 (s, 1H), 8.65 (d, J=9.0 Hz, 1H), 8.33 (d, J=8.3 Hz, 2H), 8.24 (dd, J=8.1, 2.0 Hz, 1H), 8.10 (s, 2H), 8.04 (d, J=8.5 Hz, 2H), 7.26 (d, J=7.6 Hz, 2H), 7.22 (t, J=7.5 Hz, 2H), 7.13 (t, J=7.4 Hz, 1H), 5.24 (d, J=6.9 Hz, 1H), 4.10 (q, J=10.6 Hz, 1H), 3.75 (d, J=7.6 Hz, 1H), 3.46 (d, J=14.7 Hz, 1H), 3.39 (s, 2H), 3.20 (dd, J=13.6, 8.6 Hz, 1H), 3.12 (d, J=11.8 Hz, 2H), 2.93 (dd, J=13.6, 6.4 Hz, 1H), 2.74 (t, J=12.6 Hz, 1H), 2.00 (dt, J=14.0, 7.0 Hz, 1H), 1.50 (s, 3H), 1.44 (s, 3H), 1.30 (s, 5H), 0.87 (s, 3H), 0.86 (d, J=6.6 Hz, 3H), 0.80 (d, J=6.6 Hz, 3H). ¹³C NMR (201 MHz, DMSO) δ 164.46, 162.89, 154.99, 151.40, 150.00, 147.84, 145.56, 139.72, 136.97, 132.98, 129.56, 129.04, 128.49, 126.40, 124.87, 122.57, 79.90, 71.95, 56.17, 54.96, 52.08, 50.65, 35.28, 28.43, 28.27, 26.36, 20.29, 20.21, 11.68.

### Example 42

### N-((2S,3R)-3-hydroxy-4-((N-isobutyl-4-nitrophenyl)sulfonamido)-1-phenylbutan-2-yl)-6-(2-propylhydrazine-1-carbonyl)nicotinamide

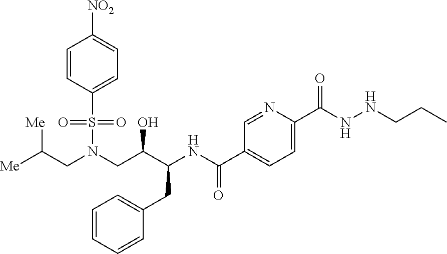 [SMILES: CCCNNC(=O)c1ccc(C(=O)N[C@@H](Cc2ccccc2)[C@H](O)CN(CC(C)C)S(=O)(=O)c2ccc([N+](=O)[O-])cc2)cn1]

tert-butyl 2-(5-(((2S,3R)-3-hydroxy-4-((N-isobutyl-4-nitrophenyl)sulfonamido)-1-phenylbutan-2-yl)carbamoyl)picolinoyl)-1-propylhydrazine-1-carboxylate (0.045 g) was dissolved in 5 mL of DCM. Anhydrous 4 N HCl in dioxane was added dropwise (2 mL) to the reaction at room temperature and stirred for 8-16 hours. Upon reaction completion, saturated sodium bicarbonate solution was added until the addition did not result in bubbles. The product was then extracted into DCM, washed with bring (2×15 mL), dried with sodium sulfate, and concentrated via reduced pressure to afford pure product. Off-white powder (0.0387 g, 99.7%). ¹H NMR (800 MHz, DMSO) δ 10.25 (s, 1H), 8.83 (d, J=2.0 Hz, 1H), 8.62 (d, J=9.0 Hz, 1H), 8.33 (d, J=8.7 Hz, 2H), 8.21 (dd, J=8.1, 2.2 Hz, 1H), 8.06 (d, J=8.1 Hz, 1H), 8.03 (d, J=8.7 Hz, 2H), 7.26 (d, J=7.6 Hz, 2H), 7.22 (t, J=7.6 Hz, 2H), 7.12 (t, J=7.3 Hz, 1H), 5.23 (d, J=6.9 Hz, 1H), 4.09 (q, J=10.7 Hz, 1H), 3.74 (q, J=8.9 Hz, 1H), 3.47-3.43 (m, 1H), 3.19 (dd, J=13.6, 8.6 Hz, 1H), 3.14-3.07 (m, 2H), 2.93 (dd, J=13.5, 6.4 Hz, 1H), 2.79 (t, J=7.1 Hz, 2H), 2.76-2.71 (m, 1H), 2.00 (dp, J=13.7, 6.8 Hz, 1H), 1.46 (hept, J=7.2 Hz, 2H), 0.90 (t, J=7.4 Hz, 3H), 0.86 (d, J=6.5 Hz, 3H), 0.80 (d, J=6.6 Hz, 3H). ¹³C NMR (201 MHz, DMSO) δ 164.50, 162.01, 152.00, 150.00, 147.73, 145.54, 139.73, 136.88, 132.37, 129.55, 129.03, 128.48, 126.38, 124.87, 122.00, 71.96, 56.18, 54.93, 53.28, 52.08, 35.30, 26.36, 21.15, 20.30, 20.21, 12.05.

### Example 43

### N-((2S,3R)-4-((4-amino-N-isobutylphenyl)sulfonamido)-3-hydroxy-1-phenylbutan-2-yl)-6-(2-propylhydrazine-1-carbonyl)nicotinamide (NB-7)

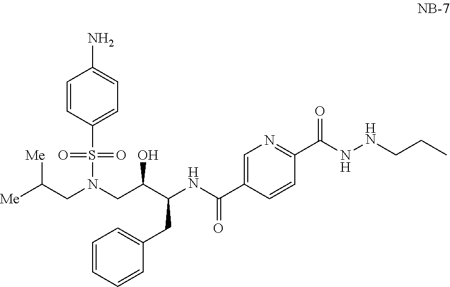 [SMILES: CCCNNC(=O)c1ccc(C(=O)N[C@@H](Cc2ccccc2)[C@H](O)CN(CC(C)C)S(=O)(=O)c2ccc(N)cc2)cn1]

N-((2S,3R)-3-hydroxy-4-((N-isobutyl-4-nitrophenyl)sulfonamido)-1-phenylbutan-2-yl)-6-(2-propylhydrazine-1-carbonyl)nicotinamide (0.035 g) was dissolved in methanol along with PtO₂ (0.1 eq.) and potassium carbonate (0.1 eq.) in a round bottom flask that was then sealed. Air was purged from the reaction vessel using 2 hydrogen balloons, and a third hydrogen balloon was set up to stir overnight. Upon reaction completion, the mixture was filtered and purified via column chromatography (EtOAc/hexanes [40:60 to 70:30]) to afford pure product. Off-white powder (0.0213 g, 63.9%). ¹H NMR (800 MHz, DMSO) δ 10.26 (s, 1H), 8.86 (s, 1H), 8.66 (d, J=9.0 Hz, 1H), 8.22 (dd, J=8.0, 2.1 Hz, 1H), 8.07 (d, J=8.1 Hz, 1H), 7.31 (d, J=8.4 Hz, 1H), 7.27 (q, J=8.6 Hz, 3H), 7.21 (t, J=7.6 Hz, 3H), 7.12 (t, J=7.3 Hz, 1H), 6.55-6.48 (m, 2H), 5.96 (s, 2H), 5.20 (d, J=6.7 Hz, 1H), 5.03 (s, 1H), 4.11 (q, J=10.2 Hz, 1H), 3.81 (s, 1H), 3.25 (s, 1H), 3.18 (d, J=12.7 Hz, 1H), 2.98 (dd, J=13.4, 8.7 Hz, 1H), 2.79 (t, J=7.2 Hz, 2H), 2.75 (d, J=12.2 Hz, 1H), 2.69 (dd, J=14.8, 8.1 Hz, 1H), 2.63 (dd, J=13.3, 6.1 Hz, 1H), 1.99 (q, J=7.0 Hz, 1H), 1.46 (hept, J=7.3 Hz, 2H), 1.24 (s, 1H), 0.90 (t, J=7.4 Hz, 3H), 0.87 (d, J=6.7 Hz, 3H), 0.79 (d, J=6.7 Hz, 3H). ¹³C NMR (201 MHz, DMSO) δ 164.43, 162.07, 153.20, 151.93, 147.76, 140.04, 136.90, 132.54, 129.60, 129.44, 128.43, 126.29, 123.83, 121.96, 113.08, 73.30, 57.93, 55.79, 54.94, 53.50, 35.26, 26.79, 21.16, 20.55, 12.05. HRMS (ESI) m/z: calcd for C₃₀H₄₀N₆O₅S [M+H]⁺, 597.2854, found 597.2863.

### Example 44

### tert-butyl 2-(5-methoxy-5-oxopentanoyl)-1-propylhydrazine-1-carboxylate

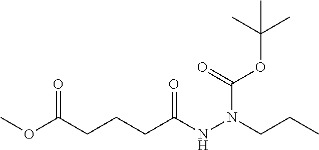 [SMILES: CCCN(NC(=O)CCCC(=O)OC)C(=O)OC(C)(C)C]

5-methoxy-5-oxopentanoic acid (0.0628 mL, 0.5 mmol) was dissolved in 3 mL DMF and stirred. To the reaction mixture, triethylamine (1.2 eq.) was added at room temperature, followed by TBTU (1.2 eq.). The reaction was stirred at room temperature for 15 minutes. After 15 minutes, tert-butyl 1-propylhydrazine-1-carboxylate (0.0832 mL, 0.9 eq.) was added to the reaction and stirred for 16 hours. Upon reaction completion, the mixture was diluted with water (25 mL) and extracted into ethyl acetate (3×20 mL). The organic layer was washed with saturated sodium bicarbonate (2×15 mL) and brine (2×15 mL), dried over sodium sulfate, concentrated under reduced pressure, and purified via column chromatography (ethyl acetate/hexanes [10:90 to 50:50]) to afford pure product. Clear oil (0.1104 g, 73.0%). ¹H NMR (400 MHz, DMSO) δ 9.87 (s, 1H), 3.56 (s, 3H), 3.23 (s, 2H), 2.31 (t, J=7.4 Hz, 2H), 2.08 (t, J=7.3 Hz, 2H), 1.75 (q, J=7.5 Hz, 2H), 1.35 (d, J=19.4 Hz, 11H), 0.81 (t, J=7.4 Hz, 3H). ¹³C NMR (101 MHz, DMSO) δ 173.31, 163.57, 157.07, 51.61, 50.30, 41.75, 32.82, 32.43, 28.22, 22.13, 20.67, 11.44.

### Example 45

### N-((2S,3R)-3-hydroxy-4-((N-isobutyl-4-nitrophenyl)sulfonamido)-1-phenylbutan-2-yl)-5-oxo-5-(2-propylhydrazineyl)pentanamide

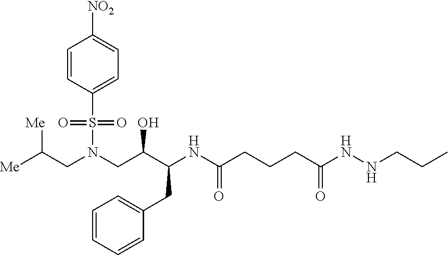 [SMILES: CCCNNC(=O)CCCC(=O)N[C@@H](Cc1ccccc1)[C@H](O)CN(CC(C)C)S(=O)(=O)c1ccc([N+](=O)[O-])cc1]

tert-butyl 2-(5-(((2S,3R)-3-hydroxy-4-((N-isobutyl-4-nitrophenyl)sulfonamido)-1-phenylbutan-2-yl)amino)-5-oxopentanoyl)-1-propylhydrazine-1-carboxylate (0.070 g) was dissolved in 5 mL of DCM. Anhydrous 4 N HCl in dioxane was added dropwise (2 mL) to the reaction at room temperature and stirred for 8-16 hours. Upon reaction completion, saturated sodium bicarbonate solution was added until the addition did not result in bubbles. The product was then extracted into DCM, washed with bring (2×15 mL), dried with sodium sulfate, and concentrated via reduced pressure to afford pure product. Off-white powder (0.0448 g, 74.8%). ¹H NMR (800 MHz, DMSO) δ 9.17 (s, 1H), 8.39 (d, J=8.4 Hz, 2H), 8.05 (d, J=8.5 Hz, 2H), 7.74 (d, J=9.0 Hz, 1H), 7.22 (t, J=7.5 Hz, 2H), 7.17 (d, J=7.6 Hz, 2H), 7.14 (t, J=7.4 Hz, 1H), 5.07 (d, J=7.0 Hz, 1H), 3.79 (d, J=9.1 Hz, 1H), 3.52 (d, J=7.9 Hz, 1H), 3.34 (s, 1H), 3.16 (dd, J=13.7, 8.6 Hz, 1H), 3.04-2.96 (m, 2H), 2.91 (dd, J=13.7, 6.5 Hz, 1H), 2.60 (t, J=7.2 Hz, 2H), 2.48 (d, J=12.3 Hz, 1H), 1.97 (dd, J=14.9, 8.0 Hz, 4H), 1.89 (dp, J=31.9, 7.8 Hz, 4H), 1.55 (p, J=7.6 Hz, 2H), 1.38 (p, J=7.4 Hz, 2H), 0.86 (dd, J=10.0, 6.8 Hz, 6H), 0.81 (d, J=6.7 Hz, 3H). ¹³C NMR (201 MHz, DMSO) δ 171.90, 171.01, 149.99, 145.73, 139.74, 129.53, 129.03, 128.37, 126.24, 124.90, 71.89, 55.94, 53.77, 53.51, 51.81, 35.63, 35.39, 33.45, 26.27, 22.14, 21.20, 20.29, 20.16, 12.04.

### Example 46

### N-((2S,3R)-4-((4-amino-N-isobutylphenyl)sulfonamido)-3-hydroxy-1-phenylbutan-2-yl)-5-oxo-5-(2-propylhydrazineyl)pentanamide (NB-10)

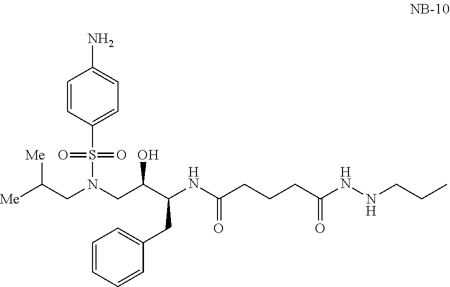 [SMILES: CCCNNC(=O)CCCC(=O)N[C@@H](Cc1ccccc1)[C@H](O)CN(CC(C)C)S(=O)(=O)c1ccc(N)cc1]

N-((2S,3R)-3-hydroxy-4-((N-isobutyl-4-nitrophenyl)sulfonamido)-1-phenylbutan-2-yl)-5-oxo-5-(2-propylhydrazineyl)pentanamide (0.038 g) was dissolved in methanol along with PtO₂ (0.1 eq.) and potassium carbonate (0.1 eq.) in a round bottom flask that was then sealed. Air was purged from the reaction vessel using 2 hydrogen balloons, and a third hydrogen balloon was set up to stir overnight. Upon reaction completion, the mixture was filtered and purified via column chromatography (EtOAc/hexanes [40:60 to 70:30]) to afford pure product. Off-white powder (0.019 g, 52.7%). ¹H NMR (800 MHz, DMSO) δ 9.15 (s, 1H), 7.73 (d, J=9.1 Hz, 1H), 7.39 (d, J=8.6 Hz, 2H), 7.21 (dt, J=14.4, 7.6 Hz, 5H), 7.13 (t, J=7.1 Hz, 1H), 6.61 (d, J=8.6 Hz, 2H), 5.97 (s, 2H), 5.00 (d, J=6.5 Hz, 1H), 3.86-3.80 (m, 1H), 3.65-3.59 (m, 1H), 3.29-3.24 (m, 1H), 3.03 (dd, J=13.8, 3.3 Hz, 1H), 2.94 (dd, J=13.5, 8.5 Hz, 1H), 2.64 (dd, J=15.1, 8.4 Hz, 2H), 2.61 (t, J=7.2 Hz, 2H), 1.94 (ddq, J=29.3, 14.5, 7.1 Hz, 4H), 1.87 (h, J=7.0 Hz, 2H), 1.59-1.51 (m, 2H), 1.38 (h, J=7.3 Hz, 2H), 0.87 (t, J=7.5 Hz, 3H), 0.85 (d, J=6.6 Hz, 3H), 0.79 (d, J=6.7 Hz, 3H). ¹³C NMR (201 MHz, DMSO) δ 171.71, 171.01, 153.19, 140.05, 129.61, 129.50, 128.31, 126.14, 124.05, 113.16, 73.04, 57.72, 53.76, 53.52, 53.24, 40.47, 35.39, 33.51, 26.78, 22.12, 21.22, 20.55, 12.06. HRMS (ESI) m/z: calcd for C₂₈H₄₃N₅O₅S [M+H]⁺, 562.3058, found 562.3065.

### Example 47

### tert-butyl 2-(6-methoxy-6-oxohexanoyl)-1-propylhydrazine-1-carboxylate

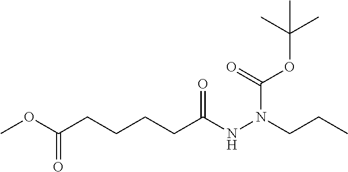 [SMILES: CCCN(NC(=O)CCCCC(=O)OC)C(=O)OC(C)(C)C]

6-methoxy-6-oxohexanoic acid (0.0711 mL, 0.5 mmol) was dissolved in 3 mL DMF and stirred. To the reaction mixture, triethylamine (1.2 eq.) was added at room temperature, followed by TBTU (1.2 eq.). The reaction was stirred at room temperature for 15 minutes. After 15 minutes, tert-butyl 1-propylhydrazine-1-carboxylate (0.0832 mL, 0.9 eq.) was added to the reaction and stirred for 16 hours. Upon reaction completion, the mixture was diluted with water (25 mL) and extracted into ethyl acetate (3×20 mL). The organic layer was washed with saturated sodium bicarbonate (2×15 mL) and brine (2×15 mL), dried over sodium sulfate, concentrated under reduced pressure, and purified via column chromatography (ethyl acetate/hexanes [10:90 to 50:50]) to afford pure product. Clear oil (0.1122 g, 70.9%). ¹H NMR (400 MHz, DMSO) δ 9.84 (s, 1H), 3.55 (s, 3H), 3.23 (s, 2H), 2.32-2.23 (m, 2H), 2.05 (d, J=6.7 Hz, 1H), 1.50 (s, 4H), 1.44-1.29 (m, 11H), 0.81 (t, J=7.4 Hz, 3H). ¹³C NMR (101 MHz, DMSO) δ 184.29, 173.54, 172.32, 51.54, 50.28, 48.29, 33.32, 33.06, 31.93, 28.21, 24.76, 24.30, 11.44.

### Example 48

### tert-butyl 2-(6-(((2S,3R)-3-hydroxy-4-((N-isobutyl-4-nitrophenyl)sulfonamido)-1-phenylbutan-2-yl)amino)-6-oxohexanoyl)-1-propylhydrazine-1-carboxylate

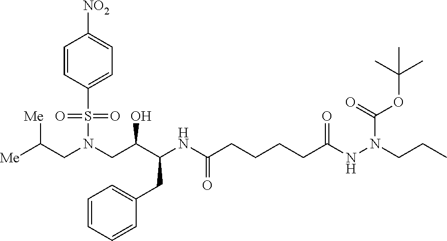 [SMILES: CCCN(NC(=O)CCCCC(=O)N[C@@H](Cc1ccccc1)[C@H](O)CN(CC(C)C)S(=O)(=O)c1ccc([N+](=O)[O-])cc1)C(=O)OC(C)(C)C]

6-(2-(tert-butoxycarbonyl)-2-propylhydrazineyl)-6-oxohexanoic acid (0.050 g, 1 eq.) was dissolved in 3 mL DMF and stirred. To the reaction mixture, triethylamine (1.2 eq.) was added at room temperature, followed by TBTU (1.2 eq.). The reaction was stirred at room temperature for 15 minutes. After 15 minutes, N-((2R,3S)-3-amino-2-hydroxy-4-phenylbutyl)-N-isobutyl-4-nitrobenzenesulfonamide (0.0697 g, 0.9 eq.) was added to the reaction and stirred for 16 hours. Upon reaction completion, the mixture was diluted with water (25 mL) and extracted into ethyl acetate (3×20 mL). The organic layer was washed with saturated sodium bicarbonate (2×15 mL) and brine (2×15 mL), dried over sodium sulfate, concentrated under reduced pressure, and purified via column chromatography (ethyl acetate/hexanes [40:60 to 70:30]) to afford pure product. Off-white powder (0.0684 g, 58.6%). ¹H NMR (800 MHz, DMSO) δ 9.81 (d, J=21.3 Hz, 1H), 8.39 (d, J=8.5 Hz, 2H), 8.06 (d, J=8.5 Hz, 2H), 7.74 (d, J=9.1 Hz, 1H), 7.22 (d, J=7.5 Hz, 1H), 7.18 (d, J=7.6 Hz, 2H), 7.13 (t, J=7.5 Hz, 1H), 5.06 (d, J=7.0 Hz, 1H), 3.80 (d, J=8.9 Hz, 1H), 3.53 (d, J=8.0 Hz, 1H), 3.36 (d, J=14.8 Hz, 1H), 3.26 (s, 2H), 3.16 (dd, J=13.7, 8.6 Hz, 1H), 3.01 (dd, J=33.3, 11.5 Hz, 2H), 2.91 (dd, J=13.7, 6.5 Hz, 1H), 2.49 (d, J=12.7 Hz, 1H), 1.99 (dtd, J=31.6, 15.2, 7.7 Hz, 5H), 1.59 (q, J=7.7 Hz, 2H), 1.40 (s, 6H), 1.32 (s, 5H), 1.21 (d, J=46.3 Hz, 1H), 0.87-0.82 (m, 6H), 0.81 (d, J=6.7 Hz, 3H). ¹³C NMR (201 MHz, DMSO) δ 171.79, 171.02, 155.30, 149.99, 145.77, 139.75, 129.53, 129.05, 128.37, 126.22, 124.89, 79.78, 71.86, 55.90, 53.76, 51.79, 50.39, 35.61, 35.40, 33.09, 28.32, 26.27, 21.94, 20.71, 20.30, 20.16, 11.57.

### Example 49

### N-((2S,3R)-3-hydroxy-4-((N-isobutyl-4-nitrophenyl)sulfonamido)-1-phenylbutan-2-yl)-6-oxo-6-(2-propylhydrazineyl)hexanamide

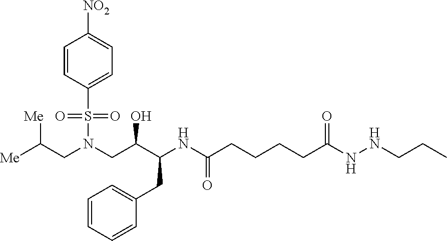 [SMILES: CCCNNC(=O)CCCCC(=O)N[C@@H](Cc1ccccc1)[C@H](O)CN(CC(C)C)S(=O)(=O)c1ccc([N+](=O)[O-])cc1]

tert-butyl 2-(6-(((2S,3R)-3-hydroxy-4-((N-isobutyl-4-nitrophenyl)sulfonamido)-1-phenylbutan-2-yl)amino)-6-oxohexanoyl)-1-propylhydrazine-1-carboxylate (0.055 g) was dissolved in 5 mL of DCM. Anhydrous 4 N HCl in dioxane was added dropwise (2 mL) to the reaction at room temperature and stirred for 8-16 hours. Upon reaction completion, saturated sodium bicarbonate solution was added until the addition did not result in bubbles. The product was then extracted into DCM, washed with bring (2×15 mL), dried with sodium sulfate, and concentrated via reduced pressure to afford pure product. Off-white powder (0.0381 g, 80.7%).

¹H NMR (800 MHz, DMSO) δ 9.18 (s, 1H), 8.39 (d, J=8.4 Hz, 2H), 8.05 (d, J=8.5 Hz, 2H), 7.69 (d, J=9.1 Hz, 1H), 7.22 (t, J=7.5 Hz, 2H), 7.17 (d, J=7.6 Hz, 2H), 7.14 (t, J=7.4 Hz, 1H), 5.05 (d, J=6.9 Hz, 1H), 3.80 (d, J=9.1 Hz, 1H), 3.52 (d, J=7.5 Hz, 1H), 3.36 (s, OH), 3.16 (dd, J=13.7, 8.5 Hz, 1H), 3.02 (dd, J=15.0, 8.8 Hz, 1H), 2.98 (d, J=12.3 Hz, 1H), 2.92 (dd, J=13.7, 6.4 Hz, 1H), 2.60 (t, J=7.2 Hz, 2H), 2.47 (d, J=12.3 Hz, 1H), 1.94 (dq, J=28.7, 6.8 Hz, 6H), 1.38 (h, J=7.2 Hz, 2H), 1.30 (s, 6H), 0.86 (dd, J=9.9, 6.8 Hz, 6H), 0.81 (d, J=6.7 Hz, 3H). ¹³C NMR (201 MHz, DMSO) δ 172.11, 171.16, 149.99, 145.78, 139.74, 129.53, 129.04, 128.36, 126.23, 124.90, 71.86, 55.86, 53.73, 53.53, 51.76, 35.68, 35.60, 33.71, 26.27, 25.27, 25.19, 21.22, 20.30, 20.16, 12.05.

### Example 50

### N-((2S,3R)-4-((4-amino-N-isobutylphenyl)sulfonamido)-3-hydroxy-1-phenylbutan-2-yl)-6-oxo-6-(2-propylhydrazineyl)hexanamide (NB-11)

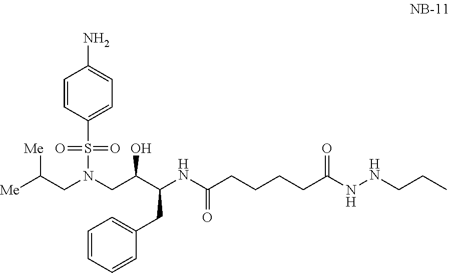 [SMILES: CCCNNC(=O)CCCCC(=O)N[C@@H](Cc1ccccc1)[C@H](O)CN(CC(C)C)S(=O)(=O)c1ccc(N)cc1]

N-((2S,3R)-3-hydroxy-4-((N-isobutyl-4-nitrophenyl)sulfonamido)-1-phenylbutan-2-yl)-6-oxo-6-(2-propylhydrazineyl)hexanamide (0.031 g) was dissolved in methanol along with PtO₂ (0.1 eq.) and potassium carbonate (0.1 eq.) in a round bottom flask that was then sealed. Air was purged from the reaction vessel using 2 hydrogen balloons, and a third hydrogen balloon was set up to stir overnight. Upon reaction completion, the mixture was filtered and purified via column chromatography (EtOAc/hexanes [40:60 to 70:30]) to afford pure product. Off-white powder (0.0219 g, 74.3%). ¹H NMR (800 MHz, DMSO) δ 9.18 (s, 1H), 7.69 (d, J=9.1 Hz, 1H), 7.39 (d, J=8.7 Hz, 2H), 7.21 (dt, J=15.3, 7.6 Hz, 5H), 7.13 (t, J=7.1 Hz, 1H), 6.61 (d, J=8.7 Hz, 2H), 5.98 (s, 2H), 5.00 (d, J=6.5 Hz, 1H), 3.87-3.81 (m, 1H), 3.64-3.59 (m, 1H), 3.26 (dd, J=14.7, 3.7 Hz, 1H), 3.03 (dd, J=13.9, 3.3 Hz, 1H), 2.94 (dd, J=13.4, 8.5 Hz, 1H), 2.66-2.62 (m, 2H), 2.61 (t, J=7.0 Hz, 2H), 2.00-1.88 (m, 8H), 1.41-1.34 (m, 2H), 1.33-1.23 (m, 2H), 0.88-0.84 (m, 6H), 0.79 (d, J=6.7 Hz, 3H). ¹³C NMR (201 MHz, DMSO) δ 171.95, 171.20, 153.19, 140.03, 129.61, 129.50, 128.30, 126.13, 124.01, 113.14, 73.06, 57.74, 53.72, 53.54, 53.27, 40.47, 35.70, 35.44, 33.74, 26.80, 25.27, 21.23, 20.55, 12.06. HRMS (ESI) m/z: calcd for C₂₉H₄₅N₅O₅S [M+H]⁺, 576.3214, found 576.3221.

### Enumerated Embodiments

The following list of enumerated embodiments presents claims with multiply dependent claims depending from multiply dependent claims for presentation in those jurisdictions where such dependencies are allowed as well as additional claims, which may be pursued during the examination of the application or a divisional thereof.
EE1. A compound of formula (I),

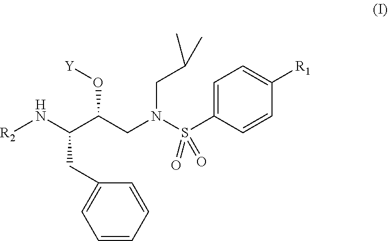

wherein Y is H, deuterium, or PO₃H;
R₁ is NH₂ or a group selected from:

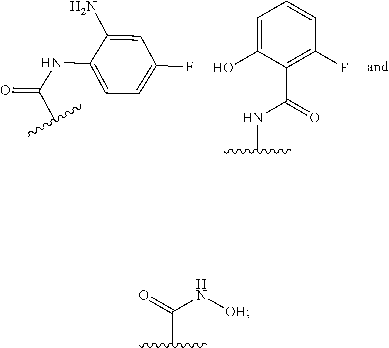

and
R₂ is G

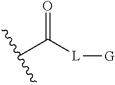

wherein L is O, C₁-C₅ alkyl, C₆-C₁₂ aryl, or C₅-C₁₂ heteroaryl; and
G is selected from the group consisting of (i), (ii), (iii), (iv), and (v):

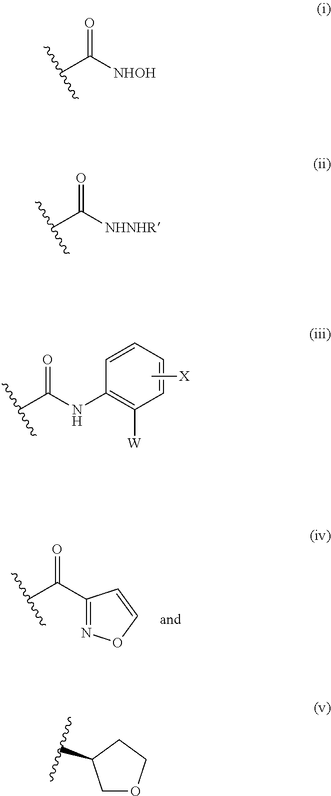

wherein R′ is C₁-C₅ alkyl;
X is H or halogen; and
W is OH, NH₂, or SCH₃;
with the proviso that when G is (v), R₁ is not NH₂,
or a pharmaceutically acceptable salt, hydrate, tautomer, or optical isomer thereof.
EE2. The compound of EE1, wherein R₁ is the group selected from:

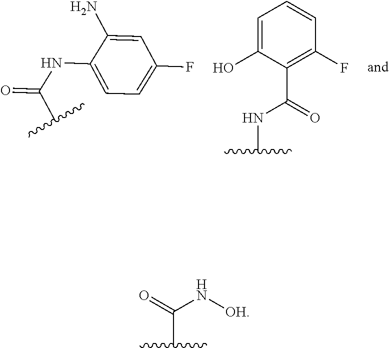

EE3. The compound of EE1, wherein R₁ is NH₂;
and R₂ is

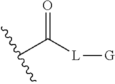

wherein L is o, C₁-C₅ alkyl, C₆-C₁₂ aryl, or C₅-C₁₂ heteroaryl; and
G is selected from the group consisting of (i), (ii), (iii), and (iv):

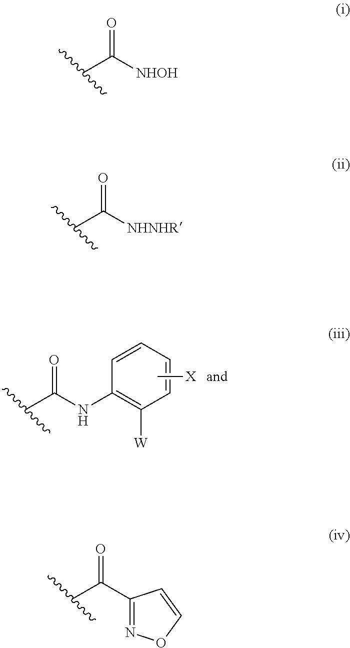

wherein R′ is C₁-C₅ alkyl;
X is H or halogen; and
W is OH, NH₂, or SCH₃.
EE4. The compound of EE1, wherein R₂ is

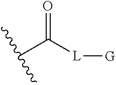

wherein L is O; and
G is

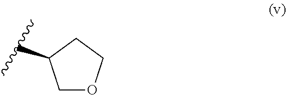 [SMILES: *[C@H]1CCOC1]

with the proviso that when G is (v), R₁ is not NH₂.
EE5. The compound of EE4, wherein R₁ is selected from:

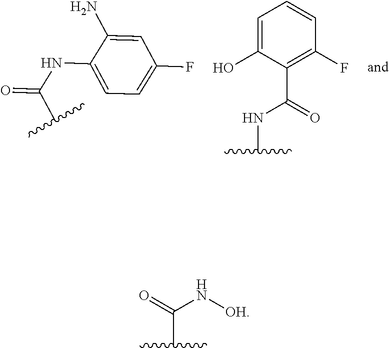

EE6. The compound of EE1, wherein R₂ is

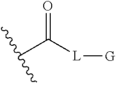

wherein L is C₆-C₁₂ aryl or C₅-C₁₂ heteroaryl;
and G is selected from the group consisting of (i), (ii), and (iii):

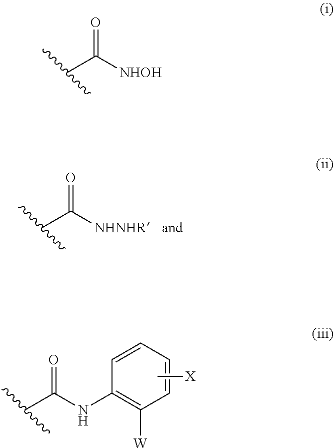

wherein R′ is C₁-C₅ alkyl;
X is H or halogen; and
W is NH₂.
EE7. The compound of EE6, wherein L is phenyl or pyridyl.
EE8. The compound of EE1, wherein the compound of formula (I) is:

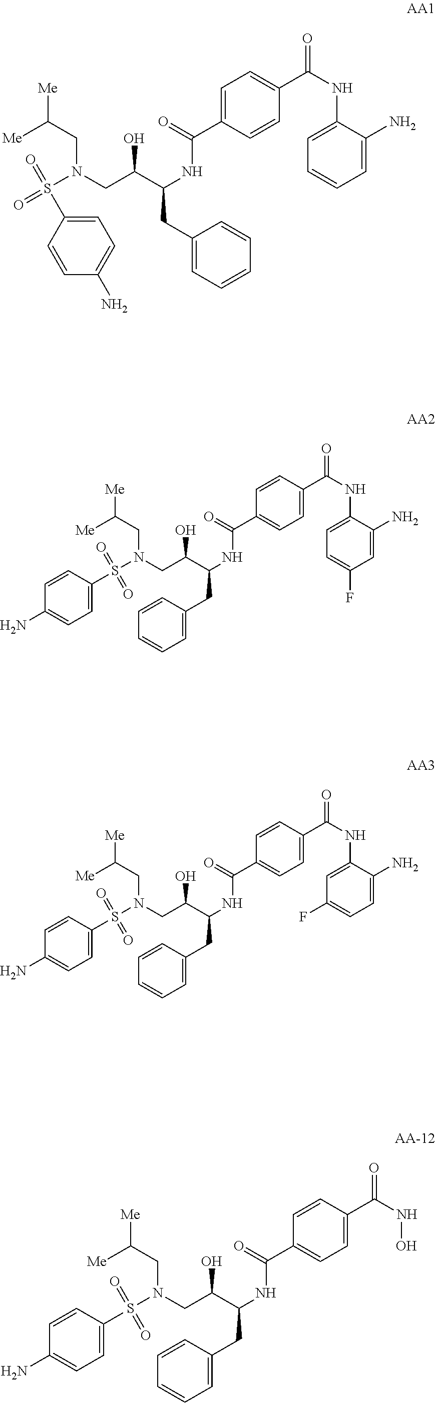 [SMILES: CC(C)CN(C[C@@H](O)[C@H](Cc1ccccc1)NC(=O)c1ccc(C(=O)Nc2ccccc2N)cc1)S(=O)(=O)c1ccc(N)cc1 ; CC(C)CN(C[C@@H](O)[C@H](Cc1ccccc1)NC(=O)c1ccc(C(=O)Nc2ccc(F)cc2N)cc1)S(=O)(=O)c1ccc(N)cc1 ; CC(C)CN(C[C@@H](O)[C@H](Cc1ccccc1)NC(=O)c1ccc(C(=O)Nc2cc(F)ccc2N)cc1)S(=O)(=O)c1ccc(N)cc1 ; CC(C)CN(C[C@@H](O)[C@H](Cc1ccccc1)NC(=O)c1ccc(C(=O)NO)cc1)S(=O)(=O)c1ccc(N)cc1]

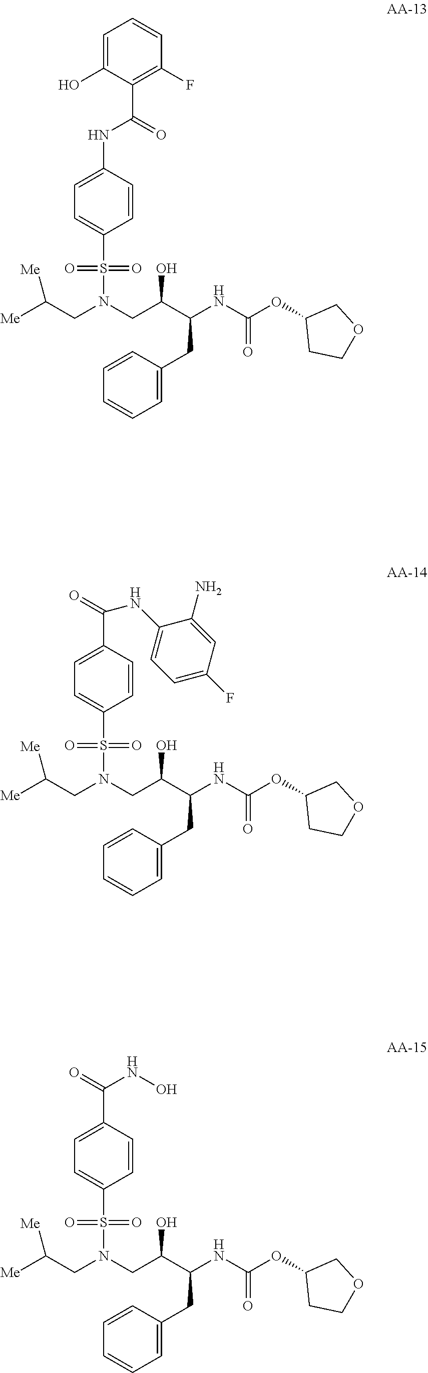 [SMILES: CC(C)CN(C[C@@H](O)[C@H](Cc1ccccc1)NC(=O)O[C@H]1CCOC1)S(=O)(=O)c1ccc(NC(=O)c2c(O)cccc2F)cc1 ; CC(C)CN(C[C@@H](O)[C@H](Cc1ccccc1)NC(=O)O[C@H]1CCOC1)S(=O)(=O)c1ccc(C(=O)Nc2ccc(F)cc2N)cc1 ; CC(C)CN(C[C@@H](O)[C@H](Cc1ccccc1)NC(=O)O[C@H]1CCOC1)S(=O)(=O)c1ccc(C(=O)NO)cc1]

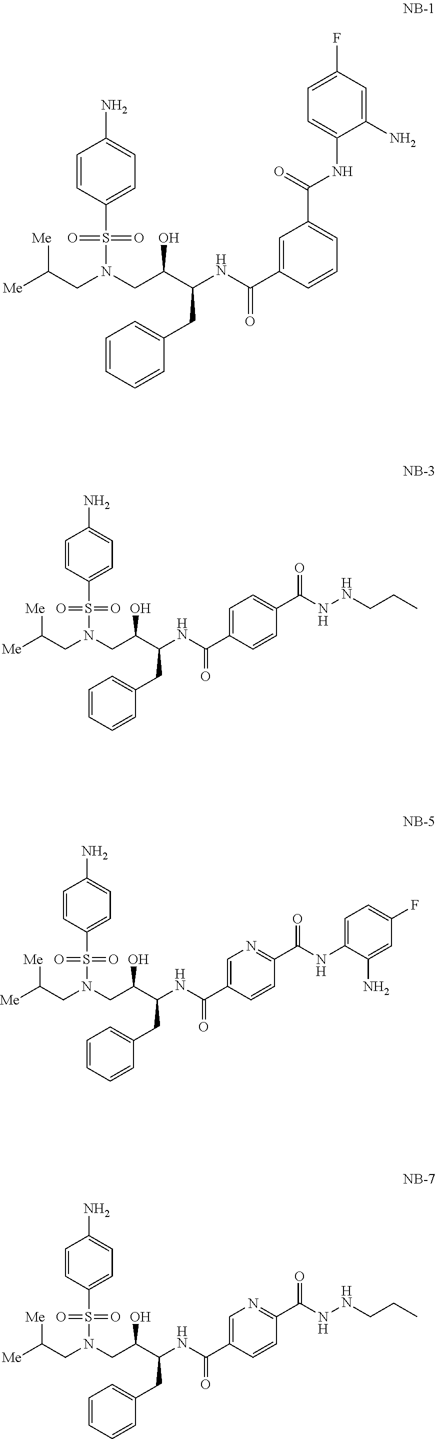 [SMILES: CC(C)CN(C[C@@H](O)[C@H](Cc1ccccc1)NC(=O)c1cccc(C(=O)Nc2ccc(F)cc2N)c1)S(=O)(=O)c1ccc(N)cc1 ; CCCNNC(=O)c1ccc(C(=O)N[C@@H](Cc2ccccc2)[C@H](O)CN(CC(C)C)S(=O)(=O)c2ccc(N)cc2)cc1 ; CC(C)CN(C[C@@H](O)[C@H](Cc1ccccc1)NC(=O)c1ccc(C(=O)Nc2ccc(F)cc2N)nc1)S(=O)(=O)c1ccc(N)cc1 ; CCCNNC(=O)c1ccc(C(=O)N[C@@H](Cc2ccccc2)[C@H](O)CN(CC(C)C)S(=O)(=O)c2ccc(N)cc2)cn1]

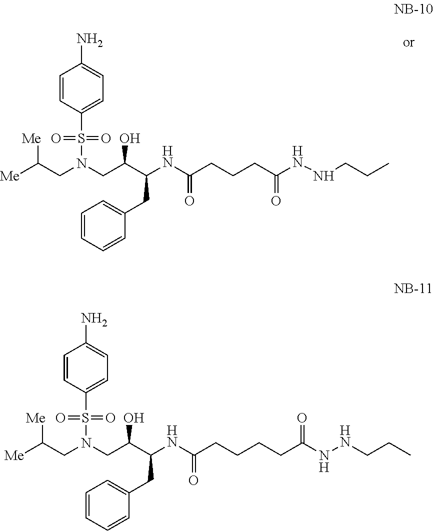 [SMILES: CCCNNC(=O)CCCC(=O)N[C@@H](Cc1ccccc1)[C@H](O)CN(CC(C)C)S(=O)(=O)c1ccc(N)cc1 ; CCCNNC(=O)CCCCC(=O)N[C@@H](Cc1ccccc1)[C@H](O)CN(CC(C)C)S(=O)(=O)c1ccc(N)cc1]

or a pharmaceutically acceptable salt, hydrate, tautomer, or optical isomer thereof.
EE9. The compound of EE1, wherein the compound of formula (I) is a compound of formula (IA)

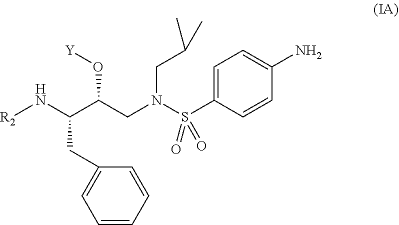

wherein
Y is H, deuterium, or PO₃H; and
R is selected from the group consisting of (a), (b), (c) and (d):

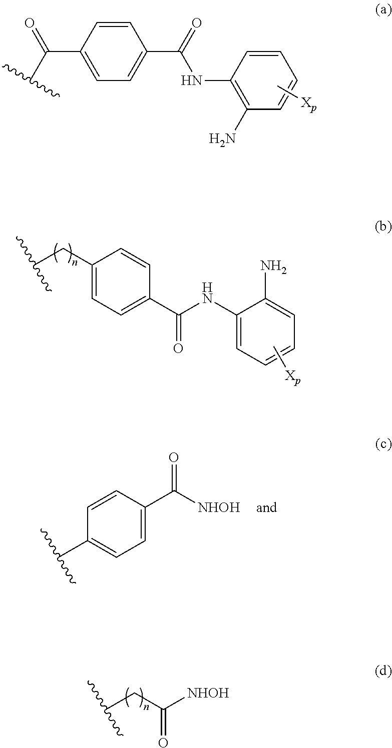

wherein Xₚ is halogen, wherein p is an integer from 1 to 4; and
n is an integer from 0 to 6;
or a pharmaceutically acceptable salt, hydrate, tautomer, or optical isomer thereof.
EE10. The compound of EE9, wherein X is fluorine.
EE11. The compound of EE9, wherein R is:

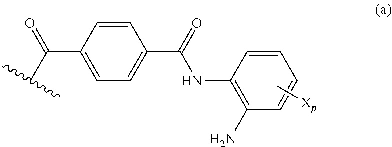

wherein Xₚ is as defined above.
EE12. The compound of EE9, wherein R is:

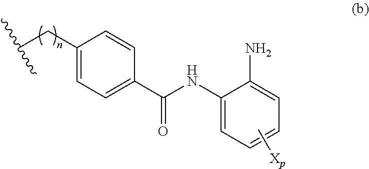

wherein Xₚ and n are as defined above.
EE13. The compound of EE9, wherein R is:

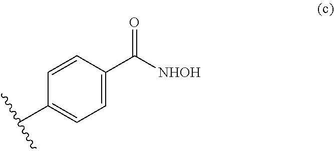

EE14. The compound of EE9, wherein the compound of formula (IA) is:

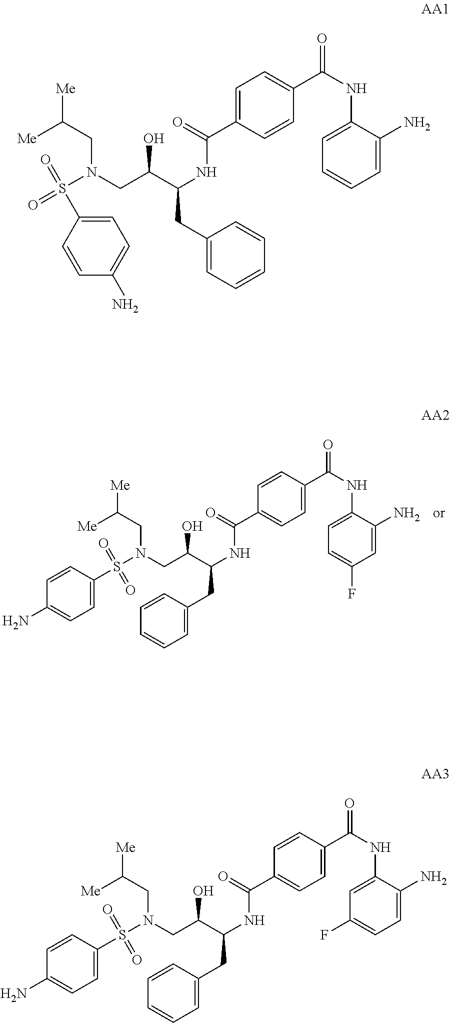 [SMILES: CC(C)CN(C[C@@H](O)[C@H](Cc1ccccc1)NC(=O)c1ccc(C(=O)Nc2ccccc2N)cc1)S(=O)(=O)c1ccc(N)cc1 ; CC(C)CN(C[C@@H](O)[C@H](Cc1ccccc1)NC(=O)c1ccc(C(=O)Nc2ccc(F)cc2N)cc1)S(=O)(=O)c1ccc(N)cc1 ; CC(C)CN(C[C@@H](O)[C@H](Cc1ccccc1)NC(=O)c1ccc(C(=O)Nc2cc(F)ccc2N)cc1)S(=O)(=O)c1ccc(N)cc1]

or a pharmaceutically acceptable salt, hydrate, tautomer, or optical isomer thereof.
EE15. A compound of formula (II):

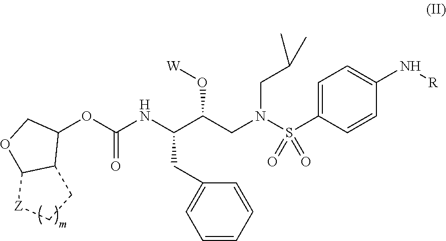

wherein
W is H, deuterium, or PO₃H;
Z is CH₂ or a heteroatom selected from O, S, and N;
m is an integer from 0 to 2; and
R is selected from the group consisting of (a), (b), (c) and (d):

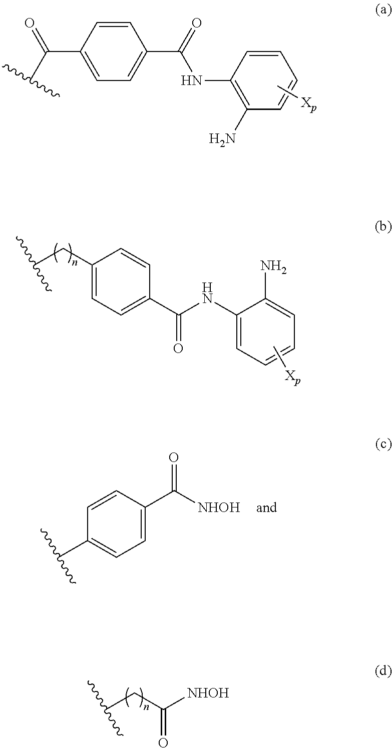

wherein Xₚ is halogen, wherein p is an integer from 1 to 4; and
each n is an integer from 0 to 6;
or a pharmaceutically acceptable salt, hydrate, tautomer, or optical isomer thereof.
EE16. The compound of EE15, wherein X is fluorine.
EE17. A pharmaceutical composition comprising a compound of EE1 or a pharmaceutically acceptable salt, hydrate, tautomer, or optical isomer thereof, and a pharmaceutically acceptable carrier, excipient, or diluent.
EE18. A pharmaceutical composition comprising a compound of formula (II) or a pharmaceutically acceptable salt, hydrate, tautomer, or optical isomer thereof, and a pharmaceutically acceptable carrier, excipient, or diluent.
EE19. A method of dual inhibition of histone deacetylase 3 (HDAC3) and human immunodeficiency virus type-1 (HIV-1) protease (PR) in a patient in need thereof, which method comprises administering to the patient an inhibitory amount of the compound of EE1, or a pharmaceutical composition comprising the same and a pharmaceutically acceptable carrier, excipient, or diluent, whereupon HDAC3 and HIV-1PR in the patient are inhibited.
EE20. The method of EE19, wherein the patient has HIV-1 infection.
EE21. The method of EE19, wherein HIV-1 infection is inhibited.
EE22. The method of EE19, wherein HIV-1 infection is eradicated.
EE23. A method of inhibiting cell-to-cell transmission of HIV-1 in a patient in need thereof, which method comprises administering to the patient an inhibitory amount of a compound of EE 1, which inhibits cell-to-cell transmission of HIV-1, or a pharmaceutical composition comprising the same and a pharmaceutically acceptable carrier, excipient, or diluent, whereupon cell-to-cell transmission of HIV-1 in the patient is inhibited.
EE24. The method of EE23, wherein the compound of claim 1 is a compound wherein
R₂ is

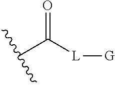

wherein L is C₁-C₅ alkyl, C₆-C₁₂ aryl, or C₅-C₁₂ heteroaryl; and
G is

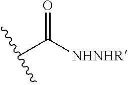

wherein R′ is C₁-C₅ alkyl.
EE25. The method of EE24, wherein the compound of claim 1 is selected from

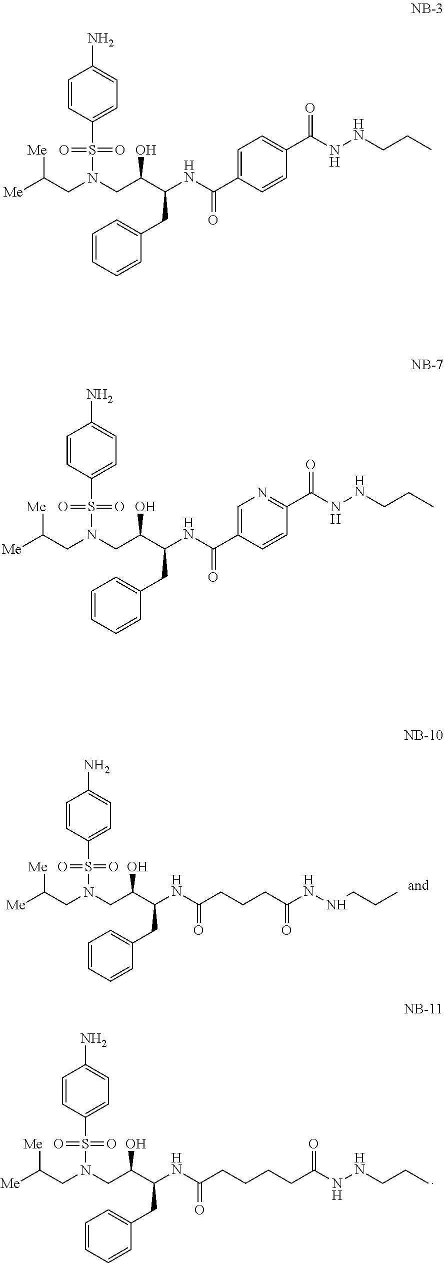 [SMILES: CCCNNC(=O)c1ccc(C(=O)N[C@@H](Cc2ccccc2)[C@H](O)CN(CC(C)C)S(=O)(=O)c2ccc(N)cc2)cc1 ; CCCNNC(=O)c1ccc(C(=O)N[C@@H](Cc2ccccc2)[C@H](O)CN(CC(C)C)S(=O)(=O)c2ccc(N)cc2)cn1 ; CCCNNC(=O)CCCC(=O)N[C@@H](Cc1ccccc1)[C@H](O)CN(CC(C)C)S(=O)(=O)c1ccc(N)cc1 ; CCCNNC(=O)CCCCC(=O)N[C@@H](Cc1ccccc1)[C@H](O)CN(CC(C)C)S(=O)(=O)c1ccc(N)cc1]

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. The terms “including” and “having” are defined as comprising (i.e., open language).

It will be appreciated by persons skilled in the art that the present disclosure is not limited by what has been particularly shown and described herein above. Rather the scope of the present disclosure includes both combinations and sub-combinations of the various features described hereinabove as well as variations and modifications which would occur to persons skilled in the art upon reading the specification and which are not in the prior art.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

We claim:

1. A compound of formula (I): 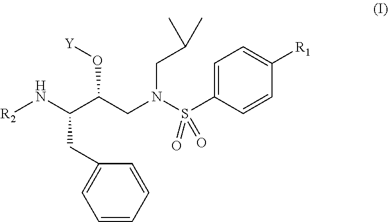
wherein Y is H, deuterium, or PO₃H;
R₁ is NH₂ or a group selected from: 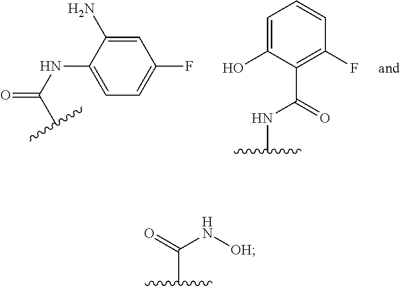 [SMILES: *C(=O)Nc1ccc(F)cc1N ; *NC(=O)c1c(O)cccc1F ; "and" ; *C(=O)NO]
and R₂ is 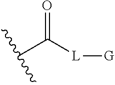
wherein L is O, C₁-C₅ alkyl, C₆-C₁₂ aryl, or C₅-C₁₂ heteroaryl; and
G is selected from the group consisting of (i), (ii), (iii), (iv), and (v): 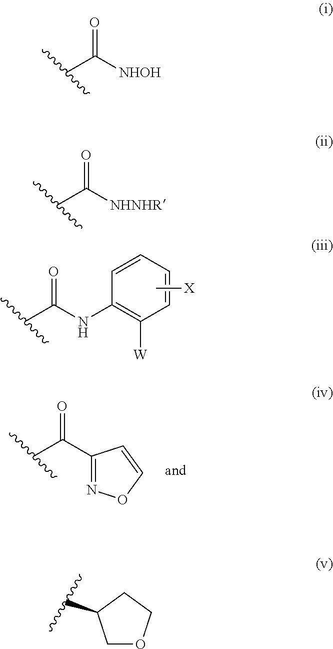
wherein R′ is C₁-C₅ alkyl;
X is H or halogen; and
W is OH, NH₂ or SCH₃;
with the proviso that when G is (v), R₁ is not NH₂,
or a pharmaceutically acceptable salt, hydrate, tautomer, or optical isomer thereof.

2. The compound of claim 1, wherein R₁ is the group selected from:

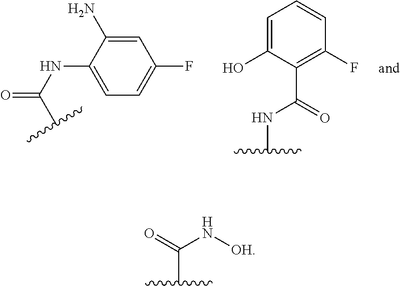 [SMILES: *C(=O)Nc1ccc(F)cc1N ; *NC(=O)c1c(O)cccc1F ; "and" ; *C(=O)NO]

3. The compound of claim 1, wherein R₁ is NH₂;
and R₂ is 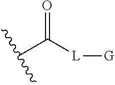
wherein L is o, C₁-C₅ alkyl, C₆-C₁₂ aryl, or C₅-C₁₂ heteroaryl; and
G is selected from the group consisting of (i), (ii), (iii), and (iv): 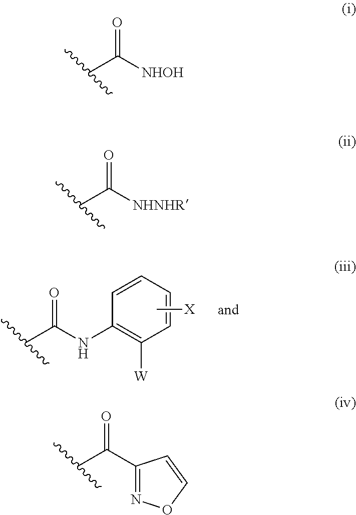
wherein R′ is C₁-C₅ alkyl;
X is H or halogen; and
W is OH, NH₂ or SCH₃.

4. The compound of claim 1, wherein R₂ is 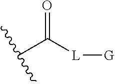
wherein L is O; and
G is 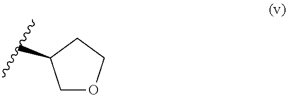 [SMILES: *[C@H]1CCOC1]
with the proviso that when G is (v), R₁ is not NH₂.

5. The compound of claim 4, wherein R₁ is selected from:

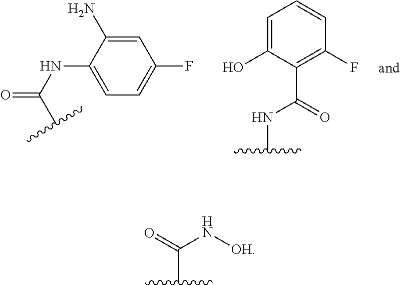 [SMILES: *C(=O)Nc1ccc(F)cc1N ; *NC(=O)c1c(O)cccc1F ; "and" ; *C(=O)NO]

6. The compound of claim 1, wherein R₂ is 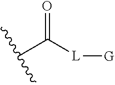
wherein L is C₆-C₁₂ aryl or C₅-C₁₂ heteroaryl; and G is selected from the group consisting of (i), (ii), and (iii): 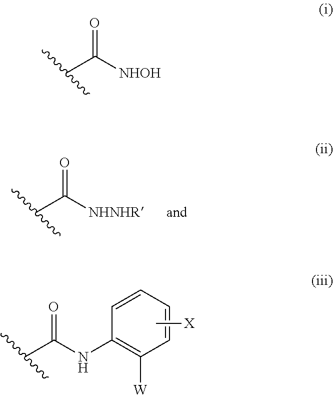
wherein R′ is C₁-C₅ alkyl;
X is H or halogen; and
W is NH₂.

7. The compound of claim 6, wherein L is phenyl or pyridyl.

8. The compound of claim 1, wherein the compound of formula (I) is: 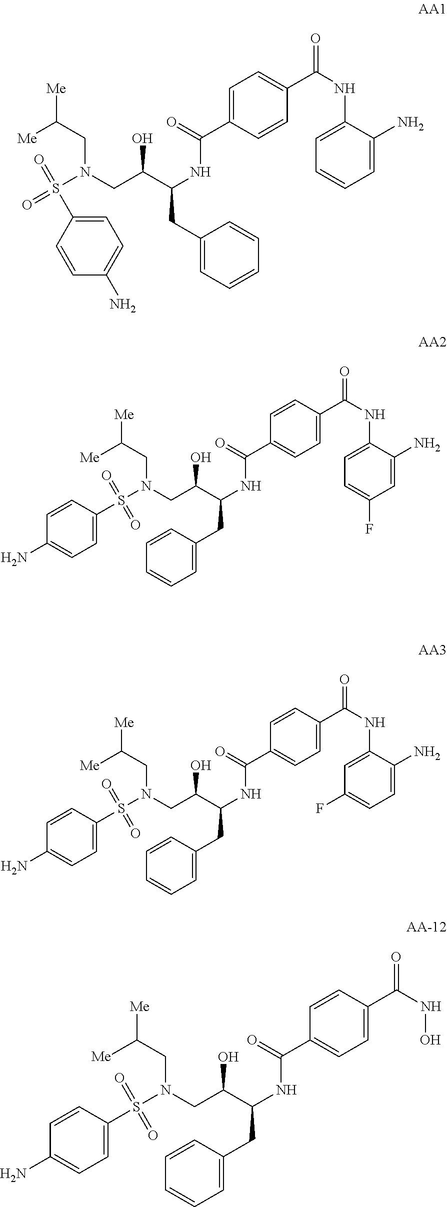 [SMILES: CC(C)CN(C[C@@H](O)[C@H](Cc1ccccc1)NC(=O)c1ccc(C(=O)Nc2ccccc2N)cc1)S(=O)(=O)c1ccc(N)cc1 ; CC(C)CN(C[C@@H](O)[C@H](Cc1ccccc1)NC(=O)c1ccc(C(=O)Nc2ccc(F)cc2N)cc1)S(=O)(=O)c1ccc(N)cc1 ; CC(C)CN(C[C@@H](O)[C@H](Cc1ccccc1)NC(=O)c1ccc(C(=O)Nc2cc(F)ccc2N)cc1)S(=O)(=O)c1ccc(N)cc1 ; CC(C)CN(C[C@@H](O)[C@H](Cc1ccccc1)NC(=O)c1ccc(C(=O)NO)cc1)S(=O)(=O)c1ccc(N)cc1] 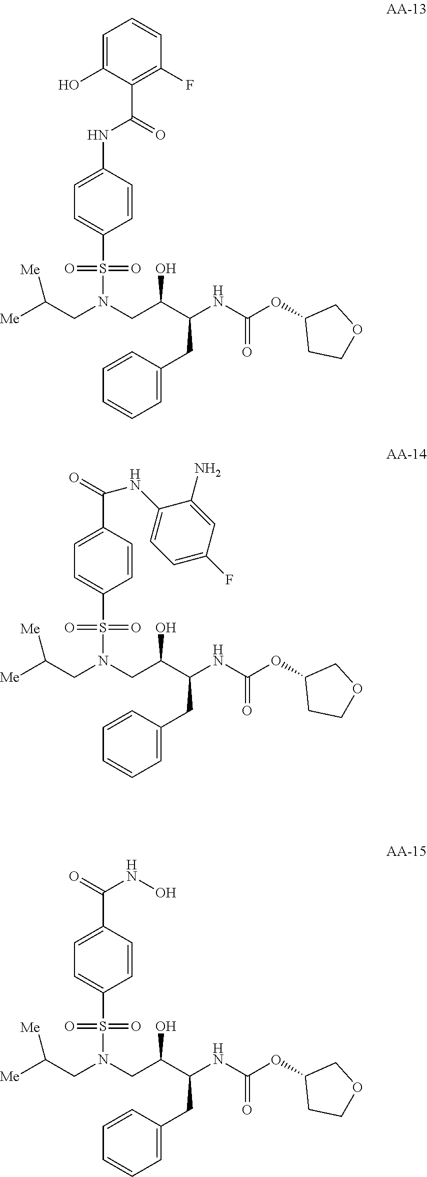 [SMILES: CC(C)CN(C[C@@H](O)[C@H](Cc1ccccc1)NC(=O)O[C@H]1CCOC1)S(=O)(=O)c1ccc(NC(=O)c2c(O)cccc2F)cc1 ; CC(C)CN(C[C@@H](O)[C@H](Cc1ccccc1)NC(=O)O[C@H]1CCOC1)S(=O)(=O)c1ccc(C(=O)Nc2ccc(F)cc2N)cc1 ; CC(C)CN(C[C@@H](O)[C@H](Cc1ccccc1)NC(=O)O[C@H]1CCOC1)S(=O)(=O)c1ccc(C(=O)NO)cc1] 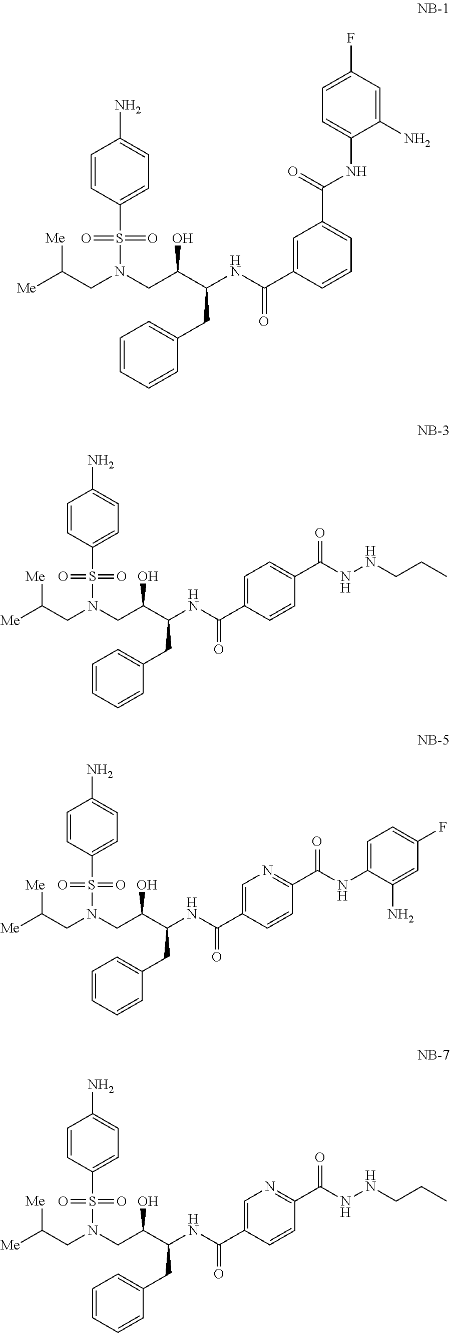 [SMILES: CC(C)CN(C[C@@H](O)[C@H](Cc1ccccc1)NC(=O)c1cccc(C(=O)Nc2ccc(F)cc2N)c1)S(=O)(=O)c1ccc(N)cc1 ; CCCNNC(=O)c1ccc(C(=O)N[C@@H](Cc2ccccc2)[C@H](O)CN(CC(C)C)S(=O)(=O)c2ccc(N)cc2)cc1 ; CC(C)CN(C[C@@H](O)[C@H](Cc1ccccc1)NC(=O)c1ccc(C(=O)Nc2ccc(F)cc2N)nc1)S(=O)(=O)c1ccc(N)cc1 ; CCCNNC(=O)c1ccc(C(=O)N[C@@H](Cc2ccccc2)[C@H](O)CN(CC(C)C)S(=O)(=O)c2ccc(N)cc2)cn1] 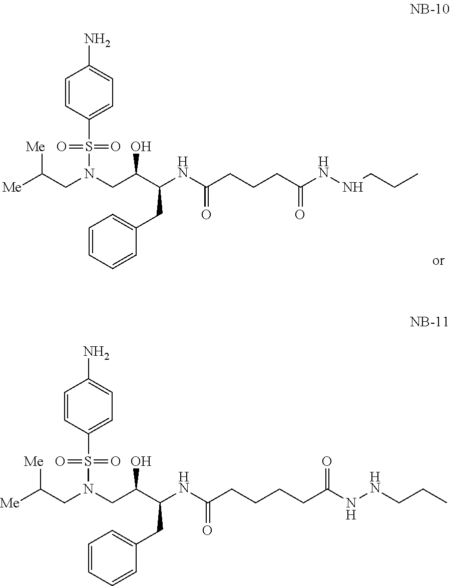 [SMILES: CCCNNC(=O)CCCC(=O)N[C@@H](Cc1ccccc1)[C@H](O)CN(CC(C)C)S(=O)(=O)c1ccc(N)cc1 ; CCCNNC(=O)CCCCC(=O)N[C@@H](Cc1ccccc1)[C@H](O)CN(CC(C)C)S(=O)(=O)c1ccc(N)cc1]
or a pharmaceutically acceptable salt, hydrate, tautomer, or optical isomer thereof.

9. The compound of claim 1, wherein the compound of formula (I) is a compound of formula (IA): 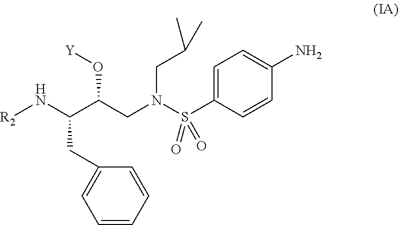
wherein
Y is H, deuterium or PO₃H; and
R is selected from the group consisting of (a), (b), (c) and (d): 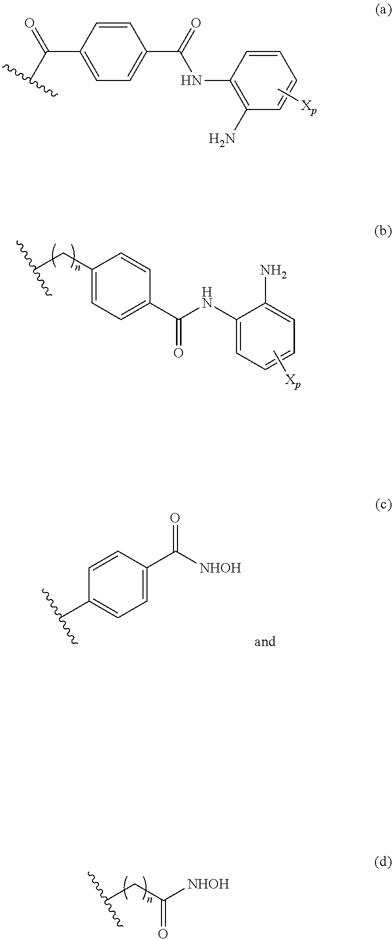
wherein Xₚ is halogen, wherein p is an integer from 1 to 4; and
n is an integer from 0 to 6;
or a pharmaceutically acceptable salt, hydrate, tautomer, or optical isomer thereof.

10. The compound of claim 9, wherein X is fluorine.

11. The compound of claim 9, wherein R is: 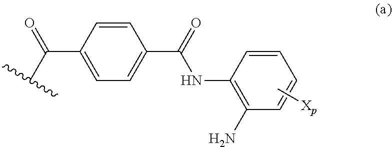
wherein Xₚ is as defined above.

12. The compound of claim 9, wherein R is: 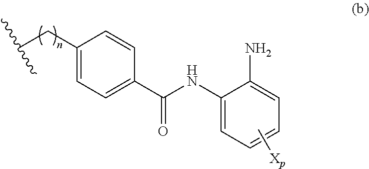
wherein Xₚ and n are as defined above.

13. The compound of claim 9, wherein R is:

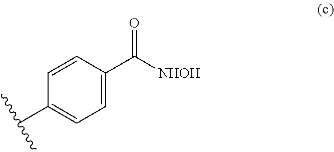

14. The compound of claim 9, wherein the compound of formula (IA) is: 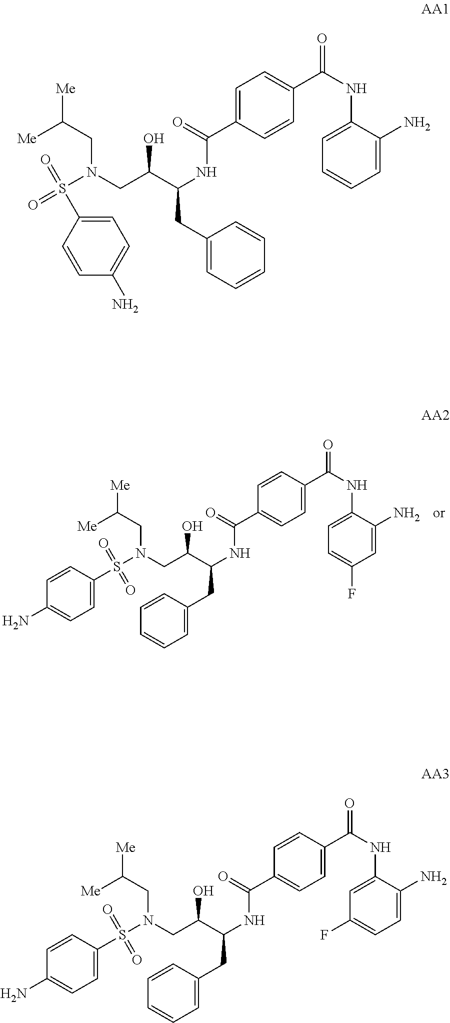 [SMILES: CC(C)CN(C[C@@H](O)[C@H](Cc1ccccc1)NC(=O)c1ccc(C(=O)Nc2ccccc2N)cc1)S(=O)(=O)c1ccc(N)cc1 ; CC(C)CN(C[C@@H](O)[C@H](Cc1ccccc1)NC(=O)c1ccc(C(=O)Nc2ccc(F)cc2N)cc1)S(=O)(=O)c1ccc(N)cc1 ; CC(C)CN(C[C@@H](O)[C@H](Cc1ccccc1)NC(=O)c1ccc(C(=O)Nc2cc(F)ccc2N)cc1)S(=O)(=O)c1ccc(N)cc1]
or a pharmaceutically acceptable salt, hydrate, tautomer, or optical isomer thereof.

15. A compound of formula (II): 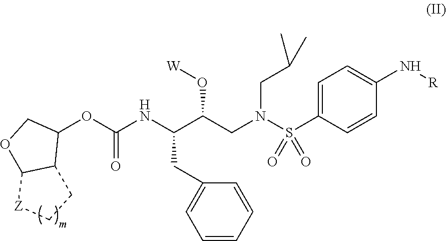
wherein
W is H, deuterium or PO₃H;
Z is CH₂ or a heteroatom selected from O, S, and N;
m is an integer from 0 to 2; and
R is selected from the group consisting of (a), (b), (c) and (d): 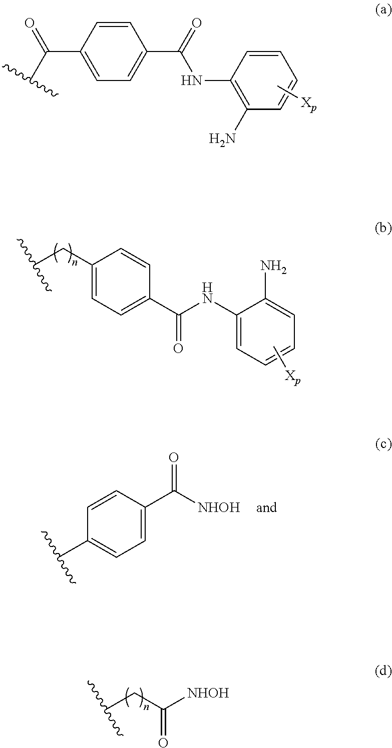
wherein Xₚ is halogen, wherein p is an integer from 1 to 4; and
each n is an integer from 0 to 6;
or a pharmaceutically acceptable salt, hydrate, tautomer, or optical isomer thereof.

16. The compound of claim 15, wherein X is fluorine.

17. A pharmaceutical composition comprising a compound of claim 1 or a pharmaceutically acceptable salt, hydrate, tautomer, or optical isomer thereof, and a pharmaceutically acceptable carrier, excipient, or diluent.

18. A pharmaceutical composition comprising a compound of formula (II) or a pharmaceutically acceptable salt, hydrate, tautomer, or optical isomer thereof, and a pharmaceutically acceptable carrier, excipient, or diluent.

19. A method of dual inhibition of histone deacetylase 3 (HDAC3) and human immunodeficiency virus type −1 (HIV-1) protease (PR) in a patient in need thereof, which method comprises administering to the patient an inhibitory amount of the compound of claim 1, or a pharmaceutical composition comprising the same and a pharmaceutically acceptable carrier, excipient, or diluent, whereupon HDAC3 and HIV-1PR in the patient are inhibited.

20. The method of claim 19, wherein the patient has HIV-1 infection.

21. The method of claim 19, wherein HIV-1 infection is inhibited.

22. The method of claim 19, wherein HIV-1 infection is eradicated.

23. A method of inhibiting cell-to-cell transmission of HIV-1 in a patient in need thereof, which method comprises administering to the patient an inhibitory amount of a compound of claim 1, which inhibits cell-to-cell transmission of HIV-1, or a pharmaceutical composition comprising the same and a pharmaceutically acceptable carrier, excipient, or diluent, whereupon cell-to-cell transmission of HIV-1 in the patient is inhibited.

24. The method of claim 23, wherein the compound of claim 1 is a compound wherein
R₂ is 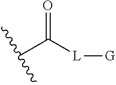
wherein L is C₁-C₅ alkyl, C₆-C₁₂ aryl, or C₅-C₁₂ heteroaryl; and
G is 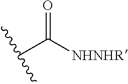
wherein R′ is C₁-C₅ alkyl.

25. The method of claim 24, wherein the compound of claim 1 is selected from 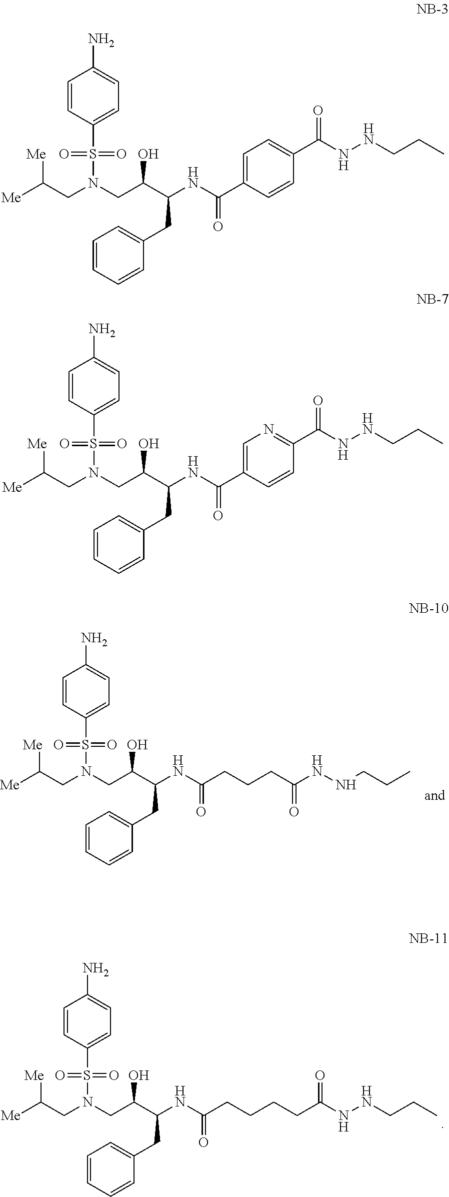 [SMILES: CCCNNC(=O)c1ccc(C(=O)N[C@@H](Cc2ccccc2)[C@H](O)CN(CC(C)C)S(=O)(=O)c2ccc(N)cc2)cc1 ; CCCNNC(=O)c1ccc(C(=O)N[C@@H](Cc2ccccc2)[C@H](O)CN(CC(C)C)S(=O)(=O)c2ccc(N)cc2)cn1 ; CCCNNC(=O)CCCC(=O)N[C@@H](Cc1ccccc1)[C@H](O)CN(CC(C)C)S(=O)(=O)c1ccc(N)cc1 ; CCCNNC(=O)CCCCC(=O)N[C@@H](Cc1ccccc1)[C@H](O)CN(CC(C)C)S(=O)(=O)c1ccc(N)cc1]
